(19) United States
(12) Patent Application Publication
(10) Pub. No.: US 2023/0313214 A1
(43) Pub. Date: Oct. 5, 2023

(54) PROMOTER ELEMENTS FOR IMPROVED POLYNUCLEOTIDE EXPRESSION IN PLANTS

(71) Applicant: BENSON HILL, INC., St. Louis, MO (US)

(72) Inventor: Ross A. Johnson, Olivette, MO (US)

(73) Assignee: Benson Hill, Inc., St. Louis, MO (US)

(21) Appl. No.: 18/173,947

(22) Filed: Feb. 24, 2023

Related U.S. Application Data
(60) Provisional application No. 63/314,082, filed on Feb. 25, 2022.

(51) Int. Cl. C12N 15/82; C12N 9/22; C07K 14/415

(52) U.S. Cl. CPC C12N 15/8216; C12N 15/8213; C12N 9/22; C07K 14/415; C12N 2310/20

(57) ABSTRACT

Provided herein are compositions and methods for expressing a polynucleotide of interest in a plant or plant part. Compositions include nucleic acid molecules comprising a promoter sequence, and DNA constructs comprising the promoter molecule operably linked to one or more polynucleotides of interest. Plants and plant parts comprising the compositions or regenerated according to the methods are also provided.

### CROSS REFERENCE TO RELATED APPLICATIONS

This application claims priority to U.S. Provisional Application No. 63/314,082 filed on Feb. 25, 2022, the content of which is incorporated herein by reference in its entirety.

### FIELD OF THE INVENTION

The present disclosure relates to compositions and methods for expressing a polynucleotide of interest in a plant or plant part.

### SEQUENCE LISTING

This application contains a Sequence Listing which is submitted herewith in electronically readable format. The Sequence Listing file was created on Feb. 24, 2023, is named “B88552_1490_SL.xml” and its size is 181 kb. The entire contents of the Sequence Listing file are incorporated by reference herein.

### BACKGROUND OF THE INVENTION

Expressing a polynucleotide of interest in a plant or plant part is useful for product development. For instance, strong expression of editing reagents, e.g., a nuclease and a guide RNA (gRNA), across proliferating cell types in a transformed plant can enable an effective incidence of plants with desirable edits in a target sequence that are transmitted to the secondary-transformants and improve efficiency of introducing heritable edits (editing %). Additionally, the expression of selectable markers in a ubiquitous constitutive pattern can enable favorable incidence of primary-transformants regenerated relative to the number of transformed explants and improve transformation efficiency (transformation %). A favorable interplay between selectable marker and editing reagent expression can enable transgene-free segregants to be recovered from the secondary-transformant generation. Put together, effective expression elements are needed to achieve favorable performance metrics in commercial editing applications.

### SUMMARY OF THE INVENTION

Compositions and methods for regulating gene expression in a plant or plant part are provided. Compositions can include nucleic acid molecules comprising a promoter molecule for expressing a polynucleotide of interest, or DNA constructs comprising the promoters operably linked to polynucleotides of interest. The promoters can enable high expression levels and/or favorable expression patterns of one or more polynucleotides of interest, including editing reagents, e.g., nucleases and guide RNAs. Methods of expressing a polynucleotide of interest in a plant or plant part, methods of transforming a plant or plant part, and plants and plant parts comprising the compositions or being regenerated according to the methods of the present disclosure are also described.

In one aspect, the present disclosure provides a nucleic acid molecule comprising a promoter sequence, wherein the promoter sequence comprises one or more deletions, substitutions, and/or insertions relative to the nucleic acid sequence of any one of SEQ ID NOs: 1-4, 9-39, and 43-46; shares at least 75% sequence identity with any one of SEQ ID NOs: 1-4 and 9-39, and 43-46; and retains transcription initiation function. In some embodiments, said one or more deletions, substitutions, and/or insertions are located in a 3′ region of said promoter sequence. In some embodiments, the promoter sequence shares at least 80% sequence identity with any one of SEQ ID NOs: 5-8, or comprises the nucleic acid sequence of any one of SEQ ID NOs: 5-8, and wherein the promoter sequence retains transcription initiation function. In some embodiments, the nucleic acid molecule further comprises a 5′ untranslated region (UTR) sequence, a 5′UTR intron sequence, an exon sequence from a coding region, and/or an intron sequence from a coding region of a plant genome.

In one aspect, the present disclosure provides a DNA construct comprising, in operable linkage: (a) a nucleic acid molecule comprising a promoter sequence, wherein the promoter sequence comprises one or more deletions, substitutions, and/or insertions relative to the nucleic acid sequence of any one of SEQ ID NOs: 1-4, 9-39, and 43-46, shares at least 75% sequence identity with any one of SEQ ID NOs: 1-4, 9-39, and 43-46, and retains transcription initiation function; a promoter molecule comprising a nucleic acid sequence that shares at least 80% sequence identity with any one of SEQ ID NOs: 1-4, 9-39, and 43-46, and retains transcription initiation function; or a promoter molecule comprising a nucleic acid sequence of any one of SEQ ID NOs: 1-4, 9-39, and 43-46; and (b) a polynucleotide of interest. In specific embodiments, the DNA construct comprises, in operable linkage: (a) a promoter molecule comprising a nucleic acid sequence that shares at least 80% sequence identity with any one of SEQ ID NOs: 1-4, and retains transcription initiation function, or a promoter molecule comprising a nucleic acid sequence of any one of SEQ ID NOs: 1-4; and (b) a polynucleotide of interest. In some embodiments, the promoter molecule further comprises a 5′ untranslated region (UTR) sequence, a 5′UTR intron sequence, an exon sequence from a coding region, and/or an intron sequence from a coding region of a plant genome.

In some embodiments, the polynucleotide of interest encodes a guide RNA and/or a nuclease. In some embodiments, the polynucleotide of interest encodes a guide RNA, and wherein the DNA construct further comprises, in operable linkage: (a) a nucleic acid molecule comprising a promoter sequence, wherein the promoter sequence comprises one or more deletions, substitutions, and/or insertions relative to the nucleic acid sequence of any one of SEQ ID NOs: 1-4, 9-39, and 43-46, shares at least 75% sequence identity with any one of SEQ ID NOs: 1-4, 9-39, and 43-46, and retains transcription initiation function; a promoter molecule comprising a nucleic acid sequence that has at least 80% sequence identity to any one of SEQ ID NOs: 1-4, 9-39, 43-52, and 56-61, and retains transcription initiation function; or a promoter molecule comprising a nucleic acid sequence of any one of SEQ ID NOs: 1-4, 9-39, 43-52, and 56-61; and (b) a polynucleotide of interest encoding a nuclease. In some embodiments, the polynucleotide of interest encodes a nuclease, and wherein the DNA construct further comprises, in operable linkage: (a) a nucleic acid molecule comprising a promoter sequence, wherein the promoter sequence comprises one or more deletions, substitutions, and/or insertions relative to the nucleic acid sequence of any one of SEQ ID NOs: 1-4, 9-39, and 43-46, shares at least 75% sequence identity with any one of SEQ ID NOs: 1-4, 9-39, and 43-46, and retains transcription initiation function; a promoter molecule comprising a nucleic acid sequence that has at least 80% sequence identity to any one of SEQ ID NOs: 1-4, 47-49, 51, and 53, and retains transcription initiation function; or a promoter molecule comprising a nucleic acid sequence of any one of SEQ ID NOs: 1-4, 47-49, 51, and 53; and (b) a polynucleotide of interest encoding a guide RNA. In some embodiments, the nuclease is a clustered, regularly interspaced, short palindromic repeats (CRISPR)-associated Cas endonuclease. In some embodiments, the CRISPR nuclease is a Cas12a nuclease. In some embodiments, the Cas12a nuclease is a McCpf1 nuclease. In some embodiments, the nuclease is further operably linked to one or more nuclear localization sequences (NLSs) (e.g., SV40-nucleophosmin 2) and/or one or more epitope tags.

In some embodiments, the DNA construct comprises a nucleic acid molecule encoding a selectable marker, morphogen gene, reporter gene, and/or a regulatory RNA, operably linked to a promoter molecule. In some embodiments, the promoter molecule operably linked to the regulatory RNA comprises: a nucleic acid molecule comprising a promoter sequence, wherein the promoter sequence comprises one or more deletions, substitutions, and/or insertions relative to the nucleic acid sequence of any one of SEQ ID NOs: 1-4, 9-39, and 43-46, shares at least 75% sequence identity with any one of SEQ ID NOs: 1-4, 9-39, and 43-46, and retains transcription initiation function; a nucleic acid sequence that has at least 80% sequence identity to any one of SEQ ID NOs: 1-4, 47-49, 51, and 53, and retains transcription initiation function; or a nucleic acid sequence of any one of SEQ ID NOs: 1-4, 47-49, 51, and 53.

In one aspect, the present disclosure provides a cell comprising the nucleic acid molecule provided herein or the DNA construct provided herein. In some embodiments, the cell is selected from the group consisting of a plant cell, a bacterial cell, and a fungal cell.

In one aspect, the present disclosure provides a plant or plant part comprising the nucleic acid molecule comprising a promoter sequence provided herein, the DNA construct provided herein, or the cell provided herein. In a particular aspect, the present disclosure provides a plant or plant part comprising, in operable linkage: (a) a nucleic acid molecule comprising a promoter sequence, wherein the promoter sequence comprises one or more deletions, substitutions, and/or insertions relative to the nucleic acid sequence of any one of SEQ ID NOs: 1-4, 9-39, and 43-46, shares at least 75% sequence identity with any one of SEQ ID NOs: 1-4, 9-39, and 43-46; and retains transcription initiation function; a promoter molecule comprising a nucleic acid sequence that has at least 80% sequence identity to any one of SEQ ID NOs: 1-4, 9-39, and 43-46, and retains transcription initiation function; or a promoter molecule comprising a nucleic acid sequence of any one of SEQ ID NOs: 1-4, 9-39, and 43-46; and (b) a polynucleotide of interest. In some embodiments, said one or more deletions, substitutions, and/or insertions are located in a 3′ region of said promoter sequence. In some embodiments, the promoter sequence of the plant or plant part shares at least 80% sequence identity with any one of SEQ ID NOs: 5-8, or comprises the nucleic acid sequence of any one of SEQ ID NOs: 5-8, and wherein the promoter sequence retains transcription initiation function. In some aspects, the promoter sequence or the promoter molecule of the plant or plant part comprises a 5′ untranslated region (UTR) sequence, a 5′UTR intron sequence, an exon sequence from a coding region, and/or an intron sequence from a coding region of a plant genome. In some embodiments, the nucleic acid molecule, the DNA construct, or part thereof is stably inserted in the genome of said plant or plant part.

In some embodiments, the plant or plant part is selected from the group consisting of pea (Pisum sativum), soybean (Glycine max), peanut (Arachis hypogaea), chickpea (Cicer arietinum), white lupin (Lupinus albus), birdsfood trefoil (Lotus japonicus), barrel medic (Medicago truncatula), beans (Phaseolus spp.), common bean (Phaseolus vulgaris), clover (Trifohum spp.), red clover (Trifohum pratense), cowpea (Vigna unguiculata), tomato (Solanum lycopersicum), fava bean (Viciafaba), mung bean (Vigna radiata), lentils (Lens culinaris, Lens esculenta), lupins (Lupinus spp.), mesquite (Prosopis spp.), carob (Ceratonia siliqua), tamarind (Tamarindus indica), alfalfa (Medicago sativa), licorice (Glycyrrhiza glabra), Brassica species, Brassica napus, Brassica rapa, Brassica juncea, alfalfa (Medicago sativa), rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), corn (Zea mays), sunflower (Helianthus annuus), safflower (Carthamus tinctorius), wheat (Triticum aestivum), tobacco (Nicotiana tabacum), potato (Solanum tuberosum), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers. In a specific embodiment, the plant or plant part is a Pisum sativum plant or a Pisum sativum plant part.

In one aspect, the present disclosure provides a method of expressing a polynucleotide of interest in a plant or plant part comprising introducing a DNA construct into the plant or plant part, wherein the DNA construct comprises, in operable linkage: (a) a nucleic acid molecule comprising a promoter sequence, wherein the promoter sequence comprises one or more deletions, substitutions, and/or insertions relative to the nucleic acid sequence of any one of SEQ ID NOs: 1-4, 9-39, and 43-46, shares at least 75% sequence identity with any one of SEQ ID NOs: 1-4, 9-39, and 43-46, and retains transcription initiation function; a promoter molecule comprising a nucleic acid sequence that has at least 80% sequence identity to any one of SEQ ID NOs: 1-4, 9-39, and 43-46, and retains transcription initiation function; or a promoter molecule comprising a nucleic acid sequence of any one of SEQ ID NOs: 1-4, 9-39, and 43-46; and (b) a polynucleotide of interest.

In one aspect, the present disclosure provides a method of transforming a plant or plant part, comprising: introducing a DNA construct into a plant cell, wherein the DNA construct comprises, in operable linkage: (a) a nucleic acid molecule comprising a promoter sequence, wherein the promoter sequence comprises one or more deletions, substitutions, and/or insertions relative to the nucleic acid sequence of any one of SEQ ID NOs: 1-4, 9-39, and 43-46, shares at least 75% sequence identity with any one of SEQ ID NOs: 1-4, 9-39, and 43-46, and retains transcription initiation function; a promoter molecule comprising a nucleic acid sequence that shares at least 80% sequence identity with any one of SEQ ID NOs: 1-4, 9-39, and 43-46, and retains transcription initiation function; or a promoter molecule comprising a nucleic acid sequence of any one of SEQ ID NOs: 1-4, 9-39, and 43-46; and (b) a polynucleotide of interest; and regenerating a transformed plant or plant part from said plant cell.

In some embodiments, said one or more deletions, substitutions, and/or insertions of the promoter sequence are located in a 3′ region of said promoter sequence. In some embodiments, the promoter sequence shares at least 80% sequence identity with any one of SEQ ID NOs: 5-8, or comprises the nucleic acid sequence of any one of SEQ ID NOs: 5-8, and wherein the promoter sequence retains transcription initiation function. In specific embodiments of the methods of the present disclosure, the DNA construct comprises, in operable linkage: (a) a promoter molecule comprising a nucleic acid sequence that shares at least 80% sequence identity with any one of SEQ ID NOs: 1-4, and retains transcription initiation function; or a promoter molecule comprising a nucleic acid sequence of any one of SEQ ID NOs: 1-4; and (b) a polynucleotide of interest. In some embodiments, the promoter sequence or the promoter molecule further comprises a 5′ untranslated region (UTR) sequence, a 5′UTR intron sequence, an exon sequence from a coding region, and/or an intron sequence from a coding region of a plant genome.

In some embodiments, the polynucleotide of interest encodes a guide RNA and/or a nuclease. In some embodiments, the polynucleotide of interest encodes a guide RNA, and wherein the DNA construct further comprises, in operable linkage: (a) a nucleic acid molecule comprising a promoter sequence, wherein the promoter sequence comprises one or more deletions, substitutions, and/or insertions relative to the nucleic acid sequence of any one of SEQ ID NOs: 1-4, 9-39, and 43-46, shares at least 75% sequence identity with any one of SEQ ID NOs: 1-4, 9-39, and 43-46, and retains transcription initiation function; a promoter molecule comprising a nucleic acid sequence that has at least 80% sequence identity to any one of SEQ ID NOs: 1-4, 9-39, 43-52, and 56-61, and retains transcription initiation function; or a promoter molecule comprising a nucleic acid sequence of any one of SEQ ID NOs: 1-4, 9-39, 43-52, and 56-61; and (b) a polynucleotide of interest encoding a nuclease. In some embodiments, the polynucleotide of interest encodes a nuclease, and wherein the DNA construct further comprises, in operable linkage: (a) a nucleic acid molecule comprising a promoter sequence, wherein the promoter sequence comprises one or more deletions, substitutions, and/or insertions relative to the nucleic acid sequence of any one of SEQ ID NOs: 1-4, 9-39, and 43-46, shares at least 75% sequence identity with any one of SEQ ID NOs: 1-4, 9-39, and 43-46, and retains transcription initiation function; a promoter molecule comprising a nucleic acid sequence that has at least 80% sequence identity to any one of SEQ ID NOs: 1-4, 47-49, 51, and 53, and retains transcription initiation function; or a promoter molecule comprising a nucleic acid sequence of any one of SEQ ID NOs: 1-4, 47-49, 51, and 53; and (b) a polynucleotide of interest encoding a guide RNA. In some embodiments, the nuclease is a clustered, regularly interspaced, short palindromic repeats (CRISPR)-associated Cas endonuclease. In some embodiments, the CRISPR nuclease is a Cas12a nuclease. In some embodiments, the Cas12a nuclease is a McCpf1 nuclease. In some embodiments, the nuclease is further operably linked to one or more nuclear localization sequences (NLSs, e.g., SV40-nucleophosmin 2) and/or one or more epitope tags.

In some embodiments, the DNA construct according to the methods of the present disclosure comprises a nucleic acid molecule encoding a selectable marker and/or a regulatory RNA, operably linked to a promoter molecule. In some embodiments, the promoter molecule operably linked to the regulatory RNA comprises: a nucleic acid molecule comprising a promoter sequence, wherein the promoter sequence comprises one or more deletions, substitutions, and/or insertions relative to the nucleic acid sequence of any one of SEQ ID NOs: 1-4, 9-39, and 43-46, shares at least 75% sequence identity with any one of SEQ ID NOs: 1-4, 9-39, and 43-46, and retains transcription initiation function; a nucleic acid sequence that has at least 80% sequence identity to any one of SEQ ID NOs: 1-4, 47-49, 51, and 53, and retains transcription initiation function; or a nucleic acid sequence of any one of SEQ ID NOs: 1-4, 47-49, 51, and 53.

In some embodiments, the nucleic acid molecule, the DNA construct, or part thereof is stably inserted in the genome of said plant or plant part according to the methods of the present disclosure.

In some embodiments, expression or function of one or more molecules encoded by the polynucleotide(s) of interest is increased in the plant or plant part relative to a control plant or plant part comprising the polynucleotide(s) of interest operably linked to a control promoter. In some embodiments, the control promoter does not comprise a promoter sequence (a) comprising one or more deletions, substitutions, and/or insertions relative to the nucleic acid sequence of any one of SEQ ID NOs: 1-4, 9-39, and 43-46, shares at least 75% sequence identity with any one of SEQ ID NOs: 1-4, 9-39, and 43-46, and retains transcription initiation function, (b) comprising a nucleic acid sequence that has at least 80% sequence identity to any one of SEQ ID NOs: 1-4, 9-39, 43-52, and 56-61, and retains transcription initiation function; or (c) comprising a nucleic acid sequence of any one of SEQ ID NOs: 1-4, 9-39, 43-52, and 56-61.

In some embodiments, one or more molecules encoded by the polynucleotide(s) of interest are a guide RNA and/or a nuclease, and their expression or function is increased in the plant or plant part relative to a control plant or plant part comprising the guide RNA and/or nuclease operably linked to a control promoter. In further embodiments, an efficiency of introducing a mutation to a genome of the plant or plant part is increased according to the methods of the present disclosure relative to the control plant or plant part. In some embodiments, an efficiency of introducing a heritable mutation to a genome of a stably transformed and regenerated plant or plant part consistently across plant tissue is increased according to the methods of the present disclosure relative to the control plant or plant part.

In some embodiments of the methods provided herein, said plant or plant part is selected from the group consisting of pea (Pisum sativum), soybean (Glycine max), peanut (Arachis hypogaea), chickpea (Cicer arietinum), white lupin (Lupinus albus), birdsfood trefoil (Lotus japonicus), barrel medic (Medicago truncatula), beans (Phaseolus spp.), common bean (Phaseolus vulgaris), clover (Trifolium spp.), red clover (Trifolium pratense), cowpea (Vigna unguiculata), tomato (Solanum lycopersicum), fava bean (Viciafaba), mung bean (Vigna radiata), lentils (Lens culinaris, Lens esculenta), lupins (Lupinus spp.), mesquite (Prosopis spp.), carob (Ceratonia siliqua), tamarind (Tamarindus indica), alfalfa (Medicago sativa), licorice (Glycyrrhiza glabra), Brassica species, Brassica napus, Brassica rapa, Brassica juncea, alfalfa (Medicago sativa), rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), corn (Zea mays), sunflower (Helianthus annuus), safflower (Carthamus tinctorius), wheat (Triticum aestivum), tobacco (Nicotiana tabacum), potato (Solanum tuberosum), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers. In specific embodiments, said plant or plant part is a Pisum sativum plant or a Pisum sativum plant part.

In a certain aspect, provided herein is a plant or plant part produced by the methods of the present disclosure, wherein said plant or plant part comprises the nucleic acid molecule, DNA construct, or part thereof of the present disclosure.

### BRIEF DESCRIPTION OF THE DRAWINGS

FIG. 1 depicts editing frequencies in pea protoplasts co-transfected with a guide RNA construct and a nuclease construct each operably linked to a control promoter (AtUBI11p) or a test promoter set forth in the figure. The nucleic acid sequences of the AtUBI11p (control), PvEF1A-4Bp, PsEF 1A-1p, PsEF 1A-6p, PsUBI3p, and PsEF 1A-7p promoters are set forth as SEQ ID NOs: 47, 36, 2, 3, 48, and 4, respectively. Unless specified in the figure, the guide RNA or the nuclease was operably linked to the control promoter.

FIG. 2 depicts editing frequencies in tomato protoplasts co-transfected with a CRISPR-Cas12a (Cpf1) nuclease construct operably linked to a promoter set forth in the figure and a guide RNA construct. The guide RNA was operably linked to the PsUBI3p promoter. The nucleic acid sequences of the AtUBI11p (control), PsUBI3p, SlUBI7p, SlUBI11p, SlEF1A6p, PsEF1A-1p, and PsEF1A-7p, promoters are set forth as SEQ ID NOs: 47, 48, 50, 54, 55, 2, and 4, respectively.

FIG. 3 depicts percentages of soybean TO plants having >25% edits over total T0 plants screened. T0 plants were stably transformed with a guide RNA and a CRISPR-Cas12a (Cpf1) nuclease each operably linked to the promoters described in the figure.

FIG. 4 depicts the incidence of overall profiled editing events in T0 soybean plants, which comprise the incidence of “fixed” edit events (i.e., a consistent insertion-deletion profile across proliferating tissue in a mid-development T0 plant), and the incidence of “unfixed” edits (i.e., an inconsistent insertion-deletion profile across the T0 plant tissue) in the T0 plants stably transformed with a guide RNA and a CRISPR-Cas12a nuclease each operably linked to the promoters described in the figure.

FIG. 5 depicts diagrams of PsEF1A-0p, PsEF1A-1p, PsEF1A-6p, and PsEF1A-7p. Each promoter is shown with a black arrow to scale, with the 5′UTR and 5′UTR intron being annotated in gray as solid and dotted lines, respectively. The nucleic acid sequences of these four promoters are set forth as SEQ ID NOs: 1, 2, 3, and 4, respectively.

### DETAILED DESCRIPTION OF THE INVENTION

The present disclosure now will be described more fully hereinafter. The disclosure may be embodied in many different forms and should not be construed as limited to the aspects set forth herein; rather, these aspects are provided so that this disclosure will satisfy applicable legal requirements.

### I. Definitions

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

As used herein, “a,” “an,” or “the” can mean one or more than one. For example, “a” cell can mean a single cell or a multiplicity of cells. Further, the term “a plant” may include a plurality of plants.

As used herein, unless specifically indicated otherwise, the word “or” is used in the inclusive sense of “and/or” and not the exclusive sense of “either/or.”

The term “about” or “approximately” usually means within 5%, or more preferably within 1%, of a given value or range.

The terms “comprises”, “comprising”, “includes”, “including”, “having” and their conjugates mean “including but not limited to”.

Various embodiments of this disclosure may be presented in a range format. It should be noted that whenever a value or range of values of a parameter are recited, it is intended that values and ranges intermediate to the recited values are also part of this disclosure. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1-10 should be considered to have specifically disclosed subranges such as from 1 to 3, from 1 to 4, from 1 to 5, from 1 to 6, from 1 to 7, from 1 to 8, from 1 to 9, from 2 to 4, from 2 to 6, from 2 to 8, from 2 to 10, from 3 to 6, etc., as well as individual numbers within that range, for example, 1, 2, 3, 4, 5, 6, 7, 8, 9 and 10. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. The recitation of a numerical range for a variable is intended to convey that the present disclosure may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values and if the variable is inherently continuous.

A “plant” refers to a whole plant, any part thereof, or a cell or tissue culture derived from a plant, comprising any of: whole plants, plant components or organs (e.g., leaves, stems, roots, embryos, pollen, ovules, seeds, grains, leaves, flowers, branches, fruit, pulp, juice, kernels, ears, cobs, husks, stalks, root tips, anthers, etc.), plant tissues, plant cell tissue cultures from which plants can be regenerated, plant calli, plant clumps, seeds, plant cells, protoplasts and/or progeny of the same. A plant cell is a biological cell of a plant, taken from a plant or derived through culture of a cell taken from a plant. Grain is intended to mean the mature seed produced by commercial growers for purposes other than growing or reproducing the species. Progeny, variants, and mutants of the regenerated plants comprising the introduced polynucleotides are also within the scope of the invention. Further provided is a processed plant product (e.g., extract) or byproduct that retains one or more polynucleotides disclosed herein.

As used herein, a “subject plant or plant cell” is one in which genetic alteration, such as a mutation, has been effected as to a polynucleotide of interest, or is a plant or plant cell which is descended from a plant or cell so altered and which comprises the alteration. As used herein, the term “mutated” or “genetically modified” or “transgenic” or “transformed” or “edited” plants, plant cells, plant tissues, plant parts or seeds refers plants, plant cells, plant tissues, plant parts or seeds that have been mutated by the methods of the present disclosure to include one or more mutations (e.g., insertions, substitutions, or deletions) in the genomic sequence.

As used herein, a “control plant” or “control plant part” or “control cell” or “control seed” refers to a plant or plant part or plant cell or seed that has not been subject to the methods and compositions described herein. A “control” or “control plant” or “control plant part” or “control cell” or “control seed” provides a reference point for measuring changes in phenotype of the subject plant or plant cell. A control plant or plant cell may comprise, for example: (a) a control promoter with reference to the promoters of the present disclosure; (b) a wild-type plant or cell, i.e., of the same genotype as the starting material for the genetic alteration which resulted in the subject plant or cell; (c) a plant or plant cell of the same genotype as the starting material but which has been transformed with a null construct (i.e. with a construct which has no known effect on the trait of interest, such as a construct comprising a marker gene); (d) a plant or plant cell which is a non-transformed segregant among progeny of a subject plant or plant cell; (e) a plant or plant cell genetically identical to the subject plant or plant cell but which is not exposed to conditions or stimuli (e.g., sucrose) that would induce expression of the polynucleotide of interest; or (f) the subject plant or plant cell itself, under conditions in which the polynucleotide of interest is not expressed. In certain instances, a control plant of the present disclosure is grown under the same environmental conditions (e.g., same or similar temperature, humidity, air quality, soil quality, water quality, and/or pH conditions) as a subject plant described herein. Similarly, a control protein or control protein composition can refer to a protein or protein composition that is isolated or derived from a control plant. In specific embodiments, a control plant, plant part, or plant cell is a plant, plant part, or plant cell that comprises a control promoter molecule or does not comprise the promoter molecule of the present disclosure.

Plant cells possess nuclear, plastid, and mitochondrial genomes. The compositions and methods of the present invention may be used to modify the sequence of the nuclear, plastid, and/or mitochondrial genome, or may be used to modulate the expression of a gene or genes encoded by the nuclear, plastid, and/or mitochondrial genome. Accordingly, by “chromosome” or “chromosomal” is intended the nuclear, plastid, or mitochondrial genomic DNA. “Genome” as it applies to plant cells encompasses not only chromosomal DNA found within the nucleus, but organelle DNA found within subcellular components (e.g., mitochondria or plastids) of the cell.

As used herein, the term “gene” or “coding sequence”, herein used interchangeably, refers to a functional nucleic acid unit encoding a protein, polypeptide, or peptide. As will be understood by those in the art, this functional term includes genomic sequences, cDNA sequences, and smaller engineered gene segments that express, or may be adapted to express proteins, polypeptides, domains, peptides, fusion proteins, and mutants.

As used herein, the term a “nucleic acid”, used interchangeably with a “nucleotide”, refers to a molecule consisting of a nucleoside and a phosphate that serves as a component of DNA or RNA. For instance, nucleic acids include adenine, guanine, cytosine, uracil, and thymine.

As used herein, a “mutation” is any change in a nucleic acid sequence. Nonlimiting examples comprise insertions, deletions, duplications, substitutions, inversions, and translocations of any nucleic acid sequence, regardless of how the mutation is brought about and regardless of how or whether the mutation alters the functions or interactions of the nucleic acid. For example, and without limitation, a mutation may produce altered enzymatic activity of a ribozyme, altered base pairing between nucleic acids (e.g. RNA interference interactions, DNA-RNA binding, etc.), altered mRNA folding stability, and/or how a nucleic acid interacts with polypeptides (e.g. DNA-transcription factor interactions, RNA-ribosome interactions, guide RNA-endonuclease reactions, etc.). A mutation might result in the production of proteins with altered amino acid sequences (e.g. missense mutations, nonsense mutations, frameshift mutations, etc.) and/or the production of proteins with the same amino acid sequence (e.g. silent mutations). Certain synonymous mutations may create no observed change in the plant while others that encode for an identical protein sequence nevertheless result in an altered plant phenotype (e.g. due to codon usage bias, altered secondary protein structures, etc.). Mutations may occur within coding regions (e.g., open reading frames) or outside of coding regions (e.g., within promoters, terminators, untranslated elements, or enhancers), and may affect, for example and without limitation, gene expression levels, gene expression profiles, protein sequences, and/or sequences encoding RNA elements such as tRNAs, ribozymes, ribosome components, and microRNAs.

Accordingly, “plant with a mutation” or “plant part with a mutation” or “plant cell with a mutation” or “plant genome with a mutation” refers to a plant or plant part or plant cell or plant genome that contains a mutation (e.g., an insertion, a substitution, or a deletion) described in the present disclosure.

“Genome editing” or “gene editing” as used herein refers to a type of genetic engineering by which one or more mutations (e.g., insertions, substitutions, deletions, modifications) are introduced at a specific location of the genome. “Editing reagents”, as used herein, refers to a set of molecules or a construct comprising or encoding the molecules for introducing one or more mutations in the genome. Exemplary editing reagents comprise a nuclease and a guide RNA. For example, a CRISPR (clustered regularly interspaced short palindromic repeats) system comprises a CRISPR nuclease [e.g., CRISPR-associated (Cas) endonuclease or a variant thereof, such as Cas12a] and a guide RNA. A CRISPR nuclease associates with a guide RNA that directs nucleic acid cleavage by the associated endonuclease by hybridizing to a recognition site in a polynucleotide. The guide RNA comprises a direct repeat and a guide sequence, which is complementary to the target recognition site. In certain embodiments, the CRISPR system further comprises a tracrRNA (trans-activating CRISPR RNA) that is complementary (fully or partially) to the direct repeat sequence present on the guide RNA. A “TALEN” nuclease is an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, and yeast HO endonuclease. A “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, and yeast HO endonuclease.

As used herein, the terms “nuclease” and “endonuclease” are used interchangeably to refer to naturally-occurring or engineered enzymes, which cleave a phosphodiester bond within a polynucleotide chain. The cleavage could be a single strand cleavage or a double strand cleavage. In certain embodiments, the nuclease lacks cleavage activity and is referred to as nuclease dead.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory molecules and polynucleotides that are not found together in nature. For example, a recombinant DNA construct may comprise regulatory molecules and polynucleotides that are derived from different sources, or regulatory molecules and polynucleotides derived from the same source and arranged in a manner different than that found in nature. Such a construct may be used by itself or may be used in conjunction with a vector.

An expression construct can permit transcription of a particular nucleotide sequence in a host cell (e.g., a bacterial cell or a plant cell). An expression cassette may be part of a plasmid, viral genome, or nucleic acid fragment. Typically, an expression cassette includes a polynucleotide to be transcribed, operably linked to a promoter. “Operably linked” refers to a functional linkage between two or more elements. For example, an operable linkage between a promoter of the present invention and a nucleic acid molecule (e.g., native, endogenous, or heterologous nucleic acid molecule) is a functional link that allows for expression of the nucleic acid molecule. Operably linked elements may be contiguous or non-contiguous. When used to refer to the joining of two protein coding regions, by operably linked is intended that the coding regions are in the same reading frame. The cassette may additionally contain at least one additional polynucleotide to be co-transformed into the plant. Alternatively, the additional polynucleotide(s) can be provided on multiple expression cassettes or DNA constructs. Such an expression cassette or construct is provided with a plurality of restriction sites and/or recombination sites for insertion of the heterologous nucleotide sequence of interest to be under the transcriptional regulation of the promoter regions of the invention. The expression cassette may additionally contain selectable marker genes. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

As used herein, “function” of a gene, a polynucleotide, a peptide, a protein, or a molecule refers to activity of a gene, a polynucleotide, a peptide, a protein, or a molecule. For example, the function of a guide RNA or a CRISPR nuclease may be assessed by editing efficiency of a target gene.

As used herein, the term “expression” or “expressing” refers to the transcription and/or translation of a particular nucleic acid sequence driven by a promoter.

“Introduced” in the context of inserting a nucleic acid molecule (e.g., a DNA construct comprising a promoter molecule and a polynucleotide sequence of interest) into a cell, a plant, or a plant part means “transfection” or “transformation” or “transduction” and includes reference to the incorporation of a nucleic acid fragment into a plant cell where the nucleic acid fragment may be incorporated into the genome of the cell (e.g., nuclear chromosome, plasmid, plastid chromosome or mitochondrial chromosome), converted into an autonomous replicon, or transiently expressed (e.g., transfected mRNA).

As used herein with respect to a parameter, the term “decreased” or “decreasing” or “decrease” or “reduced” or “reducing” or “reduce” or “lower” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%) negative change in the parameter from a comparison control, e.g., an established normal or reference level of the parameter, or an established standard control. Accordingly, the terms “decreased”, “reduced”, and the like encompass both a partial reduction and a complete reduction compared to a control.

As used herein with respect to a parameter, the term “increased” or “increasing” or “increase” or “enhanced” or “enhancing” or “enhance” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, or 1000%) positive change in the parameter from a comparison control, e.g., an established normal or reference level of the parameter, or an established standard control.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein, the term “polypeptide” refers to a linear organic polymer containing a large number of amino-acid residues bonded together by peptide bonds in a chain, forming part of (or the whole of) a protein molecule. The amino acid sequence of the polypeptide refers to the linear consecutive arrangement of the amino acids comprising the polypeptide, or a portion thereof.

As used herein the term “polynucleotide” refers to a single or double stranded nucleic acid sequence which is isolated and provided in the form of an RNA sequence (e.g., an mRNA sequence), a complementary polynucleic acid sequence (cDNA), a genomic polynucleic acid sequence and/or a composite polynucleic acid sequences (e.g., a combination of the above).

The term “isolated” refers to at least partially separated from the natural environment e.g., from a plant cell.

As used herein, the terms “exogenous” or “heterologous” in reference to a nucleic acid sequence or amino acid sequence are intended to mean a sequence that is purely synthetic, that originates from a foreign species, or, if from the same species, is substantially modified from its native form in composition and/or genomic locus by deliberate human intervention. Thus, a heterologous nucleic acid sequence may not be naturally expressed within the plant (e.g., a nucleic acid sequence from a different species) or may have altered expression when compared to the corresponding wild type plant. An exogenous polynucleotide may be introduced into the plant in a stable or transient manner, so as to produce a ribonucleic acid (RNA) molecule and/or a polypeptide molecule. It should be noted that the exogenous polynucleotide may comprise a nucleic acid sequence which is identical or partially homologous to an endogenous nucleic acid sequence of the plant.

As used herein, the term “endogenous” in reference to a gene or nucleic acid or protein is intended to mean a gene or nucleic acid or protein that is naturally comprised within or expressed by a cell. Endogenous genes can include genes that naturally occur in the cell of a plant, but that have been modified in the genome of the cell without insertion or replacement of a heterologous gene that is from another plant species or another location within the genome of the modified cell.

As used herein, “fertilization” and/or “crossing” broadly includes bringing the genomes of gametes together to form zygotes but also broadly may include pollination, syngamy, fecundation and other processes related to sexual reproduction. Typically, a cross and/or fertilization occurs after pollen is transferred from one flower to another, but those of ordinary skill in the art will understand that plant breeders can leverage their understanding of fertilization and the overlapping steps of crossing, pollination, syngamy, and fecundation to circumvent certain steps of the plant life cycle and yet achieve equivalent outcomes, for example, a plant or cell of a soybean cultivar described herein. In certain embodiments, a user of this innovation can generate a plant of the claimed invention by removing a genome from its host gamete cell before syngamy and inserting it into the nucleus of another cell. While this variation avoids the unnecessary steps of pollination and syngamy and produces a cell that may not satisfy certain definitions of a zygote, the process falls within the definition of fertilization and/or crossing as used herein when performed in conjunction with these teachings. In certain embodiments, the gametes are not different cell types (i.e. egg vs. sperm), but rather the same type and techniques are used to effect the combination of their genomes into a regenerable cell. Other embodiments of fertilization and/or crossing include circumstances where the gametes originate from the same parent plant, i.e. a “self” or “self-fertilization”. While selfing a plant does not require the transfer pollen from one plant to another, those of skill in the art will recognize that it nevertheless serves as an example of a cross, just as it serves as a type of fertilization. Thus, methods and compositions taught herein are not limited to certain techniques or steps that must be performed to create a plant or an offspring plant of the claimed invention, but rather include broadly any method that is substantially the same and/or results in compositions of the claimed invention.

“Homolog” or “homologous sequence” may refer to both orthologous and paralogous sequences. Paralogous sequence relates to gene-duplications within the genome of a species. Orthologous sequence relates to homologous genes in different organisms due to ancestral relationship. Thus, orthologs are evolutionary counterparts derived from a single ancestral gene in the last common ancestor of given two species and therefore have great likelihood of having the same function. One option to identify homologs (e.g., orthologs) in monocot plant species is by performing a reciprocal BLAST search. This may be done by a first blast involving blasting the sequence-of-interest against any sequence database, such as the publicly available NCBI database which may be found at: ncbi.nlm.nih.gov. If orthologs in rice were sought, the sequence-of-interest would be blasted against, for example, the 28,469 full-length cDNA clones from Oryza sativa Nipponbare available at NCBI. The blast results may be filtered. The full-length sequences of either the filtered results or the non-filtered results are then blasted back (second blast) against the sequences of the organism from which the sequence-of-interest is derived. The results of the first and second blasts are then compared. An ortholog is identified when the sequence resulting in the highest score (best hit) in the first blast identifies in the second blast the query sequence (the original sequence-of-interest) as the best hit. Using the same rational a paralog (homolog to a gene in the same organism) is found. In case of large sequence families, the ClustalW program may be used [ebi.ac.uk/Tools/clustalw2/index.html], followed by a neighbor-joining tree (wikipedia.org/wiki/Neighbor-joining) which helps visualizing the clustering.

In some embodiments, the term “homolog” as used herein, refers to functional homologs of genes. A functional homolog is a gene encoding a polypeptide that has sequence similarity to a polypeptide encoded by a reference gene, and the polypeptide encoded by the homolog carries out one or more of the biochemical or physiological function(s) of the polypeptide encoded by the reference gene. In general, it is preferred that functional homologs and/or polypeptides encoded by functional homologs share at least some degree of sequence identity with the reference gene or polypeptide encoded by the reference gene. Homology (e.g., percent homology, sequence identity+sequence similarity) can be determined using any homology comparison software computing a pairwise sequence alignment.

As used herein, “sequence identity,” “identity,” “percent identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. The determination of percent sequence identity between any two sequences can be accomplished using a mathematical algorithm, or a computer implementation thereof. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms for comparison of sequences to determine sequence identity include, but are not limited to: CLUSTAL in the PC/Gene program (available from Intelligenetics, Mountain View, California); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, California, USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection. Identity (e.g., percent homology) can be determined using any homology comparison software, including for example, the BlastN software of the National Center of Biotechnology Information (NCBI) such as by using default parameters.

According to some embodiments, the identity is a global identity, i.e., an identity over the entire amino acid or nucleic acid sequences of the invention and not over portions thereof.

According to some embodiments, the term “homology” or “homologous” refers to identity of two or more nucleic acid sequences; or identity of two or more amino acid sequences; or the identity of an amino acid sequence to one or more nucleic acid sequence. According to some embodiments, the homology is a global homology, e.g., a homology over the entire amino acid or nucleic acid sequences of the invention and not over portions thereof. The degree of homology or identity between two or more sequences can be determined using various known sequence comparison tools which are described in WO2014/102774.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “population” refers to a set comprising any number, including one, of individuals, objects, or data from which samples are taken for evaluation, e.g., estimating quantitative trait locus (QTL) associations and/or disease tolerance. Most commonly, the terms relate to a breeding population of plants from which members are selected and crossed to produce progeny in a breeding program. A population of plants can include the progeny of a single breeding cross or a plurality of breeding crosses and can be either actual plants or plant derived material, or in silico representations of plants. The member of a population need not be identical to the population members selected for use in subsequent cycles of analyses, nor does it need to be identical to those population members ultimately selected to obtain a final progeny of plants. Often, a plant population is derived from a single biparental cross but can also derive from two or more crosses between the same or different parents. Although a population of plants can comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population in a plant breeding program.

As used herein, the term “crop performance” is used synonymously with “plant performance” and refers to of how well a plant grows under a set of environmental conditions and cultivation practices. Crop performance can be measured by any metric a user associates with a crop's productivity (e.g., yield), appearance and/or robustness (e.g., color, morphology, height, biomass, maturation rate, etc.), product quality (e.g., fiber lint percent, fiber quality, seed protein content, seed carbohydrate content, etc.), cost of goods sold (e.g., the cost of creating a seed, plant, or plant product in a commercial, research, or industrial setting) and/or a plant's tolerance to disease (e.g., a response associated with deliberate or spontaneous infection by a pathogen) and/or environmental stress (e.g., drought, flooding, low nitrogen or other soil nutrients, wind, hail, temperature, day length, etc.). Crop performance can also be measured by determining a crop's commercial value and/or by determining the likelihood that a particular inbred, hybrid, or variety will become a commercial product, and/or by determining the likelihood that the offspring of an inbred, hybrid, or variety will become a commercial product. Crop performance can be a quantity (e.g., the volume or weight of seed or other plant product measured in liters or grams) or some other metric assigned to some aspect of a plant that can be represented on a scale (e.g., assigning a 1-10 value to a plant based on its disease tolerance).

A plant, or its environment, can be contacted with a wide variety of “agriculture treatment agents.” As used herein, an “agriculture treatment agent”, or “treatment agent”, or “agent” can refer to any exogenously provided compound that can be brought into contact with a plant tissue (e.g. a seed) or its environment that affects a plant's growth, development and/or performance, including agents that affect other organisms in the plant's environment when those effects subsequently alter a plant's performance, growth, and/or development (e.g. an insecticide that kills plant pathogens in the plant's environment, thereby improving the ability of the plant to tolerate the insect's presence). Agriculture treatment agents also include a broad range of chemicals and/or biological substances that are applied to seeds, in which case they are commonly referred to as seed treatments and/or seed dressings. Seed treatments are commonly applied as either a dry formulation or a wet slurry or liquid formulation prior to planting and, as used herein, generally include any agriculture treatment agent including growth regulators, micronutrients, nitrogen-fixing microbes, and/or inoculants. Agriculture treatment agents include pesticides (e.g. fungicides, insecticides, bactericides, etc.) hormones (abscisic acids, auxins, cytokinins, gibberellins, etc.) herbicides (e.g. glyphosate, atrazine, 2,4-D, dicamba, etc.), nutrients (e.g. a plant fertilizer), and/or a broad range of biological agents, for example a seed treatment inoculant comprising a microbe that improves crop performance, e.g. by promoting germination and/or root development. In certain embodiments, the agriculture treatment agent acts extracellularly within the plant tissue, such as interacting with receptors on the outer cell surface. In some embodiments, the agriculture treatment agent enters cells within the plant tissue. In certain embodiments, the agriculture treatment agent remains on the surface of the plant and/or the soil near the plant. In certain embodiments, the agriculture treatment agent is contained within a liquid. Such liquids include, but are not limited to, solutions, suspensions, emulsions, and colloidal dispersions. In some embodiments, liquids described herein will be of an aqueous nature. However, in various embodiments, such aqueous liquids that comprise water can also comprise water insoluble components, can comprise an insoluble component that is made soluble in water by addition of a surfactant, or can comprise any combination of soluble components and surfactants. In certain embodiments, the application of the agriculture treatment agent is controlled by encapsulating the agent within a coating, or capsule (e.g. microencapsulation). In certain embodiments, the agriculture treatment agent comprises a nanoparticle and/or the application of the agriculture treatment agent comprises the use of nanotechnology. In some embodiments, the plants described herein can grow in the presence of one or more agricultural treatment agents. For example, the plants described herein can have an increased expression of the polynucleotide of interest, e.g., a guide RNA or a nuclease, or mutations in the genome introduced by such editing reagents, and can grow in the presence of commonly used herbicides.

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

All publications, patent applications, patents, and other references mentioned herein are incorporated by reference herein in their entirety.

### II. Overview of the Invention

Enhanced expression of a polynucleotide of interest in a plant or plant part can promote successful development of desirable traits. For instance, the efficiency of introducing mutations can be closely associated with expression levels and spatio-temporal expression patterns of reagents that are responsible for introducing a mutation (e.g., editing reagents). For example, following delivery of expression constructs, expression determines the level of bioavailable ribonucleoprotein complexes for introducing mutations using a CRISPR system. Methods and compositions for increasing CRISPR reagent expression have the potential to drive significant improvements in plant editing efficiencies, and types of mutation outcomes (such as homology-directed repair).

Accordingly, efficient promoters for expressing a polynucleotide of interest in plant cells are advantageous. Provided herein are compositions and methods for expressing a polynucleotide of interest in plants, including in a legume embryonic axes. The compositions and methods described herein can enable routine high-level editing of crop plants, by targeting plant tissues including the meristems. The meristems of the explants a desirable target of transforming editing reagents as the meristems undergo editing concurrent with their growth into whole plants. Further, the compositions and methods described herein can enable regenerated plants to contain a ubiquitous (heritable) editing outcome, rather than a mosaic pattern of editing events across somatic tissues or no detectable editing in TO plants. The promoter and leader elements can have an immediate, efficient induction of expression of operably linked editing reagents in transformed meristems to enable editing to at a rate faster than their cellular division.

Described herein are promoters, including 5′ untranslated regions (5′UTRs), for efficient expression and function of downstream polynucleotides of interest in a plant or plant part. Provided herein are novel pea (Pisum sativum) ELONGATION FACTOR 1-ALPHA promoters (PsEF1Ap, e.g., PsEF1A-0p, PsEF1A-1p, PsEF1A-6p, PsEF1A-7p) and PsEF1A promoter homologs, and variants and fragments of any thereof, that can enhance expression of operably linked polynucleotides of interest compared to control promoters (e.g., AtUBI11p). PsEF1A promoter homologs can include AhEF1Ap homologs (arahy.Tifrunner.gnm1.ann1.S49I6H, . . . PSFB2E, . . . 08YRK9, . . . G03TES), CaEF1Ap homologs (Ca_10593 . . . 10594, . . . 11433, . . . 14697, . . . 14698, . . . 22296), LaEF1Ap homologs (La1b_Chr02g0141951, . . . Chr02g0158111, . . . Chr06g0174461, . . . Chr14g0362911, . . . Chr19 g0129561, . . . Chr21g0317711, . . . Chr24g0403471), LjEF1Ap homologs (Lj1g0010515, Lj1g0026760, Lj2g0002755, Lj2g0008477, Lj5g0021169), MtEF1Ap homologs (Medtr4g014810, Medtr6g021800, Medtr6g021805, Medtr1g013680), PvEF1A-4Ap (Phvu1.004G060000), PvEF1A-4Bp (Phvu1.004G075100), PvEF1Ap (Phvu1.007G092500), TpEF1Ap homologs (Tp57577_TGAC_v2_gene1766, Tp57577_TGAC_v2_gene5210, Tp57577_TGAC_v2_gene21632, Tp57577_TGAC_v2_gene21641, Tp57577_TGAC_v2_gene24200), and VuEF1Ap homologs (Vigun04g088900, Vigun04g096500, Vigun07g204100, Vigun07g204200). When operably linked to editing reagents, PsEF1Ap and other PsEF1A promoter homologs can enhance expression of editing reagents and editing frequency at target sites compared to control promoters.

The promoter molecules of the present invention can be useful for expressing operably linked polynucleotides of interest, e.g., in a constitutive manner and/or universally in any tissue in a plant or plant part, including but not limited to meristematic tissue. When operably linked to editing reagents (e.g., CRISPR-Cas12a reagents), the promoter molecules of the present invention can provide high frequencies of heritable genome editing in plants or plant parts.

### III. Nucleic Acid Molecules Comprising a Promoter Sequence

The present disclosure provides promoters, including 5′ untranslated regions (5′UTRs), for expression of downstream polynucleotides of interest in a plant or plant part. As used herein, “promoter” refers to an upstream regulatory region of DNA prior to the ATG of a native gene, having a transcription initiation function for said gene and other downstream genes. A promoter sequence can include a 5′ untranslated region (5′UTR), including intronic sequences, an exon sequence from a coding region, and/or an intron sequence from a coding region in addition to a core promoter that contains a TATA box capable of directing RNA polymerase II (pol II) to initiate RNA synthesis at the appropriate transcription initiation site for a particular polynucleotide sequence of interest. A promoter may additionally comprise other recognition sequences positioned upstream of the TATA box, and well as within the 5′UTR intron, which influence the transcription initiation rate.

As used herein “transcription initiation” refers to a phase during which the first nucleotides in the RNA chain are synthesized. Transcription initiation is a multistep process that starts with formation of a complex between an RNA polymerase holoenzyme and a DNA template at the promoter, and ends with dissociation of the core polymerase from the promoter after the synthesis of approximately first nine nucleotides.

The present disclosure provides nucleic acid molecules comprising a promoter molecule comprising a nucleic acid sequence that shares at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with a nucleic acid sequence of a PsEF1A promoter or a homolog thereof, and retains transcription initiation function. The promoter molecules can comprise a nucleic acid sequence of a PsEF1A promoter or a homolog thereof. The PsEF1A promoter homologs can be PsEF1Ap homologs (e.g., PsEF1A-0p, PsEF1A-1p PsEF1A-6p PsEF1A-7p), AhEF1Ap homologs, CaEF1Ap homologs, LaEF1Ap homologs, LjEF1Ap homologs, MtEF1Ap homologs, PvEF1A-4Ap, PvEF1A-4Bp, PvEF1Ap homologs, TpEF1Ap homologs, and VuEF1Ap homologs, e.g., any one of SEQ ID NOs: 1-4 and 9-46. Ps, Ah, Ca, La, Lj, Mt, Pa, Pv, Vu, At, and Gm represent molecules derived from Pisum sativum (pea), Arachis hypogaea (Peanut), Cicer arietinum (Chickpea), Lupinus albus (White lupin), Lotus japonicus, Medicago truncatula (Barrelclover), Phaseolus vulgaris (Common bean), Trifolium pratense (red clover), Vigna unguiculata (Cowpea), Arabidopsis thaliana, and Glycine max respectively. The promoter molecules of the present disclosure can comprise a nucleic acid sequence that shares at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with a nucleic acid sequence of any one of SEQ ID NOs: 1-4, 9-39, and 43-46, and retains transcription initiation function. The promoter molecules can comprise a nucleic acid sequence of any one of SEQ ID NOs: 1-4, 9-39, and 43-46. In some embodiments, the promoter sequence can comprise the nucleic acid sequence of PsEF1A-0p, PsEF1A-1p, PsEF1A-6p, PsEF1A-7p, which are set forth as SEQ ID NOs: 1-4, respectively. The promoter sequence can share at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-4, and retain transcription initiation function.

Also provided herein are nucleic acid molecules comprising a promoter sequence that comprises one or more deletions, substitutions, and/or insertions) relative to the nucleic acid sequence of a PsEF1A promoter or a homolog thereof (which can include a 5′UTR, a 5′UTR intron, an exon sequence from a coding region, and/or an intron sequence from a coding region). The deletion, substitution, and/or insertion can be synthetic. A “synthetic” deletion, substitution, or insertion in a nucleic acid sequence, as used herein, refers to a deletion, substitution, or insertion that is not naturally occurring and has been introduced to a wild-type (e.g., native, naturally occurring) nucleic acid sequence. The PsEF1A promoter homologs can be PsEF1Ap homologs (e.g., PsEF1A-0p, PsEF1A-1p PsEF1A-6p PsEF1A-7p), AhEF1Ap homologs, CaEF1Ap homologs, LaEF1Ap homologs, LjEF1Ap homologs, MtEF1Ap homologs, PvEF1A-4Ap, PvEF1A-4Bp, PvEF1Ap homologs, TpEF1Ap homologs, and VuEF1Ap homologs, e.g., any one of SEQ ID NOs: 1-4 and 9-46. The promoter sequence can share at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-4 and 9-39, and 43-46, and retain transcription initiation function.

In some embodiments, the promoter sequence (which can include a 5′UTR, a 5′UTR intron, an exon sequence from a coding region, and/or an intron sequence from a coding region) comprises one or more deletions, substitutions, and/or insertions (e.g., synthetic deletions, substitutions, and/or insertions) relative to the nucleic acid sequence of PsEF1A-0p, PsEF1A-1p, PsEF1A-6p, PsEF1A-7p, which are set forth as SEQ ID NOs: 1-4, respectively. The promoter sequence can share at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-4, and retain transcription initiation function.

In the nucleic acid molecule comprising a promoter sequence provided herein, said one or more deletions, substitutions, and/or insertions (e.g., synthetic deletions, substitutions, and/or insertions) can be made to avoid structurally-repetitive sequences such as a microsatellite sequence (a repetitive nucleotide sequence usually several base pairs in length, e.g., GATGATGAT), a sequence duplication, a palindrome, a homo-polymetric tract [e.g., poly(A), poly(T)], to enhance the promoter stability, function, synthesis, or use thereof, and/or to enhance the expression of an operably linked polynucleotide. The one or more deletions, substitutions, and/or insertions (e.g., synthetic deletions, substitutions, and/or insertions) can be made in a 3′ region of said promoter sequence and/or a 5′UTR, a 5′UTR intron, an exon sequence from a coding region, and/or an intron sequence from a coding region of the promoter sequence. A “3′ region of a promoter sequence”, as used herein, refers to a region located toward the 3′ end in a promoter sequence and can be located in the 5′ UTR or 5′ intron sequences of the downstream gene. A 3′ region of a promoter sequence is more proximal than a 5′ region of the promoter sequence to the downstream gene or operably linked polynucleotide. For example, the promoter sequence can share at least 80% sequence identity with any one of SEQ ID NOs: 5-8, or comprise the nucleic acid sequence of any one of SEQ ID NOs: 5-8, and retain transcription initiation function.

The promoters of the invention can be used to express or enhance expression of any nucleic acid molecule of interest, such as any gene, polynucleotide, or regulatory element of interest. Eukaryotic promoters are complex and are comprised of components that include a TATA box consensus sequence at about 35 base pairs 5′ relative to the transcription start site or cap site which is defined as +1. The TATA motif is the site where the TATA-binding-protein (TBP) as part of a complex of several polypeptides (TFIID complex) binds and productively interacts (directly or indirectly) with factors bound to other sequence elements of the promoter. This TFIID complex in turn recruits the RNA polymerase II complex to be positioned for the start of transcription generally 25 to 30 base pairs downstream of the TATA element and promotes elongation thus producing RNA molecules. The sequences around the start of transcription (designated INR) of some pol I genes seem to provide an alternate binding site for factors that also recruit members of the TFIID complex and thus “activate” transcription. These INR sequences are particularly relevant in promoters that lack functional TATA elements providing the core promoter binding sites for eventual transcription. It has been proposed that promoters containing both a functional TATA and INR motif are the most efficient in transcriptional activity. (Zenzie-Gregory et al, 1992. J. Biol. Chem. 267:2823-2830). See, for example, U.S. Pat. No. 6,072,050, herein incorporated by reference.

The invention encompasses isolated or substantially purified polynucleotide or nucleic acid compositions. An “isolated” or “purified” polynucleotide, or biologically active portion thereof, is substantially or essentially free from components that normally accompany or interact with the polynucleotide as found in its naturally occurring environment. Thus, an isolated or purified polynucleotide is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. Fragments and variants of the disclosed promoter molecules are also encompassed by the present invention. By “fragment” is intended a portion of the nucleic acid sequence. Variant sequences can be isolated by PCR as well as hybridization. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, New York). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

The promoters of the invention can have a number of characteristics. The promoter may have a constitutive expression profile. In some aspects, the promoters of the present disclosure provide constitutive expression of an operably linked nucleotide of interest (e.g., encoding a guide RNA or nuclease). In some aspects, the promoters of the present disclosure provide increased constitutive expression of an operably linked polynucleotide of interest (e.g., guide RNA, nuclease) compared to a control promoter. The promoters of the present disclosure can have superior effects in expressing a polynucleotide of interest in a plant or plant part compared to constitutive promoters known in the art, e.g., the CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J 3:2723-2730); ALS promoter (U.S. Pat. No. 5,659,026), and the like.

Additionally or alternatively, the promoters of the present disclosure can be tissue-specific promoters. A “tissue specific” promoter is a promoter that initiates transcription only in certain tissues, such as leaves, roots, fruit, seeds, or flowers. Unlike constitutive expression of genes, tissue-specific expression is the result of several interacting levels of gene regulation. As such, promoters from homologous or closely related plant species can be preferable to use to achieve efficient and reliable expression of transgenes in particular tissues. In some embodiments, the promoters of the present disclosure can be tissue-preferred promoters. A “tissue preferred” promoter is a promoter that initiates transcription mostly, but not necessarily entirely or solely in certain tissues. In some embodiments, the promoters of the present disclosure preferably target meristematic tissue. The promoters of the present disclosure can have superior effects in expressing a polynucleotide of interest in a plant or plant part compared to tissue-specific or tissue-preferred promoters described in the art, e.g., Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2): 157-168; Rinehart et al. (1996) Plant Physiol. 112(3): 1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Leaf-preferred promoters are also known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mot Biol. 23(6):1129-1138; and Matsuoka et al. (1993) Proc. Natl. Acad. Sci. USA 90(20):9586-9590.

Additionally or alternatively, promoters of the present disclosure can be cell type specific promoters or cell type preferred promoters. A “cell type specific” promoter is a promoter that primarily drives expression in certain cell types in one or more organs, for example, embryonic tissue cells. A “cell type preferred” promoter is a promoter that primarily drives expression mostly, but not necessarily entirely or solely in certain cell types in one or more organs, for example, embryonic cells. Such promoters may primarily or preferentially drive the expression of a downstream polynucleotide in a particular cell type such as a meristematic tissue cell. The promoters of the present disclosure can have superior effects in expressing a polynucleotide of interest in a plant or plant part compared to cell type specific or cell type preferred promoters described in the art, e.g., Viret et al. (1994) Proc Natl Acad USA 91: 8577-8581; U.S. Pat. Nos. 8,455,718; 7,642,347; Sattarzadeh et al. (2010) Plant Biotechnol J 8: 112-125; Engelmann et al. (2008) Plant Physiol 146: 1773-1785; Matsuoka et al. (1994) Plant J6: 311-319, and the like.

Additionally or alternatively, promoters of the present disclosure can be developmentally-regulated promoters. Such promoters may show a peak in expression at a particular developmental stage. The promoters of the present disclosure can have superior effects in expressing a polynucleotide of interest in a plant or plant part compared to developmentally-regulated promoters described in the art, e.g., U.S. Pat. No. 10,407,670; Gan and Amasino (1995) Science 270: 1986-1988; Rinehart et al. (1996) Plant Physiol 112: 1331-1341; Gray-Mitsumune et al. (1999) Plant Mol Biol 39: 657-669; Beaudoin and Rothstein (1997) Plant Mol Biol 33: 835-846; Genschik et al. (1994) Gene 148: 195-202, and the like.

Additionally or alternatively, promoters of the present disclosure can be promoters that are induced following the application of a particular biotic and/or abiotic stress. The promoters of the present disclosure can have superior effects in expressing a polynucleotide of interest in a plant or plant part compared to inducible promoters described in the art, e.g., Yi et al. (2010) Planta 232: 743-754; Yamaguchi-Shinozaki and Shinozaki (1993) Mol Gen Genet 236: 331-340; U.S. Pat. No. 7,674,952; Rerksiri et al. (2013) Sci World J2013: Article ID 397401; Khurana et al. (2013) PLoS One 8: e54418; Tao et al. (2015) Plant Mol Biol Rep 33: 200-208, and the like.

It is recognized that, in some instances, a specific, non-constitutive expression profile may provide an improved plant phenotype. For instance, many plant genes are regulated by light conditions, the application of particular stresses, the circadian cycle, or the stage of a plant's development. These expression profiles may be important for the function of the gene, polynucleotide, gene product, or polynucleotide product in planta. One strategy that may be used to provide a desired expression profile in combination with the promoters, compositions, or methods of the present disclosure is the use of synthetic promoters containing cis-regulatory elements that drive the desired expression levels at the desired time and place in the plant. The promoters of the present disclosure can comprise cis-regulatory elements that can be used to alter polynucleotide expression in planta. Further, the promoters of the present disclosure can have superior effects in expressing a polynucleotide of interest in a plant or plant part compared to promoters comprising cis-regulatory elements that have been described in the scientific literature, e.g., Vandepoele et al. (2009) Plant Physiol 150: 535-546; Rushton et al. (2002) Plant Cell 14: 749-762). Cis-regulatory elements may also be used to alter promoter expression profiles, as described in Venter (2007) Trends Plant Sci 12: 118-124.

### IV. Constructs Comprising a Promoter Operably Linked to a Polynucleotide of Interest

The present disclosure provides DNA constructs (e.g., expression constructs) comprising, in operable linkage, the promoter molecule of the present disclosure and a polynucleotide of interest comprising a nucleotide sequence of interest (e.g., encoding any polynucleotide of interest, such as a guide RNA, a nuclease, or a selectable marker). The DNA construct can include one promoter molecule operably linked to one or more polynucleotides of interest. Alternatively, the DNA construct can include more than one promoter molecules each operably linked to one or more polynucleotides of interest, wherein at least one of the promoter molecules are a promoter molecule of the present disclosure (e.g., a PsEF1A promoter, a homolog, a fragment, or a variant thereof).

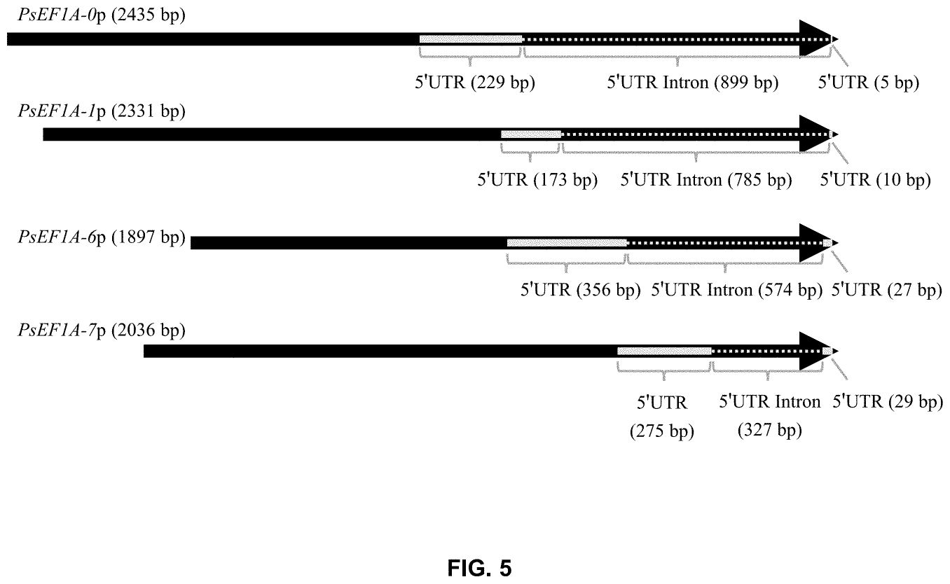

The promoter molecule of the DNA construct can comprise a nucleic acid sequence that shares at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with a nucleic acid sequence of a PsEF IA promoter or a homolog thereof, and retains transcription initiation function. The promoter molecules can comprise a nucleic acid sequence of a PsEF1A promoter or a homolog thereof. The PsEF1A promoter homologs can be PsEF1Ap homologs (e.g., PsEF1A-0p, PsEF1A-1p PsE1A-6p PsEF1A-7p), AhEF1Ap homologs, CaEF1Ap homologs, LaEF1Ap homologs, LjEF1Ap homologs, MtEF1Ap homologs, PvEF1A-4Ap, PvEF1A-4Bp, PvEF1Ap homologs, TpEF1Ap homologs, and VuEF1Ap homologs, e.g., any one of SEQ ID NOs: 1-4 and 9-46. The promoter molecules of the present disclosure can comprise a nucleic acid sequence that shares at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with a nucleic acid sequence of any one of SEQ ID NOs: 1-4, 9-39, and 43-46, and retains transcription initiation function. The promoter molecules can comprise a nucleic acid sequence of any one of SEQ ID NOs: 1-4, 9-39, and 43-46. In some embodiments, the promoter molecule of the DNA construct can comprise the nucleic acid sequence of PsEF1 A-0p, PsEF1A-1p, PsEF1A-6p, PsEF1A-7p, which are set forth as SEQ ID NOs: 1-4, respectively. The sequence diagrams of PsEF1A-0p, PsEF1A-1p, PsEF1A-6p, PsEF IA-7p are also schematically depicted in FIG. 5. The promoter sequence can share at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-4, and retain transcription initiation function.

The promoter molecule of the DNA construct can also comprise a promoter sequence having one or more deletions, substitutions, and/or insertions (e.g., synthetic deletions, substitutions, and/or insertions) relative to the nucleic acid sequence of a PsEF1A promoter or a homolog thereof (which can include a 5′UTR, a 5′UTR intron, an exon sequence from a coding region, and/or an intron sequence from a coding region). Said one or more deletions, substitutions, and/or insertions (e.g., synthetic deletions, substitutions, and/or insertions) can be directed to structurally-repetitive sequences such as a microsatellite sequence, a sequence duplication, a palindrome, a homo-polymetric tract, to enhance the promoter stability, function, synthesis, or use thereof, and/or to enhance the expression of an operably linked polynucleotide. The one or more deletions, substitutions, and/or insertions (e.g., synthetic deletions, substitutions, and/or insertions) can be made in a 3′ region of said promoter sequence and/or a 5′UTR, a 5′UTR intron, an exon sequence from a coding region, and/or an intron sequence from a coding region of the promoter sequence. The PsEF1A promoter homologs can be PsEF1Ap homologs (e.g., PsEF1A-0p, PsEF1A-1p PsEF1A-6p PsEF1A-7p), AhrF1Ap homologs, CaEF1Ap homologs, LaEF1Ap homologs, LjEF1Ap homologs, MtEF1Ap homologs, PvEF1A-4Ap, PvEF1A-4Bp, PvEF1Ap homologs, TpEF1Ap homologs, and VuEF1Ap homologs, e.g., any one of SEQ ID NOs: 1-4 and 9-46. The promoter sequence can share at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-4 and 9-39, and 43-46, and retain transcription initiation function. In some embodiments, the promoter sequence (which can include a 5′UTR, a 5′UTR intron, an exon sequence from a coding region, and/or an intron sequence from a coding region) comprises one or more deletions, substitutions, and/or insertions (e.g., synthetic deletions, substitutions, and/or insertions) relative to the nucleic acid sequence of PsEF1A-0p, PsEF1A-1p, PsEF1A-6p, PsEF1A-7p, which are set forth as SEQ ID NOs: 1-4, respectively. The promoter sequence can share at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-4, and retain transcription initiation function. For example, the promoter sequence can share at least 80% sequence identity with any one of SEQ ID NOs: 5-8, or comprise the nucleic acid sequence of any one of SEQ ID NOs: 5-8, and retain transcription initiation function.

### A. Polynucleotides of Interest Operably Linked to the Promoter Molecule

The promoter molecules disclosed herein can be operably linked to any polynucleotide of interest. As used herein, the term “polynucleotide of interest” can be interchangeable with the terms “coding sequence” or “nucleotide sequence of interest”. For example, the coding sequence(s) may encode editing reagents (e.g., a guide RNA, a nuclease) targeting any gene or genomic site of interest, regulatory RNA, a selectable marker/reporter, an enzyme, a transcription factor, a receptor, a ligand, a molecule that confers resistance to pests or disease, tolerance to herbicides, and/or advantageous agronomic traits (e.g., yield improvement, nitrogen use efficiency, water use efficiency, and nutritional quality). Exemplary polynucleotides of interest to be included in the DNA constructs are described below. However, the present disclosure is not limited to exemplary polynucleotides of interest that can be operably linked to the promoter molecule or DNA constructs of the present disclosure discussed herein.

### 1. Editing Reagents

Polynucleotides of interest can include editing reagents for editing any gene or genomic site of interest. As used herein, “editing reagents” refer to a set of molecules or a construct comprising or encoding the molecules for introducing one or more mutations in the genome, including a nuclease and a guide RNA. For example, editing reagents can be CRISPR reagents, TALEN reagents, and ZFN reagents. A nuclease can be a nickase, an endonuclease, a meganuclease, or a nuclease fusion. CRISPR reagents comprise a CRISPR nuclease (e.g., Cas endonuclease or a variant thereof, such as Cas12a) and a guide RNA. In certain embodiments, the CRISPR components further comprise a tracrRNA (trans-activating CRISPR RNA) that is complementary (fully or partially) to the direct repeat sequence present on the guide RNA. A “TALEN” nuclease is an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, and yeast HO endonuclease. A “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, and yeast HO endonuclease. Editing reagents can also include base editing components. For example, cytosine base editing (CBE) reagents, which change a C-G base pair to a T-A base pair, comprise a single guide RNA, a nuclease (e.g., dCas9, CAS9 nickase), a cytidine deaminase (e.g., APOBEC1), and a uracil DNA glycosylase inhibitor (UGI). Adenine base editing (ABE) reagents, which change an A-T base pair to a G-C base pair comprise a deaminase, (TadA), a nuclease (e.g., dCas or Cas nickase), and a guide RNA.

The promoters of the present disclosure may be operably linked to nuclease sequences. The DNA constructs of the present disclosure may comprise nuclease sequences. Nucleases that can be used in the present disclosure in precise genome-editing technologies to modulate the expression of the endogenous sequence include, but are not limited to, CRISPR nucleases, including Cas9, Cas12a (Cpf1), Cms1 or any CRISPR endonuclease, including CRISPR nickases and nuclease-dead CRISPR nucleases (e.g., a deactivated Cas9, Cas12a, or Cms1 endonuclease); meganucleases designed against the plant genomic sequence of interest (D′Halluin et al (2013) Plant Biotechnol J 11: 933-941); transcription activator-like effector nucleases (TALENs); or zinc finger nucleases (ZFNs).

In some embodiments, the nuclease encoded by the coding sequence of the DNA construct is a clustered, regularly interspaced, short palindromic repeats (CRISPR)-associated Cas endonuclease. In specific embodiments, the CRISPR nuclease is a Cas12a nuclease, herein used interchangeably with a Cpf1 nuclease. In a specific embodiment, the Cas12a nuclease is a McCpf1 nuclease, e.g., a Mc.2Cpf1 2C-NLS (nuclear localization sequence) nuclease. In some embodiments, the CRISPR nuclease shares at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with SEQ ID NO: 62 and retains nuclease activity, or comprises the sequence of SEQ ID NO: 62. In some embodiments, the nuclease is further operably linked to one or more nuclear localization sequences (NLSs) and/or one or more epitope tags. A “nuclear localization sequence (NLS)”, as used herein, refers to a peptide sequence that, when included in a protein sequence, act as a signal fragment to mediate the transport of the protein from the cytoplasm into the nucleus. Different nuclear localized proteins may share the same NLS. Any NLS or epitope tag can be linked to the nuclease provided herein. For example, the nuclease can be linked to a SV40-nucleophosmin 2 (SV40-NPM2) sequence. In specific embodiments, the nuclease can be linked to one or more NLS, such as 1, 2, 3, 4, 5, 6, 7, 8, 9, or 10. The NLS can be located at the N-terminus and/or the C-terminus of the nuclease (5′ and/or 3′ end of the nuclease coding sequence). In some embodiments multiple NLSs are located at each end of the nuclease, such as 2 NLSs at the N-terminus and 2 NLSs at the C-terminus, or 2 NLSs at the N-terminus and 4 NLSs at the C-terminus, or 4 NLSs at the N-terminus and 2-NLSs at the C-terminus. In some embodiments, the CRISPR nuclease with NLS shares at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with SEQ ID NO: 63 and retains the nuclease activity and the NLS activity, or comprises the sequence of SEQ ID NO: 63.

The promoters of the present disclosure may be operably linked to coding sequences for guide RNAs. The DNA constructs of the present disclosure may comprise coding sequences for guide RNAs. To introduce one or more mutations into a gene or a genomic site of interest, antisense constructions, complementary to at least a portion of the messenger RNA (mRNA) for the sequences of the gene or the genomic site of interest can be constructed. Antisense nucleotides are designed to hybridize with the corresponding mRNA. Modifications of the antisense sequences may be made as long as the sequences hybridize to and interfere with expression of the corresponding mRNA. In this manner, antisense constructions having at least 75%, optimally 80%, more optimally 85%, 90%, 95% or greater sequence identity to the corresponding sequences to be edited may be used. Furthermore, portions of the antisense nucleotides may be used to disrupt the expression of the target gene.

In some instances, a guide RNA may comprise a targeting region that is complementary to a targeted sequence as well as another region that allows the guide RNA to form a complex with a nuclease (e.g., a CRISPR nuclease) of interest. The targeting region of a guide RNA for use in the method described herein above may be 10-40 nucleotides long (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides long). For example, the targeting region of a guide RNA for use provided herein may be 24 nucleotides in length.

In some embodiments, the polynucleotide of interest of the DNA construct encodes a guide RNA or a nuclease. In some embodiments, the DNA construct comprises one or more polynucleotides of interest encoding a guide RNA and a nuclease, both operably linked to the same promoter molecule of the DNA construct. In some embodiments, a guide RNA and a nuclease can be each operably linked to different promoter molecules, at least one of which is a promoter molecule of the present disclosure (e.g., a PsEF1Ap, a homolog, a fragment, or a derivative thereof e.g., with one or more deletions, substitutions, and/or insertions).

In some embodiments, the polynucleotide of interest of the DNA construct operably linked to the promoter molecule encodes a guide RNA, and the DNA construct further comprises, in operable linkage, a nucleic acid molecule encoding a second promoter molecule and a polynucleotide encoding a nuclease. In some embodiments, the second promoter molecule is an RNA polymerase II promoter molecule. As used herein, “RNA polymerase II” is an enzyme localized in the nucleoplasm and synthesizes precursors to mRNAs and some small nuclear RNAs (e.g., sRNAs, microRNAs). The second promoter molecule operably linked to a nuclease can comprise a nucleic acid sequence that (i) comprises one or more deletions, substitutions, and/or insertions relative to the nucleic acid sequence of any one of SEQ ID NOs: 1-4, 9-39, and 43-46, shares at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%) sequence identity with any one of SEQ ID NOs: 1-4, 9-39, and 43-46, and retains transcription initiation function, (ii) has at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%) sequence identity to any one of SEQ ID NOs: 1-4, 9-39, 43-52, and 56-61, and retains transcription initiation function, or (iii) is any one of SEQ ID NOs: 1-4, 9-39, 43-52, and 56-61.

In some embodiments, the polynucleotide of interest of the DNA construct operably linked to the promoter molecule encodes a nuclease, and the DNA construct further comprises, in operable linkage: a nucleic acid molecule encoding a second promoter molecule and a polynucleotide encoding a guide RNA. In some embodiments, the second promoter molecule is an RNA polymerase III (pol III) promoter molecule. As used herein, “RNA polymerase III” is an enzyme that transcribes 5S rRNA, tRNA, and some small nuclear RNA genes in the nucleus and cytosol. The second promoter molecule operably linked to a guide RNA can comprise a nucleic acid sequence that (i) comprises one or more deletions, substitutions, and/or insertions relative to the nucleic acid sequence of any one of SEQ ID NOs: 1-4, 9-39, and 43-46, shares at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%) sequence identity with any one of SEQ ID NOs: 1-4, 9-39, and 43-46, and retains transcription initiation function, (ii) has at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%) sequence identity to any one of SEQ ID NOs: 1-4, 47-49, 51, and 53, and retains transcription initiation function, or (iii) is any one of SEQ ID NOs: 1-4, 47-49, 51, and 53.

In embodiments in which the DNA construct comprises polynucleotides of interest encoding a guide RNA and a nuclease operably linked to the same promoter or different promoters, exemplary combinations of promoters operably linked to polynucleotides encoding a nuclease and a guide RNA are indicated with “X” in Table 1 below. Each promoter in Table 1 is meant to include its variants and fragments, e.g., promoter molecules that share at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence similarity with the promoter molecule indicated, including promoter molecules comprising one or more deletions, substitutions, and/or insertions (e.g., synthetic deletions, substitutions, and/or insertions) thereof and retaining transcription initiation function. Combinations are not limited to those listed herein. Any other sequences and combinations of promoters may be included in the DNA constructs, in addition to any variations to other components of the DNA constructs, according to the present disclosure.

TABLE 1
Exemplary Promoters for Nuclease and Guide RNA Coding Sequences

| | | Promoter Operably Linked to Nuclease | | | | | | | | |
|---|---|---|---|---|---|---|---|---|---|---|
| | | | | | | PsEF1Ap | | | | |
| | | | | PsEF1A- | PsEF1A- | homologs | | | PsUBI3- | |
| | | PsEF1A-0p | PsEF1A-1p | 6p | 7p | (SEQ ID | AtUBI11p | PsUBI3p | SYN3p | SIUBI7p |
| | | (SEQ ID | (SEQ ID | (SEQ ID | (SEQ ID | NO: 9-39, | (SEQ ID | (SEQ ID | (SEQ ID | (SEQ ID |
| | | NO: 1) | NO: 2) | NO: 3) | NO: 4) | 43-46) | NO: 47) | NO: 48) | NO: 49) | NO: 50) |
| Promoter | PsEF1A-0p | X | X | X | X | X | X | X | X | X |
| Operably | (SEQ ID NO: 1) | | | | | | | | | |
| Linked | PsEF1A-1p | X | X | X | X | X | X | X | X | X |
| to Guide | (SEQ ID NO: 2) | | | | | | | | | |
| RNA | PsEF1A-6p | X | X | X | X | X | X | X | X | X |
| | (SEQ ID NO: 3) | | | | | | | | | |
| | PsEF1A-7p | X | X | X | X | X | X | X | X | X |
| | (SEQ ID NO: 4) | | | | | | | | | |
| | PsEF1Ap | X | X | X | X | X | X | X | X | X |
| | homologs | | | | | | | | | |
| | (SEQ ID NO: | | | | | | | | | |
| | 9-39, 43-46) | | | | | | | | | |
| | AtUBI11p | X | X | X | X | X | | | | |
| | (SEQ ID | | | | | | | | | |
| | NO: 47) | | | | | | | | | |
| | PsUBI3p | X | X | X | X | X | | | | |
| | (SEQ ID | | | | | | | | | |
| | NO: 48) | | | | | | | | | |
| | PsUBI3-SYN3p | X | X | X | X | X | | | | |
| | (SEQ ID | | | | | | | | | |
| | NO: 49) | | | | | | | | | |
| | GmScreamM4p | X | X | X | X | X | | | | |
| | (SEQ ID | | | | | | | | | |
| | NO: 51) | | | | | | | | | |
| | AtU6-26p | X | X | X | X | X | | | | |
| | (SEQ ID | | | | | | | | | |
| | NO: 53) | | | | | | | | | |
| | | | Promoter Operably Linked to Nuclease | | | | | | | |
| | | | | | | | GmYAO- | | | |
| | | | GmScreamM4p | 35S-ENp | AtUBI10p | GmUBIp | 1p | AtRPS5Ap | GmDMC1p | GmLAT52Lp |
| | | | (SEQ ID | (SEQ ID | (SEQ ID | (SEQ ID | (SEQ ID | (SEQ ID | (SEQ ID | (SEQ ID |
| | | | NO: 51) | NO: 52) | NO: 56) | NO: 57) | NO: 58) | NO: 59) | NO: 60) | NO: 61) |
| | Promoter | PsEF1A-0p | X | X | X | X | X | X | X | X |
| | Operably | (SEQ ID NO: 1) | | | | | | | | |
| | Linked | PsEF1A-1p | X | X | X | X | X | X | X | X |
| | to Guide | (SEQ ID NO: 2) | | | | | | | | |
| | RNA | PsEF1A-6p | X | X | X | X | X | X | X | X |
| | | (SEQ ID NO: 3) | | | | | | | | |
| | | PsEF1A-7p | X | X | X | X | X | X | X | X |
| | | (SEQ ID NO: 4) | | | | | | | | |
| | | PsEF1Ap | X | X | X | X | X | X | X | X |
| | | homologs | | | | | | | | |
| | | (SEQ ID NO: | | | | | | | | |
| | | 9-39, 43-46) | | | | | | | | |
| | | AtUBI11p | | | | | | | | |
| | | (SEQ ID | | | | | | | | |
| | | NO: 47) | | | | | | | | |
| | | PsUBI3p | | | | | | | | |
| | | (SEQ ID | | | | | | | | |
| | | NO: 48) | | | | | | | | |
| | | PsUBI3-SYN3p | | | | | | | | |
| | | (SEQ ID | | | | | | | | |
| | | NO: 49) | | | | | | | | |
| | | GmScreamM4p | | | | | | | | |
| | | (SEQ ID | | | | | | | | |
| | | NO: 51) | | | | | | | | |
| | | AtU6-26p | | | | | | | | |
| | | (SEQ ID | | | | | | | | |
| | | NO: 53) | | | | | | | | |

In some specific embodiments, the DNA construct of the present disclosure comprises:
PsEF1A-6p operably linked to a nuclease and a gRNA;
PsEF1A-7p operably linked to a nuclease and a gRNA;
PsEF1A-0p operably linked to a nuclease and PsUBI3p operably linked to a gRNA;
PsEF1A-1p operably linked to a nuclease and PsUBI3p operably linked to a gRNA;
PsUBI3-SYN3p operably linked to a nuclease and PsEF1A-0p operably linked to a gRNA;
PsUBI3-SYN3p operably linked to a nuclease and PsEF1A-7p operably linked to a gRNA; or
AtUBI11p operably linked to a nuclease, and PsEF1A-1p operably linked to a gRNA.

### 2. Selectable Markers/Reporter Genes

The promoters of the present disclosure may be operably linked to reporter gene or selectable marker gene sequences. The DNA construct of the present disclosure can comprise a nucleic acid sequence encoding a selectable marker operably linked to a promoter molecule. The selectable marker can be operably linked to the promoter of the present disclosure, alone or together with another polynucleotide of interest (e.g., encoding a guide RNA or a nuclease), or any other promoter.

Constructs comprising promoter molecules of the present disclosure operably linked to selectable markers can enable the expression of selectable markers in a ubiquitous constitutive pattern and can enhance transformation rates (i.e., the number of regenerated primary transformants relative to the number of total explants). When a selectable marker and editing reagents are both introduced into a plant or plant part using the promoter molecule of the present disclosure or the DNA construct of the present disclosure, an interplay between selectable marker and editing reagent expression can enable transgene-free segregants to be recovered from the secondary-transformant generation.

Selectable marker genes are utilized for selection of transformed cells or tissues. In accordance with certain embodiments, the polynucleotide sequence of interest can encode a selectable marker or a gene product conferring insecticidal resistance, herbicide tolerance, small RNA expression, nitrogen use efficiency, water use efficiency, or nutritional quality.

Selectable marker genes for selection of transformed cells or tissues can include genes that confer antibiotic resistance or resistance to herbicides. Examples of suitable selectable marker genes include, but are not limited to, genes encoding resistance to chloramphenicol (Herrera Estrella, et al., (1983) EMBO J. 2:987-992); methotrexate (Herrera Estrella, et al., (1983) Nature 303:209-213; Meijer, et al., (1991) Plant Mol. Biol. 16:807-820); hygromycin (Waldron, et al., (1985) Plant Mol. Biol. 5:103-108 and Zhijian, et al., (1995) Plant Science 108:219-227); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-36); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518), herein incorporated by reference in their entirety.

Selectable marker genes include genes encoding antibiotic resistance, such as those encoding neomycin phosphotransferase II (NEO), spectinomycin/streptinomycin resistance (AAD), and hygromycin phosphotransferase (HPT or HGR) as well as genes conferring resistance to herbicidal compounds. Herbicide resistance genes generally code for a modified target protein insensitive to the herbicide or for an enzyme that degrades or detoxifies the herbicide in the plant before it can act. For example, resistance to glyphosate has been obtained by using genes coding for mutant target enzymes, 5-enolpyruvylshikimate-3-phosphate synthase (EPSPS). Genes and mutants for EPSPS are well known, and further described below. Resistance to glufosinate ammonium, bromoxynil, and 2,4-dichlorophenoxyacetate (2,4-D) have been obtained by using bacterial genes encoding PAT or DSM-2, a nitrilase, an AAD-1, or an AAD-12, each of which are examples of proteins that detoxify their respective herbicides.

Herbicides can inhibit the growing point or meristem, including imidazolinone or sulfonylurea, and genes for resistance/tolerance of acetohydroxyacid synthase (AHAS) and acetolactate synthase (ALS) for these herbicides are well known. Glyphosate resistance genes include mutant 5-enolpyruvylshikimate-3-phosphate synthase (EPSPs) and dgt-28 genes (via the introduction of recombinant nucleic acids and/or various forms of in vivo mutagenesis of native EPSPs genes), aroA genes and glyphosate acetyl transferase (GAT) genes, respectively). Resistance genes for other phosphono compounds include bar and pat genes from Streptomyces species, including Streptomyces hygroscopicus and Streptomyces viridichromogenes, and pyridinoxy or phenoxy proprionic acids and cyclohexones (ACCase inhibitor-encoding genes). Exemplary genes conferring resistance to cyclohexanediones and/or aryloxyphenoxypropanoic acid (including haloxyfop, diclofop, fenoxyprop, fluazifop, quizalofop) include genes of acetyl coenzyme A carboxylase (ACCase); Accl-S1, Accl-S2 and Accl-S3. Herbicides can also inhibit photosynthesis, including triazine (psbA and 1s+ genes) or benzonitrile (nitrilase gene). Further, such selectable markers can include positive selection markers such as phosphomannose isomerase (PMI) enzyme.

Selectable marker genes can further include, but are not limited to genes encoding: 2,4-D; neomycin phosphotransferase II; cyanamide hydratase; aspartate kinase; dihydrodipicolinate synthase; tryptophan decarboxylase; dihydrodipicolinate synthase and desensitized aspartate kinase; bar gene; tryptophan decarboxylase; neomycin phosphotransferase (NEO); hygromycin phosphotransferase (HPT or HYG); dihydrofolate reductase (DHFR); phosphinothricin acetyltransferase; 2,2-dichloropropionic acid dehalogenase; acetohydroxyacid synthase; 5-enolpyruvyl-shikimate-phosphate synthase (aroA); haloarylnitrilase; acetyl-coenzyme A carboxylase; dihydropteroate synthase (sul I); and 32 kD photosystem II polypeptide (psbA). Selectable marker genes can further include genes encoding resistance to: chloramphenicol; methotrexate; hygromycin; spectinomycin; bromoxynil; glyphosate; and phosphinothricin.

Other selectable marker genes that could be employed on the expression constructs disclosed herein include, but are not limited to, GUS (beta-glucuronidase; Jefferson, (1987) Plant Mol. Biol. Rep. 5:387), GFP (green fluorescence protein; Chalfie, et al., (1994) Science 263:802), luciferase (Riggs, et al., (1987) Nucleic Acids Res. 15(19):8115 and Luehrsen, et al., (1992) Methods Enzymol. 216:397-414), red fluorescent protein (DsRFP, RFP, etc), beta-galactosidase, and the maize genes encoding for anthocyanin production (Ludwig, et al., (1990) Science 247:449), and the like (See Sambrook, et al., Molecular Cloning: A Laboratory Manual, Third Edition, Cold Spring Harbor Press, N.Y., 2001), herein incorporated by reference in their entirety. The above list of selectable marker genes is not meant to be limiting. Any reporter or selectable marker gene are encompassed by the present disclosure.

### 3. Regulatory RNA/Small RNA

The promoters of the present disclosure may be operably linked to a polynucleotide of interest encoding regulatory RNA or small RNA. The DNA constructs of the present disclosure may comprise a polynucleotide of interest encoding regulatory RNA or small RNA. As used herein, a “regulatory RNA” refers to a non-coding RNA that regulates expression of genes. Regulatory RNAs comprise a heterogeneous group of short and long RNAs, including microRNA (miRNA) and long non-coding RNA (lncRNA). In some embodiments, the regulatory RNA for expression using the spatio-temporal promoter of the present disclosure is one or more of a microRNA (miRNA), a short-hairpin RNA, a guide RNA, a transposase, a homology-directed repair enhancer, and a non-homologous end-joining suppressor.

Various small RNA sequences can be operably linked to the promoters disclosed herein. As used herein, a “small RNA” refers to a polymeric RNA molecule, which is typically non-coding and regulates expression of genes. Types of small RNA can include microRNA (miRNA), piwi-interacting RNA (piRNA), small interfering RNA (siRNA), and small nuclear RNA 9snRNA). Examples of small RNA coding sequences that can be operably linked to the promoters of the present disclosure include delayed fruit ripening/senescence of the anti-efe small RNA delays ripening by suppressing the production of ethylene via silencing of the ACO gene that encodes an ethylene-forming enzyme. The altered lignin production of ccomt small RNA reduces content of guanacyl (G) lignin by inhibition of the endogenous S-adenosyl-L-methionine: trans-caffeoyl CoA 3-O-methyltransferase (CCOMT gene). Further, the black spot bruise tolerance in Solanum verrucosum can be reduced by the Ppo5 small RNA which triggers the degradation of Ppo5 transcripts to block black spot bruise development. Also included is the dvsnf7 small RNA that inhibits Western Corn Rootworm with dsRNA containing a 240 bp fragment of the Western Corn Rootworm Snf7 gene. Modified starch/carbohydrates can result from small RNA such as the pPhL small RNA (degrades PhL transcripts to limit the formation of reducing sugars through starch degradation) and pR1 small RNA (degrades R1 transcripts to limit the formation of reducing sugars through starch degradation). Additionally, benefits such as reduced acrylamide can result from the asnl small RNA that triggers degradation of Asnl to impair asparagine formation and reduce polyacrylamide. Finally, the non-browning phenotype of PGAS PPO suppression small RNA results in suppressing PPO to produce apples with a non-browning phenotype. The above list of small RNAs is not meant to be limiting. Any small RNA encoding sequences are encompassed by the present disclosure.

The regulatory RNA or small RNA can be operably linked to the promoter of the present disclosure, alone or together with another polynucleotide of interest (e.g., encoding a guide RNA or a nuclease), or any other promoter. For example, the regulatory RNA or small RNA can be operably linked to an RNA polymerase III promoter. The regulatory RNA or small RNA can be operably linked to a promoter molecule comprising a nucleic acid sequence that (i) comprises one or more deletions, substitutions, and/or insertions relative to the nucleic acid sequence of any one of SEQ ID NOs: 1-4, 9-39, and 43-46, shares at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%) sequence identity with any one of SEQ ID NOs: 1-4, 9-39, and 43-46, and retains transcription initiation function, (ii) has at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%) sequence identity to any one of SEQ ID NOs: 1-4, 47-49, 51, and 53, and retains transcription initiation function, or (iii) is any one of SEQ ID NOs: 1-4, 47-49, 51, and 53.

### 4. Other Polynucleotides of Interest

Polynucleotides of interest that are suitable for use in the present disclosed constructs include, but are not limited to, polynucleotides of interest that confer resistance to pests or disease, tolerance to herbicides, favorable agronomic traits, such as yield improvement, nitrogen use efficiency, water use efficiency, and nutritional quality.

Various insect resistance genes can be operably linked to the promoters disclosed herein. As examples of insect resistance genes that can be operably linked to the regulatory elements of the subject disclosure, the following traits are provided. Genes that provide exemplary Lepidopteran insect resistance include: cry1A; cry1A.105; cry1Ab; cry1Ab(truncated); cry1Ab Ac (fusion protein); cry1Ac; cry1C; cry1F; cry1Fa2; cry2Ab2; cry2Ae; cry9C; mocrylF; pinII (protease inhibitor protein); vip3A(a); and vip3Aa20. Genes that provide exemplary Coleopteran insect resistance include: cry34Ab1; cry35Ab1; cry3A; cry3Bbl; dvsnf7; and mcry3A. Coding sequences that provide exemplary multi-insect resistance include ecry31.Ab. The above list of insect resistance genes is not meant to be limiting. Any insect resistance genes are encompassed by the present disclosure.

Various herbicide tolerance genes can be operably linked to the promoters disclosed herein. As examples of herbicide tolerance coding sequences that can be operably linked to the regulatory elements of the subject disclosure, the following traits are provided. The glyphosate herbicide contains a mode of action by inhibiting the EPSPS enzyme (5-enolpyruvylshikimate-3-phosphate synthase). This enzyme is involved in the biosynthesis of aromatic amino acids that are essential for growth and development of plants. Various enzymatic mechanisms are known in the art that can be utilized to inhibit this enzyme. The genes that encode such enzymes can be operably linked to any promoters disclosed herein. For example, selectable marker genes include, but are not limited to genes encoding glyphosate resistance genes such as: mutant EPSPS genes including 2mEPSPS genes, cp4 EPSPS genes, mEPSPS genes, dgt-28 genes; aroA genes; and glyphosate degradation genes such as glyphosate acetyl transferase genes (gat) and glyphosate oxidase genes (gox). Resistance genes for glufosinate and/or bialaphos compounds include dsm-2, bar and pat genes. Also included are tolerance genes that provide resistance to 2,4-D such as aad-1 genes (it should be noted that aad-1 genes have further activity on arloxyphenoxypropionate herbicides) and aad-12 genes (it should be noted that aad-12 genes have further activity on pyidyloxyacetate synthetic auxins). Resistance genes for ALS inhibitors (sulfonylureas, imidazolinones, triazolopyrimidines, pyrimidinylthiobenzoates, and sulfonylamino-carbonyl-triazolinones) are known in the art. These resistance genes most commonly result from point mutations to the ALS encoding gene sequence. Other ALS inhibitor resistance genes include hra genes, the csrl-2 genes, Sr-HrA genes, and surB genes. Herbicides that inhibit HPPD include the pyrazolones such as pyrazoxyfen, benzofenap, and topramezone; triketones such as mesotrione, sulcotrione, tembotrione, benzobicyclon; and diketonitriles such as isoxaflutole. These exemplary HPPD herbicides can be tolerated by known traits. Examples of HPPD inhibitors include hppdPF W336 genes (for resistance to isoxaflutole) and avhppd-03 genes (for resistance to meostrione). An example of oxynil herbicide tolerant traits include the bxn gene, which has been showed to impart resistance to the herbicide/antibiotic bromoxynil. Resistance genes for dicamba include the dicamba monooxygenase gene (dmo) as disclosed in International PCT Publication No. WO 2008/105890. Resistance genes for PPO or PROTOX inhibitor type herbicides (e.g., acifluorfen, butafenacil, flupropazil, pentoxazone, carfentrazone, fluazolate, pyraflufen, aclonifen, azafenidin, flumioxazin, flumiclorac, bifenox, oxyfluorfen, lactofen, fomesafen, fluoroglycofen, and sulfentrazone) are known in the art. Exemplary genes conferring resistance to PPO include over expression of a wild-type Arabidopsis thaliana PPO enzyme (Lermontova I and Grimm B, (2000) Overexpression of plastidic protoporphyrinogen IX oxidase leads to resistance to the diphenyl-ether herbicide acifluorfen. Plant Physiol 122:75-83.), the B. subtilis PPO gene (Li, X. and Nicholl D. 2005. Development of PPO inhibitor-resistant cultures and crops. Pest Manag. Sci. 61:277-285 and Choi K W, Han O, Lee H J, Yun Y C, Moon Y H, Kim M K, Kuk Y I, Han S U and Guh J O, (1998) Generation of resistance to the diphenyl ether herbicide, oxyfluorfen, via expression of the Bacillus subtilis protoporphyrinogen oxidase gene in transgenic tobacco plants. Biosci Biotechnol Biochem 62:558-560.) Resistance genes for pyri di noxy or phenoxy proprionic acids and cyclohexones include the ACCase inhibitor-encoding genes (e.g., Accl-S1, Accl-S2 and Accl-S3). Exemplary genes conferring resistance to cyclohexanedi ones and/or aryloxyphenoxypropanoic acid include haloxyfop, diclofop, fenoxyprop, fluazifop, and quizalofop. Finally, herbicides can inhibit photosynthesis, including triazine or benzonitrile are provided tolerance by psbA genes (tolerance to triazine), 1s+ genes (tolerance to triazine), and nitrilase genes (tolerance to benzonitrile). The above list of herbicide tolerance genes is not meant to be limiting. Any herbicide tolerance genes are encompassed by the present disclosure.

Various agronomic trait genes can be operably linked to the promoters disclosed herein. As examples of agronomic trait coding sequences that can be operably linked to the regulatory elements of the subject disclosure, the following traits are provided. Delayed fruit softening as provided by the pg genes inhibit the production of polygalacturonase enzyme responsible for the breakdown of pectin molecules in the cell wall, and thus causes delayed softening of the fruit. Further, delayed fruit ripening/senescence of acc genes act to suppress the normal expression of the native acc synthase gene, resulting in reduced ethylene production and delayed fruit ripening. Whereas, the accd genes metabolize the precursor of the fruit ripening hormone ethylene, resulting in delayed fruit ripening. Alternatively, the sam-k genes cause delayed ripening by reducing S-adenosylmethionine (SAM), a substrate for ethylene production. Drought stress tolerance phenotypes as provided by cspB genes maintain normal cellular functions under water stress conditions by preserving RNA stability and translation. Another example includes the EcBetA genes that catalyze the production of the osmoprotectant compound glycine betaine conferring tolerance to water stress. In addition, the RmBetA genes catalyze the production of the osmoprotectant compound glycine betaine conferring tolerance to water stress. Photosynthesis and yield enhancement is provided with the bbx32 gene that expresses a protein that interacts with one or more endogenous transcription factors to regulate the plant's day/night physiological processes. Ethanol production can be increase by expression of the amy797E genes that encode a thermostable alpha-amylase enzyme that enhances bioethanol production by increasing the thermostability of amylase used in degrading starch. Finally, modified amino acid compositions can result by the expression of the cordapA genes that encode a dihydrodipicolinate synthase enzyme that increases the production of amino acid lysine. The above list of agronomic trait coding sequences is not meant to be limiting. Any agronomic trait coding sequence is encompassed by the present disclosure.

The polynucleotides of interest can be synthesized for optimal expression in a plant. For example, a polynucleotide of interest can have been modified by codon optimization to enhance expression in plants. An insecticidal resistance transgene, an herbicide tolerance transgene, a nitrogen use efficiency transgene, a water use efficiency transgene, a nutritional quality transgene, a DNA binding transgene, or a selectable marker transgene/heterologous coding sequence can be optimized for expression in a particular plant species or alternatively can be modified for optimal expression in dicotyledonous or monocotyledonous plants. Plant preferred codons may be determined from the codons of highest frequency in the proteins expressed in the largest amount in the particular plant species of interest. For example, a polynucleotide of interest, e.g., a coding sequence, gene, heterologous coding sequence, or transgene/heterologous coding sequence can be designed to be expressed in plants at a higher level resulting in higher transformation efficiency. Guidance regarding the optimization and production of synthetic DNA sequences can be found in, for example, WO2013016546, WO2011146524, WO1997013402, U.S. Pat. Nos. 6,166,302, and 5,380,831, herein incorporated by reference.

### B. Other Elements of Constructs

As disclosed herein, the DNA constructs of present disclosure can comprise a promoter molecule (e.g., a PsEF1Ap, a homolog, a fragment, or a variant thereof, e.g., with one or more deletions, substitutions, and/or insertions) operably linked to a polynucleotide of interest. In addition, the DNA constructs can comprise one or more of the following elements, and can also comprise other elements not exemplified herein.

### 1. Transfer DNA

The recombinant DNA constructs of the present disclosure may contain T-DNA sequences. For example, a recombinant DNA construct of the present disclosure may contain T-DNA of tumor-inducing (Ti) plasmid of Agrobacterium tumefaciens. Alternatively, a recombinant DNA construct of the present disclosure may contain T-DNA of tumor-inducing (Ti) plasmid of Agrobacterium rhizogenes. The vir genes of the Ti plasmid may help in transfer of T-DNA of a recombinant DNA construct into nuclear DNA genome of a host plant. For example, Ti plasmid of Agrobacterium tumefaciens may help in transfer of T-DNA of a recombinant DNA construct of the present disclosure into nuclear DNA genome of a host plant, thus enabling the transfer of a guide RNA of the present disclosure into nuclear DNA genome of a host plant (e.g., a pea plant).

### 2. Regulatory Signals

In some embodiments, a recombinant DNA construct described herein may contain additional regulatory signals, including, but not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

### 3. Terminators

A transcription terminator may also be included in the expression cassettes of DNA constructs of the present invention. Plant terminators are known in the art and include those available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

### C. Vectors and Cells Comprising the Promoter or the Construct

In some aspects, further disclosed herein are vectors containing promoter molecules or DNA constructs of the present disclosure. As used herein, “vector” refers to a nucleotide molecule (e.g., a plasmid, cosmid), bacterial phage, or virus for introducing a nucleotide construct, for example, a recombinant DNA construct, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance. In some specific embodiments, provided herein are expression cassettes located on a vector comprising the promoter molecule of the present disclosure operably linked to a polynucleotide of interest (e.g., encoding a nuclease and/or a guide RNA).

In some embodiments, a vector is a plasmid containing a recombinant DNA construct of the present disclosure. In some embodiments, a vector is a cosmid containing a DNA construct of the present disclosure.

In some embodiments, a vector is a recombinant virus containing a recombinant DNA construct of the present disclosure. A recombinant virus described herein can be a recombinant lentivirus, a recombinant retrovirus, a recombinant cucumber mosaic virus (CMV), a recombinant tobacco mosaic virus (TMV), a recombinant cauliflower mosaic virus (CaMV), a recombinant odontoglossum ringspot virus (ORSV), a recombinant tomato mosaic virus (ToMV), a recombinant bamboo mosaic virus (BaMV), a recombinant cowpea mosaic virus (CPMV), a recombinant potato virus X (PVX), a recombinant Bean yellow dwarf virus (BeYDV), or a recombinant turnip vein-clearing virus (TVCV).

In some aspects, provided herein are cells comprising a nucleic acid molecule (comprising a promoter sequence) of the present disclosure, a DNA construct of the present disclosure, or a vector of the present disclosure. In some embodiments, the cell is selected from the group consisting of a plant cell, a bacterial cell, and a fungal cell. For example, the present disclosure provides a bacterium, e.g., an Agrobacterium tumefaciens, containing a promoter molecule of the present disclosure or a DNA construct of the present disclosure for expressing a polynucleotide of interest, e.g., editing reagents for genomic loci of interest, selectable marker, or a regulatory RNA. The cells of the present disclosure may be grown, or have been grown, in a cell culture.

### V. Plants Comprising a Heterologous Promoter and a Polynucleotide of Interest

Disclosed herein are plants, plant parts (e.g., juice, pulp, seed, grain, fruit, flowers, nectar, embryos, pollen, ovules, leaves, stems, branches, bark, kernels, ears, cobs, husks, stalks, roots, root tips, anthers, etc.), or plant products (e.g., plant extract, plant concentrate, plant powder, plant protein, and plant biomass) comprising the nucleic acid molecule (comprising the promoter sequence), the DNA construct, or the cell of the present disclosure. Also disclosed herein plants, plant parts (e.g., juice, pulp, seed, grain, fruit, flowers, nectar, embryos, pollen, ovules, leaves, stems, branches, bark, kernels, ears, cobs, husks, stalks, roots, root tips, anthers, etc.), or plant products (e.g., plant extract, plant concentrate, plant powder, plant protein, and plant biomass) generated by introducing the nucleic acid molecule (comprising the promoter sequence), the DNA construct, or the cell of the present disclosure into the plants or plant parts.

In some aspects, the present disclosure provides plants or plant parts comprising a promoter molecule provided herein, or a DNA construct provided herein comprising, in operable linkage, a promoter molecule provided herein and a polynucleotide of interest. The DNA construct can include one promoter molecule operably linked to one or more polynucleotides of interest. Alternatively, the DNA construct can include more than one promoter molecules each operably linked to one or more polynucleotides of interest, wherein at least one of the promoter molecules are a promoter molecule of the present disclosure (e.g., a PsEF1A promoter, a homolog, a fragment, or a variant thereof).

The promoter molecule in the plant or plant part can comprise a nucleic acid sequence that shares at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with a nucleic acid sequence of a PsEF1A promoter or a homolog thereof, and retains transcription initiation function. The promoter molecule can comprise a nucleic acid sequence of a PsEF1A promoter or a homolog thereof. The PsEF1A promoter homologs can be PsEF1Ap homologs (e.g., PsEF1A-0p, PsEF1A-1p PsEF1A-6p PsEF1A-7p), AhEF1Ap homologs, CaEF1Ap homologs, LaEF1Ap homologs, LjEF1Ap homologs, MtEF1Ap homologs, PvEF1A-4Ap, PvEF1A-4Bp, PvEF1Ap homologs, TpEF1Ap homologs, and VuEF1Ap homologs, e.g., any one of SEQ ID NOs: 1-4 and 9-46. The promoter molecule of the plant or plant part can comprise a nucleic acid sequence that shares at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with a nucleic acid sequence of any one of SEQ ID NOs: 1-4, 9-39, and 43-46, and retains transcription initiation function. The promoter molecule can comprise a nucleic acid sequence of any one of SEQ ID NOs: 1-4, 9-39, and 43-46.

In some embodiments, the promoter molecule of the DNA construct can comprise the nucleic acid sequence of PsEF1A-0p, PsEF1A-1p, PsEF1A-6p, PsEF1A-7p, which are set forth as SEQ ID NOs: 1-4, respectively. The promoter sequence can share at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-4, and retain transcription initiation function.

The promoter molecule in the plant or plant part can also comprise a promoter sequence having one or more deletions, substitutions, and/or insertions (e.g., synthetic deletions, substitutions, and/or insertions) relative to the nucleic acid sequence of a PsEF1A promoter or a homolog thereof (which can include a 5′UTR, a 5′UTR intron, an exon sequence from a coding region, and/or an intron sequence from a coding region). Said one or more deletions, substitutions, and/or insertions (e.g., synthetic deletions and/or substitutions) can be directed to structurally-repetitive sequences such as a microsatellite sequence, a sequence duplication, a palindrome, a homo-polymetric tract, to enhance the promoter stability, function, synthesis, or use thereof, and/or to enhance the expression of an operably linked polynucleotide. The one or more deletions, substitutions, and/or insertions (e.g., synthetic deletions, substitutions, and/or insertions) can be made in a 3′ region of said promoter sequence and/or a 5′UTR, a 5′UTR intron, an exon sequence from a coding region, and/or an intron sequence from a coding region of the promoter sequence. The PsEF1A promoter homologs can be PsEF1Ap homologs (e.g., PsEF1A-0p, PsEF1A-1p PsEF1A-6p PsEF1A-7p), AhEF1Ap homologs, CaEF1Ap homologs, LaEF1Ap homologs, LjEF1Ap homologs, MtEF1Ap homologs, PvEF1A-4Ap, PvEF1A-4Bp, PvEF1Ap homologs, TpEF1Ap homologs, and VuEF1Ap homologs, e.g., any one of SEQ ID NOs: 1-4 and 9-46. The promoter sequence can share at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-4 and 9-39, and 43-46, and retain transcription initiation function. In some embodiments, the promoter sequence (which can include a 5′UTR, a 5′UTR intron, an exon sequence from a coding region, and/or an intron sequence from a coding region) comprises one or more deletions, substitutions, and/or insertions (e.g., synthetic deletions, substitutions, and/or insertions) relative to the nucleic acid sequence of P sEF 1 A-Op, PsEF IA-1p, PsEF IA-6p, PsEF IA-7p, which are set forth as SEQ ID NOs: 1-4, respectively. The promoter sequence can share at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-4, and retain transcription initiation function. For example, the promoter sequence can share at least 80% sequence identity with any one of SEQ ID NOs: 5-8, or comprise the nucleic acid sequence of any one of SEQ ID NOs: 5-8, and retain transcription initiation function.

The coding sequence(s) of the DNA construct can encode any polynucleotide of interest, for expression driven by the promoter molecule of the present disclosure. For example, the coding sequence(s) may encode editing reagents (e.g., a guide RNA, a nuclease) targeting any gene or genomic site of interest, regulatory RNA, a selectable marker/reporter, an enzyme, a transcription factor, a receptor, a ligand, or a molecule that confers resistance to pests or disease, tolerance to herbicides, and/or advantageous agronomic traits (e.g., yield improvement, nitrogen use efficiency, water use efficiency, and nutritional quality).

The promoters in the plants or plant parts may be operably linked to nuclease sequences. The DNA constructs in the plants or plant parts may comprise nuclease sequences. The nuclease can be a clustered, regularly interspaced, short palindromic repeats (CRISPR)-associated Cas endonuclease. In specific embodiments, the CRISPR nuclease is a Cas12a nuclease, i.e., a Cpf1 nuclease. In a specific embodiment, the Cas12a nuclease is a McCpf1 nuclease, e.g., a Mc.2Cpf1 2C-NLS (nuclear localization signal) nuclease. In some embodiments, the CRISPR nuclease shares at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with SEQ ID NO: 62 and retains nuclease activity, or comprises the sequence of SEQ ID NO: 62. In some embodiments, the nuclease is further operably linked to one or more nuclear localization sequences (NLSs) and/or one or more epitope tags. Any NLS or epitope tag can be linked to the nuclease provided herein. For example, the nuclease can be linked to a SV40-nucleophosmin 2 (SV40-NPM2) sequence. In some embodiments, the CRISPR nuclease with NLS shares at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with SEQ ID NO: 63 and retains the nuclease activity and the NLS activity, or comprises the sequence of SEQ ID NO:

63.

The promoters in the plants or plant parts may be operably linked to coding sequences for guide RNAs. The DNA constructs in the plants or plant parts may comprise coding sequences for guide RNAs.

In some embodiments, the polynucleotide of interest operably linked to the promoter molecule described herein encodes a guide RNA or a nuclease. In some embodiments, the plant or plant part comprises a DNA construct comprising one or more polynucleotides of interest encoding a guide RNA and a nuclease, both operably linked to the same promoter molecule of the DNA construct. In some embodiments, polynucleotides encoding a guide RNA and a nuclease can be each operably linked to different promoter molecules, at least one of which is a promoter molecule of the present disclosure (e.g., a PsEF1Ap, a homolog, a fragment, or a derivative thereof e.g., with one or more deletions, substitutions, and/or insertions).

In some embodiments, the polynucleotide of interest of the DNA construct operably linked to the promoter molecule encodes a guide RNA, and the plant or plant part further comprises, in operable linkage, a nucleic acid molecule encoding a second promoter molecule and a polynucleotide encoding a nuclease. In some embodiments, the second promoter molecule is an RNA polymerase II promoter molecule. The second promoter molecule operably linked to a nuclease can comprise a nucleic acid sequence that (i) comprises one or more deletions, substitutions, and/or insertions relative to the nucleic acid sequence of any one of SEQ ID NOs: 1-4, 9-39, and 43-46, shares at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%) sequence identity with any one of SEQ ID NOs: 1-4, 9-39, and 43-46, and retains transcription initiation function, (ii) has at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%) sequence identity to any one of SEQ ID NOs: 1-4, 9-39, 43-52, and 56-61, and retains transcription initiation function, or (iii) is any one of SEQ ID NOs: 1-4, 9-39, 43-52, and 56-61.

In some embodiments, the polynucleotide of interest of the DNA construct operably linked to the promoter molecule encodes a nuclease, and the plant or plant part further comprises, in operable linkage: a nucleic acid molecule encoding a second promoter molecule and a polynucleotide encoding a guide RNA. In some embodiments, the second promoter molecule is an RNA polymerase III (pol III) promoter molecule. The second promoter molecule operably linked to a guide RNA can comprise a nucleic acid sequence that (i) comprises one or more deletions, substitutions, and/or insertions relative to the nucleic acid sequence of any one of SEQ ID NOs: 1-4, 9-39, and 43-46, shares at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%) sequence identity with any one of SEQ ID NOs: 1-4, 9-39, and 43-46, and retains transcription initiation function, (ii) has at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%) sequence identity to any one of SEQ ID NOs: 1-4, 47-49, 51, and 53, and retains transcription initiation function, or (iii) is any one of SEQ ID NOs: 1-4, 47-49, 51, and 53.

Where the plant or plant part comprises one or more DNA constructs comprising coding sequences of a nuclease and a guide RNA, exemplary combinations of promoters for a nuclease and a guide RNA coding sequences are indicated with “X” in Table 1. Each promoter in Table 1 is meant to include its variants and fragments, e.g., promoter molecules that share at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence similarity with the promoter molecule indicated, including promoter molecules comprising one or more deletions, substitutions, and/or insertions (e.g., synthetic deletions, substitutions, and/or insertions) thereof and retaining transcription initiation function. Combinations are not limited to those listed herein. In some specific embodiments, the plant or plant part of the present disclosure comprises:
PsEF1A-6p operably linked to a polynucleotide encoding a nuclease and a gRNA;
PsEF1A-7p operably linked to a polynucleotide encoding a nuclease and a gRNA;
PsEF1A-Op operably linked to a polynucleotide encoding a nuclease and PsUBI3p operably linked to a polynucleotide encoding a gRNA;
PsEF1A-1p operably linked to a polynucleotide encoding a nuclease and PsUBI3p operably linked to a polynucleotide encoding a gRNA;
PsUBI3-SYN3p operably linked to a polynucleotide encoding a nuclease and PsEF1A-0p operably linked to a polynucleotide encoding a gRNA;
PsUBI3-SYN3p operably linked to a polynucleotide encoding a nuclease and PsEF1A-7p operably linked to a polynucleotide encoding a gRNA; or
AtUBI11p operably linked to a polynucleotide encoding a nuclease, and PsEF1A-1p operably linked to a polynucleotide encoding a gRNA.

In some embodiments, the plant or plant part of the present disclosure can comprise a polynucleotide encoding a selectable marker, a regulatory RNA, and/or a small RNA. The selectable marker, the regulatory RNA, and/or the small RNA can be operably linked to the promoter of the present disclosure, alone or together with another polynucleotide of interest (e.g., encoding a guide RNA or a nuclease), or any other promoter. For example, the regulatory RNA or small RNA can be operably linked to an RNA polymerase III promoter.

Additionally or alternatively, the regulatory RNA or small RNA can be operably linked to a promoter molecule comprising a nucleic acid sequence that (i) comprises one or more deletions, substitutions, and/or insertions relative to the nucleic acid sequence of any one of SEQ ID NOs: 1-4, 9-39, and 43-46, shares at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%) sequence identity with any one of SEQ ID NOs: 1-4, 9-39, and 43-46, and retains transcription initiation function, (ii) has at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%) sequence identity to any one of SEQ ID NOs: 1-4, 47-49, 51, and 53, and retains transcription initiation function, or (iii) is any one of SEQ ID NOs: 1-4, 47-49, 51, and 53.

A plant or plant part of the present disclosure can be a monocot. Alternatively, a plant or plant part of the present disclosure can be a dicot. A plant or plant part of the present disclosure can be a crop plant or part of a crop plant. Examples of crop plants include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. Additionally, or alternatively, a plant or plant part of the present disclosure can be a legume, i.e., a plant belonging to the family Fabaceae (or Leguminosae), or a part (e.g., fruit or seed) of such a plant. When used as a dry grain, the seed of a legume is also called a pulse. Examples of legume include, without limitation, beans (Phaseolus spp.), soybean (Glycine max), pea (Pisum sativum), chickpea (Cicer arietinum), cowpea (Vigna unguiculata), peanut (Arachis hypogaea), lentils (Lens culinaris, Lens esculenta), fava bean (Viciafaba), mung bean (Vigna radiata), lupins (Lupinus spp.), e.g., white lupin (Lupinus albus), mesquite (Prosopis spp.), carob (Ceratonia siliqua), tamarind (Tamarindus indica), alfalfa (Medicago sativa), birdsfoot trefoil (Lotus japonicus), and clover (Trifohum spp.), e.g., red clover (Trifohum pratense). Additionally, or alternatively, a plant or plant part of the present disclosure can be an oilseed plant (e.g., canola (Brassica napus), cotton (Gossypium sp.), camelina (Camelina sativa) and sunflower (Hehanthus sp.)), or other species including wheat (Triticum sp., such as Triticum aestivum L. ssp. Aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), hemp (Cannabis sativa). For example, a plant or plant part of the present disclosure can be Pisum sativum or a part of Pisum sativum.

Also provided herein are plant parts (e.g., juice, pulp, seed, grain, fruit, flowers, nectar, embryos, pollen, ovules, leaves, stems, branches, bark, kernels, ears, cobs, husks, stalks, roots, root tips, anthers, etc.), plant extract (e.g., protein, sweetener, antioxidants, alkaloids, etc.), plant concentrate (e.g., whole plant concentrate, plant part concentrate, or protein concentrate), plant powder [e.g., formulated powder, such as formulated plant part powder (e.g., seed flour)], and plant biomass (e.g., dried biomass, such as crushed and/or powdered biomass) obtained from plants of the present disclosure. Also provided herein are seeds, such as a representative sample of seeds, from a plant of the present disclosure.

Also provided herein are food and/or beverage products containing plant compositions (e.g., plant parts, plant extract, plant concentrate, plant powder, plant protein, and plant biomass) described hereinabove, such as plant compositions derived from the plants or plant parts of the present disclosure. Such food and/or beverage products include, without limitation, shakes, juices, health drinks, alternative meat products (e.g., meatless burger patties, meatless sausages, etc.), alternative egg products (e.g., eggless mayo), and non-dairy products (e.g., non-dairy whipped toppings, non-dairy milk, non-dairy creamer, non-dairy milk shakes, etc, and condiments. A food and/or beverage product that contains plant compositions obtained from plants or plant parts of the present disclosure can have desired traits, compared to a similar or comparable food and/or beverage product that contains plant compositions obtained from a control plant or plant part.

### VI. Methods of Expressing a Polynucleotide of Interest in a Plant

Disclosed herein are methods of expressing a nucleotide sequence of interest in a plant or plant part (e.g., juice, pulp, seed, fruit, flower, nectar, embryo, pollen, ovule, leaf, stem, branch, bark, kernel, ear, cob, husk, stalk, root, root tip, anther) by introducing into the plant or the plant part the promoter molecule or the DNA construct of the present disclosure. Also disclosed herein are methods of transforming a plant or plant part by introducing into the plant or the plant part the promoter molecule or the DNA construct of the present disclosure and regenerating a transformed plant or plant part from said plant cell. In some embodiments, the promoter molecule or the DNA construct is introduced into the plant or the plant part by stable transformation. In other embodiments, the promoter molecule or the DNA construct is introduced into the plant by transient transformation.

### A. Transformation of Plants

Provided herein are methods for transforming plants or plant parts by introducing into the plants or plant parts a construct for expressing a polynucleotide of interest, or for introducing one or more mutations (e.g., insertions, substitutions, or deletions) at a desired target site in the plant genome. The term “transform” or “transformation” refers to any method used to introduce polypeptides or polynucleotides into plant cells. For purpose of the present disclosure, the transformation can be: “stable transformation”, wherein the transformation construct (e.g., a construct comprising sequences encoding guide RNA and/or a nuclease for use in the methods of the present invention) is introduced into a host (e.g., a host plant, plant part, plant cell, etc.) and integrates into the genome of the host and is capable of being inherited by the progeny thereof; or “transient transformation”, wherein the transformation construct (e.g., a construct comprising a guide RNA and/or a polynucleotide encoding a nuclease for use in the methods of the present invention) is introduced into a host (e.g., a host plant, plant part, plant cell, etc.) and expressed temporarily. The methods disclosed herein can also be used for introduction of polynucleotides of interest or mutations at target sites of interest in a plant genome and/or modification of native plant gene expression to achieve desirable plant traits.

Sequences encoding any polynucleotide of interest operably linked to a promoter disclosed herein can be introduced into a plant cell, organelle, or plant embryo by a variety of means of transformation, including microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration [see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lecl transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D′Halluin et al. (1992)Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens)]; all of which are herein incorporated by reference.

Agrobacterium-and biolistic-mediated transformation remain the two predominantly employed approaches. However, transformation may be performed by infection, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, Agrobacterium and viral mediated (Caulimoriviruses, Geminiviruses, RNA plant viruses), liposome mediated and the like. Methods disclosed herein are not limited to any size of nucleic acid sequences that are introduced, and thus one could introduce a nucleic acid comprising a single nucleotide (e.g. an insertion) into a nucleic acid of the plant and still be within the teachings described herein. Nucleic acids introduced in substantially any useful form, for example, on supernumerary chromosomes (e.g. B chromosomes), plasmids, vector constructs, additional genomic chromosomes (e.g. substitution lines), and other forms is also anticipated. It is envisioned that new methods of introducing nucleic acids into plants and new forms or structures of nucleic acids will be discovered and yet fall within the scope of the claimed invention when used with the teachings described herein.

More than one polynucleotides of interest [e.g., encoding editing reagents (e.g., a guide RNA, a nuclease) targeting any gene or genomic site of interest, a regulatory RNA, a small RNA, a selectable marker/reporter, an enzyme, a transcription factor, a receptor, a ligand, a molecule that confers resistance to pests or disease, tolerance to herbicides, and/or advantageous agronomic traits (e.g., yield improvement, nitrogen use efficiency, water use efficiency, and nutritional quality)] can be introduced into the plant, plant cell, plant organelle, or plant embryo simultaneously or sequentially. More than one polynucleotides of interest can be introduced into the plant, plant cell, plant organelle, or plant embryo by introducing one DNA construct that comprise all the polynucleotides of interest operably linked to one or more promoters. Alternatively, more than one polynucleotides of interest can be introduced into the plant, plant cell, plant organelle, or plant embryo by introducing more than one DNA constructs that each comprise some of the polynucleotides of interest operably linked to one or more promoters simultaneously or sequentially. For example, a selectable marker and editing reagents can be introduced into the plant, plant cell, plant organelle, or plant embryo in one DNA construct, or in more than one DNA construct simultaneously or sequentially. The amount or ratio of more than one polynucleotides of interest, or molecules encoded therein, can be adjusted by adjusting the amount or concentration of the polynucleotides and/or timing and dosage of introducing the polynucleotides into the plant or plant part. In specific embodiments, polynucleotides of interest encode nuclease and guide RNA(s), and the ratio of the nuclease (or encoding nucleic acid) to the guide RNA(s) (or encoding DNA) generally will be about stoichiometric such that the two components can form an RNA-protein complex with the target DNA. In one embodiment, DNA encoding a nuclease and DNA encoding a guide RNA are delivered together within the plasmid vector.

The cells that have been transformed may be cultured and grown into plants in accordance with conventional ways. See, for example, McCormick et al. (1986) Plant Cell Reports 5:81-84. In this manner, the present invention provides transformed plants or plant parts, transformed seed (also referred to as “transgenic seed”) or transformed plant progenies having a nucleic acid modification stably incorporated into their genome.

The present invention may be used for transformation of any plant species, e.g., both monocots and dicots. The present invention can be used for transformation of crop plants or part of crop plants, e.g., corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana tabacum), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, and conifers. Additionally or alternatively, the present invention can be used for transformation of a legume, i.e., a plant belonging to the family Fabaceae (or Leguminosae), or a part (e.g., fruit or seed) of such a plant, e.g., beans (Phaseolus spp.), soybean (Glycine max), pea (Pisum sativum), chickpea (Cicer arietinum), cowpea (Vigna unguiculata), peanut (Arachis hypogaea), lentils (Lens culinaris, Lens esculenta), fava bean (Viciafaba), mung bean (Vigna radiata), lupins (Lupinus spp.), e.g., white lupin (Lupinus albus), mesquite (Prosopis spp.), carob (Ceratonia siliqua), tamarind (Tamarindus indica), alfalfa (Medicago sativa), birdsfoot trefoil (Lotus japonicus), and clover (Trifolium spp.), e.g., red clover (Trifolium pratense). Additionally or alternatively, the present invention can be used for transformation of an oilseed plant (e.g., canola (Brassica napus), cotton (Gossypium sp.), camelina (Camelina sativa) and sunflower (Helianthus sp.)), or other species including wheat (Triticum sp., such as Triticum aestivum L. ssp. aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), hemp (Cannabis sativa). In specific embodiments, the present invention can be used for transformation of Pisum sativum or a part of Pisum sativum.

Also provided herein are plants and plant parts generated by the methods of the present disclosure, and plant parts (e.g., juice, pulp, seed, fruit, flowers, nectar, embryos, pollen, ovules, leaves, stems, branches, bark, kernels, ears, cobs, husks, stalks, roots, root tips, anthers, etc.), plant extract (e.g., sweetener, antioxidants, alkaloids, etc.), plant concentrate (e.g., whole plant concentrate or plant part concentrate), plant powder [e.g., formulated powder, such as formulated plant part powder (e.g., seed flour)], and plant biomass (e.g., dried biomass, such as crushed and/or powdered biomass), and food or beverage products obtained from plants of the present disclosure. Also provided herein are seeds, such as a representative sample of seeds, from a plant generated by the methods of the present disclosure.

Molecules encoded by the DNA constructs of the present disclosure (e.g., promoters, editing reagents) may be found in plants or plant parts to which the DNA constructs have been introduced, or plants or plant parts regenerated therefrom according to the methods of the present disclosure. Mutations introduced by the methods using the DNA constructs encoding editing reagents may be found in plants or plant parts to which the DNA constructs have been introduced, or plants or plant parts regenerated therefrom according to the methods of the present disclosure. Mutations can also be found in plant parts, plant extract, plant concentrate, plant powder, and plant biomass obtained from such plants.

Also provided herein are food and/or beverage products containing plant compositions (e.g., plant parts, plant extract, plant concentrate, plant powder, plant protein, and plant biomass) described hereinabove, such as plant compositions derived from the plants or plant parts of the present disclosure. Such food and/or beverage products include, without limitation, shakes, juices, health drinks, alternative meat products (e.g., meatless burger patties, meatless sausages, etc.), alternative egg products (e.g., eggless mayo), and non-dairy products (e.g., non-dairy whipped toppings, non-dairy milk, non-dairy creamer, non-dairy milk shakes, etc, and condiments. A food and/or beverage product that contains plant compositions obtained from plants or plant parts of the present disclosure can have desired traits, compared to a similar or comparable food and/or beverage product that contains plant compositions obtained from a control plant or plant part.

While the invention is described in terms of transformed plants, it is recognized that transformed organisms of the invention also include plant cells, plant protoplasts, plant cell tissue cultures from which plants can be regenerated, plant calli, plant clumps, and plant cells that are intact in plants or parts of plants such as embryos, pollen, ovules, seeds, grains, leaves, flowers, branches, fruit, kernels, ears, cobs, husks, stalks, roots, root tips, anthers, and the like. Progeny, variants, and mutants of the regenerated plants are also included within the scope of the invention, provided that these parts comprise the introduced polynucleotides.

### B. Expressing a Polynucleotide of Interest in a Plant

In some aspects, the present disclosure provides methods of expressing a polynucleotide of interest in a plant or plant part comprising introducing a DNA construct into the plant or plant part, wherein the DNA construct comprises, in operable linkage, a nucleic acid molecule comprising a promoter sequence and a polynucleotide of interest. In some aspects, provided herein is a method of transforming a plant or plant part by introducing a DNA construct, comprising a promoter molecule operably linked to a polynucleotide of interest into a plant cell, and regenerating a transformed plant or plant part from said plant cell. The DNA constructs of the methods may each comprise one promoter operably linked to one polynucleotide of interest. The DNA constructs of the methods may each comprise more than one polynucleotides of interest, or a polynucleotide encoding more than one molecules of interest, that are operably linked to the promoter of the present disclosure (e.g., a PsEF1A promoter, a homolog, a fragment, or a variant thereof). The DNA constructs may comprise one promoter of the present disclosure (e.g., a PsEF1A promoter, a homolog, a fragment, or a variant thereof) and operably linked to one, or more than one, polynucleotides of interest. The DNA constructs may comprise more than one promoter molecules, at least one of which is a promoter of the present disclosure (e.g., a PsEF1A promoter, a homolog, a fragment, or a variant thereof), each promoter operably linked to one, or more than one, polynucleotides of interest. The DNA constructs may comprise more than one promoters of the present disclosure (e.g., a PsEF1A promoter, a homolog, a fragment, or a variant thereof), each of which operably linked to one, or more than one, polynucleotides of interest. The polynucleotides or the molecules of interest can have similar types of functions (e.g., more than one editing reagents, or polynucleotides encoding them) or different types of functions (e.g., a selectable marker, a nuclease, and a guide RNA, or polynucleotides encoding them).

The method of the present disclosure can comprise introducing into a plant, plant part, or plant cell one promoter molecule operably linked to one polynucleotide of interest. The method can also comprise introducing into a plant, plant part, or plant cell more than one polynucleotides of interest simultaneously or sequentially. More than one polynucleotides of interest can be introduced into the plant, plant part, or plant cell by introducing one DNA construct that comprise all the polynucleotides of interest operably linked to one or more promoters. Alternatively, more than one polynucleotides of interest can be introduced into the plant, plant part, or plant cell by introducing more than one DNA constructs that each comprise some of the polynucleotides of interest operably linked to one or more promoters simultaneously or sequentially.

The promoter molecule according to the methods of the present disclosure can comprise a nucleic acid sequence that shares at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with a nucleic acid sequence of a PsEF1A promoter or a homolog thereof, and retains transcription initiation function. The promoter molecule can comprise a nucleic acid sequence of a PsEF1A promoter or a homolog thereof. The PsEF1A promoter homologs can be PsEF1Ap homologs (e.g., PsEF1A-0p, PsEF1A-1p PsEF1A-6p PsEF1A-7p), AhEF1Ap homologs, CaEF1Ap homologs, LaEF1Ap homologs, LjEF1Ap homologs, MtEF1Ap homologs, PvEF1A-4Ap, PvEF1A-4Bp, PvEF1Ap homologs, TpEF1Ap homologs, and VuEF1Ap homologs, e.g., any one of SEQ ID NOs: 1-4 and 9-46. The promoter molecules according to the methods can comprise a nucleic acid sequence that shares at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with a nucleic acid sequence of any one of SEQ ID NOs: 1-4, 9-39, and 43-46, and retains transcription initiation function. The promoter molecules can comprise a nucleic acid sequence of any one of SEQ ID NOs: 1-4, 9-39, and 43-46.

In some embodiments, the promoter molecule according to the methods described herein can comprise the nucleic acid sequence of PsEF1A-0p, PsEF1A-1p, PsEF1A-6p, PsEF1A-7p, which are set forth as SEQ ID NOs: 1-4, respectively. The promoter sequence can share at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-4, and retain transcription initiation function.

The promoter molecule according to the methods described herein can also comprise a promoter sequence having one or more deletions, substitutions, and/or insertions (e.g., synthetic deletions, substitutions, and/or insertions) relative to the nucleic acid sequence of a PsEF1A promoter or a homolog thereof (which can include a 5′UTR, a 5′UTR intron, an exon sequence from a coding region, and/or an intron sequence from a coding region). The one or more deletions, substitutions, and/or insertions (e.g., synthetic deletions and/or substitutions) can be directed to structurally-repetitive sequences such as a microsatellite sequence, a sequence duplication, a palindrome, a homo-polymetric tract, to enhance the promoter stability, function, synthesis, or use thereof, and/or to enhance the expression of an operably linked polynucleotide. The one or more deletions, substitutions, and/or insertions (e.g., synthetic deletions, substitutions, and/or insertions) can be made in a 3′ region of said promoter sequence and/or a 5′UTR, a 5′UTR intron, an exon sequence from a coding region, and/or an intron sequence from a coding region of the promoter sequence. The PsEF1A promoter homologs can be PsEF1Ap homologs (e.g., PsEF1A-0p, PsEF1A-1p PsEF1A-6p PsEF1A-7p), AhEF1Ap homologs, CaEF1Ap homologs, LaEF1Ap homologs, LjEF1Ap homologs, MtEF1Ap homologs, PvEF1A-4Ap, PvEF1A-4Bp, PvEF1Ap homologs, TpEF1Ap homologs, and VuEF1Ap homologs, e.g., any one of SEQ ID NOs: 1-4 and 9-46. The promoter sequence can share at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-4 and 9-39, and 43-46, and retain transcription initiation function. In some embodiments, the promoter sequence (which can include a 5′UTR, a 5′UTR intron, an exon sequence from a coding region, and/or an intron sequence from a coding region) comprises one or more deletions, substitutions, and/or insertions (e.g., synthetic deletions, substitutions, and/or insertions) relative to the nucleic acid sequence of PsEF1A-0p, PsEF1A-1p, PsEF1A-6p, PsEF1A-7p, which are set forth as SEQ ID NOs: 1-4, respectively. The promoter sequence can share at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with any one of SEQ ID NOs: 1-4, and retain transcription initiation function. For example, the promoter sequence can share at least 80% sequence identity with any one of SEQ ID NOs: 5-8, or comprise the nucleic acid sequence of any one of SEQ ID NOs: 5-8, and retain transcription initiation function.

Any polynucleotide of interest can be expressed in a plant or plant part according to the methods described herein, for expression driven by the promoter molecule of the present disclosure. For example, the coding sequence(s) may encode editing reagents (e.g., a guide

RNA, a nuclease) targeting any gene or genomic site of interest, a regulatory RNA, a small RNA, a selectable marker/reporter, an enzyme, a transcription factor, a receptor, a ligand, or a molecule that confers resistance to pests or disease, tolerance to herbicides, and/or advantageous agronomic traits (e.g., yield improvement, nitrogen use efficiency, water use efficiency, and nutritional quality).

The polynucleotide of interest according to the methods may encode a nuclease. The nuclease can be a CRISPR-associated Cas endonuclease. In specific embodiments, the CRISPR nuclease is a Cas12a (Cpf1) nuclease. In a specific embodiment, the Cas12a nuclease is a McCpf1 nuclease, e.g., a Mc.2Cpf1 2C-NLS nuclease. In some embodiments, the CRISPR nuclease shares at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with SEQ ID NO: 62 and retains nuclease activity, or comprises the sequence of SEQ ID NO: 62. In some embodiments, the nuclease is further operably linked to one or more nuclear localization sequences (NLSs) and/or one or more epitope tags. Any NLS or epitope tag can be linked to the nuclease provided herein. For example, the nuclease can be linked to a SV40-nucleophosmin 2 (SV40-NPM2) sequence. In some embodiments, the CRISPR nuclease with NLS shares at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity with SEQ ID NO: 63 and retains the nuclease activity and the NLS activity, or comprises the sequence of SEQ ID NO: 63.

The polynucleotide of interest according to the methods can encode a guide RNA. The polynucleotides of interest according to the methods can encode a guide RNA and a nuclease, each operably linked to a promoter molecule. Polynucleotides encoding a guide RNA and a nuclease can be both operably linked to the same promoter molecule of a DNA construct. Alternatively, polynucleotides encoding a guide RNA and a nuclease can be each operably linked to different promoter molecules, at least one of which is a promoter molecule of the present disclosure (e.g., a PsEF1Ap, a homolog, a fragment, or a derivative thereof e.g., with one or more deletions, substitutions, and/or insertions). Polynucleotides encoding a guide RNA and a nuclease can be contained in the same DNA construct or different DNA constructs.

In some embodiments, the polynucleotide of interest of the DNA construct operably linked to the promoter molecule encodes a guide RNA, and the plant or plant part further comprises, in operable linkage, a nucleic acid molecule encoding a second promoter molecule and a polynucleotide encoding a nuclease. In some embodiments, the second promoter molecule is an RNA polymerase II promoter molecule. The second promoter molecule operably linked to a nuclease can comprise a nucleic acid sequence that (i) comprises one or more deletions, substitutions, and/or insertions relative to the nucleic acid sequence of any one of SEQ ID NOs: 1-4, 9-39, and 43-46, shares at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%) sequence identity with any one of SEQ ID NOs: 1-4, 9-39, and 43-46, and retains transcription initiation function, (ii) has at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%) sequence identity to any one of SEQ ID NOs: 1-4, 9-39, 43-52, and 56-61, and retains transcription initiation function, or (iii) is any one of SEQ ID NOs: 1-4, 9-39, 43-52, and 56-61.

In some embodiments, the polynucleotide of interest of the DNA construct operably linked to the promoter molecule encodes a nuclease, and the plant or plant part further comprises, in operable linkage: a nucleic acid molecule encoding a second promoter molecule and a polynucleotide encoding a guide RNA. In some embodiments, the second promoter molecule is an RNA polymerase III (pol III) promoter molecule. The second promoter molecule operably linked to a guide RNA can comprise a nucleic acid sequence that (i) comprises one or more deletions, substitutions, and/or insertions relative to the nucleic acid sequence of any one of SEQ ID NOs: 1-4, 9-39, and 43-46, shares at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%) sequence identity with any one of SEQ ID NOs: 1-4, 9-39, and 43-46, and retains transcription initiation function, (ii) has at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%) sequence identity to any one of SEQ ID NOs: 1-4, 47-49, 51, and 53, and retains transcription initiation function, or (iii) is any one of SEQ ID NOs: 1-4, 47-49, 51, and 53.

Where the method comprises introducing a plant cell a DNA construct comprising polynucleotides encoding a nuclease and a guide RNA, exemplary combinations of promoters for polynucleotides encoding a nuclease and a guide RNA are indicated with “X” in Table 1. Each promoter in Table 1 is meant to include its variants and fragments, e.g., promoter molecules that share at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence similarity with the promoter molecule indicated, including promoter molecules comprising one or more deletions, substitutions, and/or insertions (e.g., synthetic deletions, substitutions, and/or insertions) thereof and retaining transcription initiation function. Combinations are not limited to those listed herein.

In some specific embodiments, the method of expressing a polynucleotide or interest in a plant or plant part comprises introducing into a plant, plant part, or plant cell a DNA construct comprising:
PsEF1A-6p operably linked to a polynucleotide encoding a nuclease and a gRNA;
PsEF1A-7p operably linked to a polynucleotide encoding a nuclease and a gRNA;
PsEF1A-0p operably linked to a polynucleotide encoding a nuclease and PsUBI3p operably linked to a polynucleotide encoding a gRNA;
PsEF1A-1p operably linked to a polynucleotide encoding a nuclease and PsUBI3p operably linked to a polynucleotide encoding a gRNA;
PsUBI3-SYN3p operably linked to a polynucleotide encoding a nuclease and
PsEF1A-0p operably linked to a polynucleotide encoding a gRNA;
PsUBI3-SYN3p operably linked to a polynucleotide encoding a nuclease and
PsEF1A-7p operably linked to a polynucleotide encoding a gRNA; or
AtUBI11p operably linked to a polynucleotide encoding a nuclease, and PsEF1A-1p operably linked to a polynucleotide encoding a gRNA.
   The method of transforming a plant or plant part can further comprise regenerating a plant or plant part from said plant cell.

Polynucleotides of interest according to the present disclosure can encode a selectable marker, a regulatory RNA, and/or a small RNA. The selectable marker, the regulatory RNA, and/or the small RNA can be operably linked to the promoter of the present disclosure, alone or together with another polynucleotide of interest (e.g., encoding a guide RNA or a nuclease), or any other promoter. For example, the regulatory RNA or small RNA can be operably linked to an RNA polymerase III promoter. Additionally or alternatively, the regulatory RNA or small RNA can be operably linked to a promoter molecule comprising a nucleic acid sequence that (i) comprises one or more deletions, substitutions, and/or insertions relative to the nucleic acid sequence of any one of SEQ ID NOs: 1-4, 9-39, and 43-46, shares at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%) sequence identity with any one of SEQ ID NOs: 1-4, 9-39, and 43-46, and retains transcription initiation function, (ii) has at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%) sequence identity to any one of SEQ ID NOs: 1-4, 47-49, 51, and 53, and retains transcription initiation function, or (iii) is any one of SEQ ID NOs: 1-4, 47-49, 51, and 53.

According to the methods described herein, the polynucleotide of interest can be stably inserted into the genome of the plant or plant part. The plant or the plant part can be stably transformed. Alternatively, the polynucleotide of interest can be transiently expressed in the plant or plant part, and the plant or the plant part can be transiently transformed.

In some embodiments, the polynucleotide of interest is constitutively expressed in the plant or plant part. In some embodiments, the polynucleotide of interest is expressed throughout the plant tissues and cells. In some embodiments, the polynucleotide of interest is expressed more strongly in certain tissues or cells, e.g., meristematic tissues or cells, compared to other tissues or cells. In some embodiments, the polynucleotide of interest is expressed in a developmentally-regulated manner. In some embodiments, the polynucleotide of interest is expressed upon induction via an inducible promoter of the present disclosure.

### C. Increasing Expression or Function of a Polynucleotide of Interest Operably Linked to the Promoter

In some embodiments, the methods of the present disclosure increase expression or function of one or more polynucleotides of interest or molecules encoded thereby operably linked to the promoter molecules in the plant or plant part, relative to a control plant or plant part, wherein the control plant or plant part comprises the polynucleotide(s) of interest operably linked to a control promoter. The control promoter, as used herein, does not comprise any one of: a nucleic acid sequence that has at least 75% (75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to any one of SEQ ID NOs: 1-46; or a nucleic acid sequence of any one of SEQ ID NOs: 1-46. The control promoter can be AtUBI11p, an exemplary nucleic acid sequence of which is set forth as SEQ ID NO: 47. The methods of the present disclosure can be used to express or enhance expression of any polynucleotide of interest, and are not limited to exemplary polynucleotides of interest described herein. For example, the polynucleotide(s) may encode editing reagents (e.g., a guide RNA, a nuclease), a regulatory RNA, a small RNA, a selectable marker / reporter, an enzyme, a transcription factor, a receptor, a ligand, or a molecule that confers resistance to pests or disease, tolerance to herbicides, and/or advantageous agronomic traits for expression or enhanced expression in a plant or plant part, according to the methods of the present disclosure. Editing reagents targeting any gene or genomic site of interest in a plant or plant parts can be used according to the methods of the present disclosure.

Expression or function of polynucleotide(s) of interest can be increased by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more with methods using a promoter of the present disclosure, as compared to using a control promoter. Expression or function of an operably linked polynucleotide of interest can be increased by at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more by using a promoter of the present disclosure compared to a control promoter. Gene or polynucleotide expression levels can be measured by any methods known in the art. For example, gene or polynucleotide expression levels can be measured by quantifying levels of the gene or polynucleotide product, e.g., an RNA or a protein, by, e.g., PCR, real-time PCR, Western blotting, and ELISA. Gene or polynucleotide expression levels can also be assessed by quantifying levels of function of gene or polynucleotide product, for example by quantifying the occurrence of events caused by the gene or polynucleotide product (e.g., editing frequency of a target gene) or by quantifying the levels of product produced by the gene or polynucleotide product.

The methods described herein can increase expression or function of polynucleotide(s) of interest as described above in a constitutive manner, and/or universally across various plant tissues (including but not limited to meristematic tissue), compared to methods using a control promoter. Additionally or alternatively, the methods can increase expression or function of polynucleotide(s) of interest as described above in a tissue-specific, tissue-preferred (e.g., meristem-preferred), cell-specific, cell-preferred (e.g., meristematic cell-preferred), developmental stage-specific, time-specific, and/or inducible manner compared to methods using a control promoter.

### D. Introducing Mutations to Plants

In some embodiments, methods and compositions of the present disclosure can be used to introduce mutations in the genome of a plant. Editing reagents targeting any gene or genomic site of interest in a plant or plant parts can be expressed from the promoters disclosed herein. Further, the embodiments disclosed herein are not limited to certain methods of introducing nucleic acids into a plant and are not limited to certain forms or structures that the introduced nucleic acids take. Any method of transforming a cell of a plant described herein with nucleic acids are also incorporated into the teachings of this innovation, and one of ordinary skill in the art will realize that the use of particle bombardment (e.g. using a gene-gun), Agrobacterium infection and/or infection by other bacterial species capable of transferring DNA into plants (e.g., Ochrobactrum sp., Ensifer sp., Rhizobium sp.), viral infection, and other techniques can be used to deliver nucleic acid sequences into a plant described herein. Methods disclosed herein are not limited to any size of nucleic acid sequences that are introduced, and thus one could introduce a nucleic acid comprising a single nucleotide (e.g. an insertion) into a nucleic acid of the plant and still be within the teachings described herein. Nucleic acids introduced in substantially any useful form, for example, on supernumerary chromosomes (e.g. B chromosomes), plasmids, vector constructs, additional genomic chromosomes (e.g. substitution lines), and other forms is also anticipated. It is envisioned that new methods of introducing nucleic acids into plants and new forms or structures of nucleic acids will be discovered and yet fall within the scope of the claimed invention when used with the teachings described herein.

Similarly, methods disclosed herein are not limited to certain techniques of mutagenesis. Any method of creating a change in a nucleic acid of a plant can be used in conjunction with the disclosed invention, including the use of chemical mutagens (e.g. methanesulfonate, sodium azide, aminopurine, etc.), genome/gene editing techniques (e.g. CRISPR-like technologies, TALENs, zinc finger nucleases, and meganucleases), ionizing radiation (e.g. ultraviolet and/or gamma rays) temperature alterations, long-term seed storage, tissue culture conditions, targeting induced local lesions in a genome, sequence-targeted and/or random recombinases, etc. It is anticipated that new methods of creating a mutation in a nucleic acid of a plant will be developed and yet fall within the scope of the claimed invention when used with the teachings described herein.

Introducing mutations into plants or plant parts to obtain desired traits may be achieved through the use of precise genome-editing technologies to modulate the expression of the endogenous sequence. In this manner, a nucleic acid sequence can be inserted, substituted, or deleted proximal to or within a native plant sequence encoding a polynucleotide of interest through the use of methods available in the art. Such methods include, but are not limited to, use of meganucleases designed against the plant genomic sequence of interest (D′Halluin et al (2013) Plant Biotechnol J 11: 933-941); CRISPR-Cas9, CRISPR-Cas12a (Cpf1), transcription activator-like effector nucleases (TALENs), zinc finger nucleases (ZFNs), and other technologies for precise editing of genomes [Feng et al. (2013) Cell Research 23:1229-1232, Podevin et al. (2013) Trends Biotechnology 31: 375-383, Wei et al. (2013) J Gen Genomics 40:281-289, Zhang et al (2013) WO 2013/026740, Zetsche et al. (2015) Cell 163:759-771, U.S. Provisional Patent Application 62/295,325]; N. gregoryi Argonaute-mediated DNA insertion (Gao et al. (2016) Nat Biotechnol doi:10.1038/nbt.3547); Cre-lox site-specific recombination (Dale et al. (1995) Plant J 7:649-659; Lyznik, et al. (2007) Transgenic Plant J 1:1-9; FLP-FRT recombination (Li et al. (2009) Plant Physiol 151:1087-1095); Bxb1-mediated integration (Yau et al. (2011) Plant J 701:147-166); zinc-finger mediated integration (Wright et al. (2005) Plant J44:693-705); Cai et al. (2009) Plant Mol Biol 69:699-709); and homologous recombination (Lieberman-Lazarovich and Levy (2011) Methods Mol Biol 701: 51-65; Puchta (2002) Plant Mol Biol 48:173-182).

In some aspects, inserting, substituting, or deleting one or more nucleotides at a precise location of interest may be achieved using a meganuclease or other suitable nuclease system designed to target the genomic sequence of interest. Without wishing to be bound by theory, a nuclease system can be used to achieve insertion, substitution, or deletion of genetic elements at a predefined genomic locus by causing a double-strand break at said predefined genomic locus and, optionally, providing an appropriate DNA template for insertion. This strategy is well-understood and has been demonstrated previously to insert a transgene at a predefined location in the cotton genome (D′Halluin et al. (2013) Plant Biotechnol J 11: 933-941). For example, a Cas12a (Cpf1) endonuclease coupled with a guide RNA (guide RNA) designed against the genomic sequence of interest can be used (i.e., a CRISPR-Cas12a system). Alternatively, a Cas9 endonuclease coupled with a guide RNA designed against the genomic sequence of interest (a CRISPR-Cas9 system), or a Cms1 endonuclease coupled with a guide RNA designed against the genomic sequence of interest (a CRISPR-Cms1) can be used. Other nuclease systems for use with the methods of the present invention include CRISPR systems (e.g., Type I, Type II, Type III, Type IV, and/or Type V CRISPR systems (Makarova et al 2020 Nat Rev Microbiol 18:67-83)) with their corresponding guide RNA(s), TALENs, zinc finger nucleases (ZFNs), meganucleases, and the like. Alternatively, a deactivated CRISPR nuclease (e.g., a deactivated Cas9, Cas12a, or Cms1 endonuclease) fused to a transcriptional regulatory element can be targeted to the upstream regulatory region of a polynucleotide of interest, thereby modulating the function of the polynucleotide of interest (Piatek et al. (2015) Plant Biotechnol J13:578-589).

Any editing reagents for use in any genome-editing methods including those described herein can be operably linked to the promoter of the present disclosure and expressed in a plant or plant part.

Methods disclosed herein include conferring desired traits to plants, for example, by mutating sequences of a plant, introducing nucleic acids into plants, using plant breeding techniques and various crossing schemes, etc. These methods are not limited as to certain mechanisms of how the plant exhibits and/or expresses the desired trait. In certain nonlimiting embodiments, the trait is conferred to the plant by introducing a nucleic acid sequence (e.g. using plant transformation methods) that encodes production of a certain protein by the plant. In certain nonlimiting embodiments, the desired trait is conferred to a plant by causing a null mutation in the plant's genome (e.g. when the desired trait is reduced expression or no expression of a certain trait). In certain nonlimiting embodiments, the desired trait is conferred to a plant by crossing two plants to create offspring that express the desired trait. It is expected that users of these teachings will employ a broad range of techniques and mechanisms known to bring about the expression of a desired trait in a plant. Thus, as used herein, conferring a desired trait to a plant is meant to include any process that causes a plant to exhibit a desired trait, regardless of the specific techniques employed.

In certain embodiments, a user can combine the teachings herein with high-density molecular marker profiles spanning substantially the entire genome of a plant to estimate the value of selecting certain candidates in a breeding program in a process commonly known as genome selection.

### E. Increasing Editing Efficiency at a Target Site

In some embodiments, methods of the present disclosure can be used to express editing reagents such as a guide RNA or a nuclease in a plant or plant part that are useful for introducing a mutation at a target site in the genome of a plant. Editing reagents targeting any gene or genomic site of interest in a plant or plant parts can be used, i.e., operably linked to a promoter of the present disclosure and introduced into a plant or plant part, according to the methods of the present disclosure. In such embodiments, the methods of the present disclosure can increase expression or function of editing reagents (e.g., a guide RNA and/or a nuclease) in the plant or plant part, relative to a control plant or plant part, wherein the control plant or plant part comprises the editing reagents (the guide RNA and/or the nuclease) and at least one of them are operably linked to a control promoter. Accordingly, in certain embodiments, the methods of the present disclosure can increase the efficiency or frequency of introducing a mutation to a genome of a plant or plant part, e.g., editing efficiency at a target site. Editing efficiency can be increased by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more by methods using a promoter of the present disclosure, as compared to a control plant or plant part comprising a control promoter. Editing efficiency can be increased by at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more by methods using a promoter of the present disclosure compared to a control promoter. In specific embodiments, the method of the present disclosure can increase an efficiency of introducing a mutation to a genome of a plant or plant part, e.g., editing efficiency at a target site of the editing reagents, is by about 10% to about 100% in the plant or plant part, resulting in about 1.1-fold to about 2-fold increase, relative to a control plant or plant part comprising a control promoter.

Additionally or alternatively, the methods of the present disclosure can increase the efficiency or frequency of introducing “fixed” changes (e.g., edits, mutations) to a genome of a plant or plant part relative to methods using a control promoter. A “fixed” change or a “fixed” edit event to a genome refers to a consistent (e.g., spatially consistent, ubiquitous) insertion-deletion profile across proliferating tissues in a mid-development T0 plant, where the change, edit, or mutation was introduced into a genome of a plant cell, by e.g., introducing editing reagents operably linked to promoters of the present disclosure into the plant cell, and stably transformed TO plants were regenerated from the plant cell. An “unfixed” change, an “unfixed” edit event, a “mosaic” change, or a “mosaic” edit to a genome refers to an inconsistent (e.g., spatially inconsistent) insertion-deletion profile within the TO plant tissues, where the change, edit, or mutation was introduced into a genome of a plant cell, by e.g., introducing editing reagents operably linked to promoters of the present disclosure into the plant cell, and the stably transformed T0 plant was regenerated from the plant cell. The frequency of fixed edit events over the total number of regenerated T0 plants can be increased by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, or by at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more by methods using a promoter of the present disclosure compared to a control promoter. The ratio of fixed edit events over unfixed edit events in regenerated T0 plants can be increased by about 10-100%, 20-100%, 30-100%, 40-100%, 50-100%, 60-100%, 70-100%, 80-100%, 20-90%, 30-90%, 40-90%, 50-90%, 60-90%, 70-90%, 100-1000%, 200-1000%, 300-1000%, 400-1000%, 500-1000%, 600-1000%, 700-1000%, 800-1000%, 200-900%, 300-900%, 400-900%, 500-900%, 600-900%, 700-900%, or more than 1000% (e.g., by about 10-20%, 20-30%, 30-40%, 40-50%, 50-60%, 60-70%, 70-80%, 80-90%, 90-100%, 100-200%, 200-300%, 300-400%, 400-500%, 500-600%, 600-700%, 700-800%, 800-900%, 900-1000%, or more than 1000%), e.g., by about 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more, or by at least 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, 1000%, or more by methods using a promoter of the present disclosure compared to a control promoter. Improved efficiency in introducing fixed edits into plants or plant parts according to the methods of the present disclosure can reduce chimerism and/or increase heritable editing in transformed and regenerated plants or plant parts.

The methods and compositions of the present disclosure can have an immediate, efficient induction of expression of operably linked polynucleotides of interest in plants or plant parts. When used to express editing reagents in plants, plant parts, or plant cells, the methods and compositions described herein can have an immediate, efficient induction of expression of editing reagents in transformed meristems and can enable editing to at a rate faster than their cellular division, thereby improving editing efficiency including efficiency of introducing fixed or heritable edits.

### VII. Breeding of Plants

Disclosed herein are methods for breeding a plant, such as a plant comprising a promoter molecule and/or a DNA construct of the present disclosure, or a plant generated according to the methods of the present disclosure. A plant containing the one or more heterogeneous nucleic acid sequences of the present disclosure may be regenerated from a plant cell or plant part, wherein the genome of the plant cell or plant part is genetically-modified to contain the one or more mutations or the polynucleotide of the present disclosure. Using conventional breeding techniques or self-pollination, one or more seeds may be produced from the plant that contains the one or more mutations or the polynucleotide of the present disclosure. Such a seed, and the resulting progeny plant grown from such a seed, may contain the one or more mutations or the polynucleotide of the present disclosure, and therefore may be transgenic. Progeny plants are plants having a genetic modification to contain the one or more mutations or the polynucleotide of the present disclosure, which descended from the original plant having modification to contain the one or more mutations or the polynucleotide of the present disclosure. Seeds produced using such a plant of the invention can be harvested and used to grow generations of plants having genetic modification to contain the one or more mutations or the polynucleotide of the present disclosure, e.g., progeny plants, of the invention, comprising the polynucleotide and optionally expressing a polynucleotide of agronomic interest (e.g., herbicide resistance gene).

Descriptions of breeding methods that are commonly used for different crops can be found in one of several reference books, see, e.g., Allard, Principles of Plant Breeding, John Wiley & Sons, NY, U. of CA, Davis, Calif., 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

### EXAMPLES

The following examples are offered by way of illustration and not by way of limitation. All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

### Example 1: Effect of PsEF1A-1p, PsEF1A-6p and PsEF1A-7p Linked to Guide RNA and/or CRISPR-Cas12a Nuclease in Transfected Pea Protoplasts

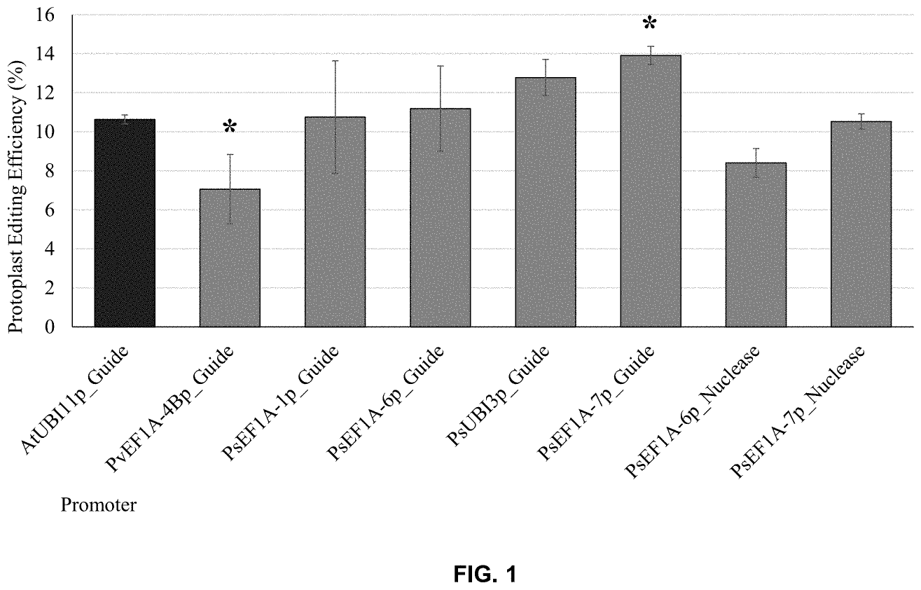

Promoters including Pisum sativum ELONGATION FACTOR 1-ALPHA 0, 6, and 7 promoters (PsEF1A-1p, PsEF1A-6p, PsEF1A-7p) were fused to a CRISPR-Cas12a (Cpf1) nuclease construct and a guide RNA construct in operable linkage, transfected in Pisum sativum cv. “Maxum” leaf protoplast cells using a standard polyethylene glycol process. The editing frequencies of the target gene site were determined after 24-hour incubation by a standard droplet digital PCR analysis. As shown in FIG. 1, PsEF1A-7p linked to the guide RNA showed a 1.3-fold higher editing frequency of a target compared to the control promoter (AtUBI11p) linked the guide RNA, demonstrating a statistically significant increase in editing efficiency (p=0.0001). On the other hand, the Phaseolus vulgaris ELONGATION FACTOR 4B promoter (PvEF1A-4Bp) linked to the guide RNA showed a 0.66-fold lower editing frequency of a target compared to the control promoter (AtUBI11p) linked the guide RNA, demonstrating a statistically significant decrease in editing efficiency (p=0.008). The nucleic acid sequences of PsEF1A-7p, PvEF1A-4Bp and AtUBI11p are set forth as SEQ ID NOs: 4, 36 and 47, respectively.

### Example 2: Effect of PsEF1A-1p and PsEF1A-7p Linked to CRISPR-Cas12a Nuclease in Transfected Tomato Protoplasts

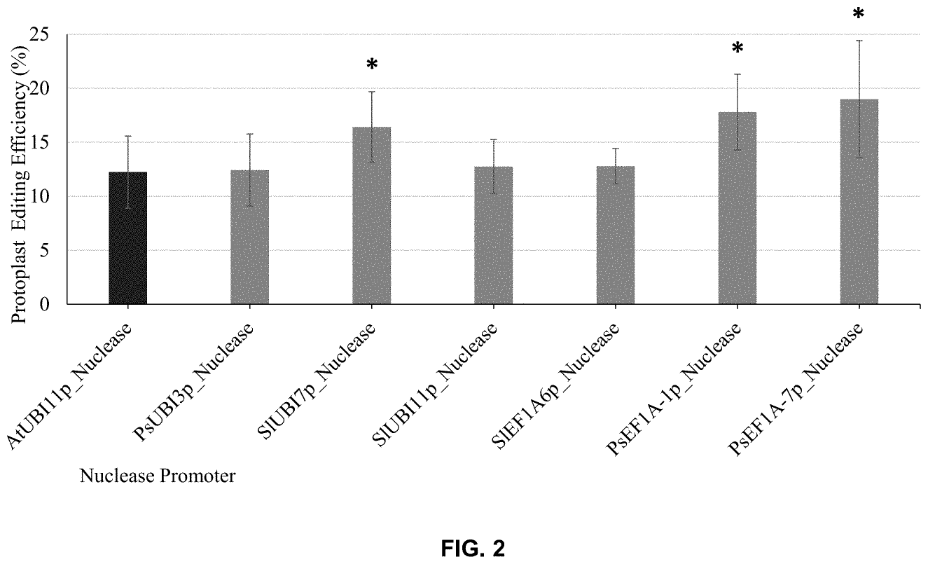

Promoters including PsEF1A-1p and PsEF IA-7p were fused to a CRISPR-Cas12a (Cpf1) nuclease construct in operable linkage, then transfected with a guide RNA construct in Solanum lycopersicon (tomato) cotyledon protoplast cells using a standard polyethylene glycol process. The editing frequencies of the target gene site were determined after 21-hour incubation by a standard droplet digital PCR analysis. As shown in FIG. 2, PsEF1A-1p and PsEF1A-7p respectively showed a 1.5- and 1.6-fold higher editing frequencies of a target compared to the control promoter (AtUBI11p), when each was linked to the CRISPR-Cas12a nuclease construct, demonstrating a statistically significant increase in editing efficiency (p=0.01, p=0.006, respectively). On the other hand, the tomato-derived SIEF1A6p showed no significant difference in editing frequencies of a target compared to the control promoter (AtUBI11p), when each were linked to the CRISPR-Cas12a nuclease construct. Tomato-derived SIUBI7p showed 1.3-fold significantly higher (p=0.02) editing frequencies of a target, whereas SlUBI11p showed no significant differences in such editing frequencies, each compared to the control promoter (AtUBI11p) when linked to the CRISPR-Cas12a nuclease construct. The nucleic acid sequences of the AtUBI11p (control), PsUBI3p, SlUBI7p SlUBI11p, SlEF1A6p, PsEF1A-1p, and PsEF1A-7p, promoters are set forth as SEQ ID NOs: 47, 48, 50, 54, 55, 2, and 4, respectively.

Effects of PsEF1A-0p, PsEF1A-1p, PsEF1A-7p, PsEF1A-0-SYN1p, PsEF1A-0-SYN2p, PsEF1A-1-SYN1p, and PsEF1A-1-SYN2p in nuclease expression and editing efficiency are tested in soybean protoplasts. The nucleic acid sequences of PsEF1A-0-SYN1p, PsEF1A-0-SYN2p, PsEF1A-1-SYN1p, and PsEF1A-0-SYN2p are set forth as SEQ ID NOs: 5-8, respectively.

### Example 3: Effect of PsEF1A-0p and PsEF1A-7p Linked to Guide RNA for Editing Stable Regenerated Soybean T0 Plants with a CRISPR-Cas12a Nuclease

Soybean (Glycine max) embryonic axes were stably transformed with constructs comprising promoters (AtUBI11p, PsEF1A-0p, PsEF1A-7p, GmScreamM4p, and/or PsUBI3-SYN3p) individually operably linked to a guide RNA and promoters (PsUBI3p, and/or PsUBI3-SYN3p) individually fused to a CRISPR-Cas12a (Cpf1) nuclease via Agrobacterium tumefaciens and editing frequencies at the target gene site was evaluated in T0 plants. The nucleic acid sequences for AtUBI11p, PsEF1A-0p, PsEF1A-7p, GmScreamM4p, PsUBI3p and PsUBI3-SYN3p are set forth as SEQ ID NOs: 47, 1, 4, 51, 48, and 49, respectively.

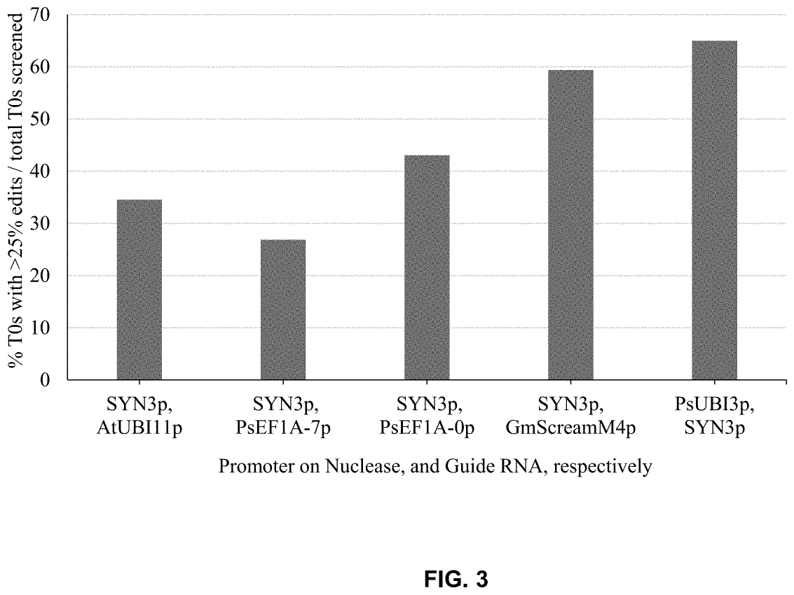

As shown in FIG. 3, PsEF1A-0p, PsEF1A-7p were associated with no significant difference in the percentages of T0 plants having >25% edits, as compared to the control promoter (AtUBI11p) and to another promoter (GmScreamM4p).

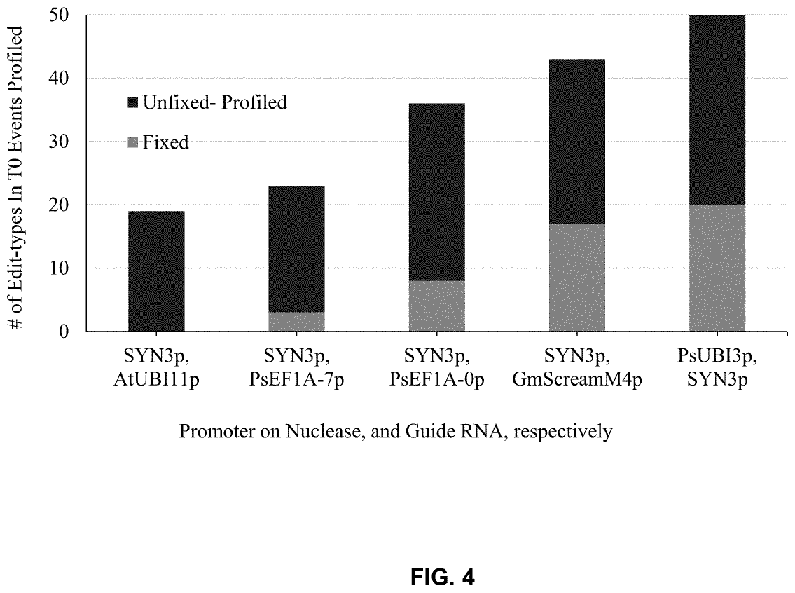

Furthermore, the incidence of overall editing events in T0 plants, which comprise the incidence of “fixed” edit events (i.e., a consistent insertion-deletion profile across proliferating tissues in a mid-development T0 plant) and the incidence of “unfixed” or mosaic edits (i.e., an inconsistent insertion-deletion profile within the T0 plant tissues) were analyzed across soybean plants stably transformed with guide RNA and a CRISPR-Cas12a nuclease each operably linked to various promoters as described above. As shown in FIG. 4, fixed edits were more likely to be heritable than mosaic edits, due to their occurrence inferably earlier than germline-establishment and seed-set. No significant differences were observed between the plants comprising PsEF1A-0p-, or PsEF1A-7p-guide RNA constructs, compared to the plants comprising GmScreamM4p-, or AtUBI11p-guide RNA constructs.

Put together, PsEF1A-0p and PsEF 1A-7p can be considered functionally equivalent as either the AtUBI11p or GmScreamM4p elements for guide RNA expression in stable regenerated soybean T0 editing, at least using the methodologies reported here.

Although the foregoing invention has been described in some detail by way of illustration and example for purposes of clarity of understanding, it will be obvious that certain changes and modifications may be practiced within the scope of the appended claims.

TABLE 2
Sequence Description

| SEQ | |
|---|---|
| ID | |
| NO: | DESCRIPTION |
| 1 | Nucleic acid sequence for PsEF1A-0p |
| 2 | Nucleic acid sequence for PsEF1A-1p |
| 3 | Nucleic acid sequence for PsEF1A-6p |
| 4 | Nucleic acid sequence for PsEF1A-7p |
| 5 | Nucleic acid sequence for PsEF1A-0-SYN1p |
| 6 | Nucleic acid sequence for PsEF1A-0-SYN2p |
| 7 | Nucleic acid sequence for PsEF1A-1-SYN1p |
| 8 | Nucleic acid sequence for PsEF1A-1-SYN2p |
| 9 | Nucleic acid sequence for AhEF1Ap |
| | (arahy.Tifrunner.gnm1.ann1.S49I6H) |
| 10 | Nucleic acid sequence for AhEF1Ap |
| | (arahy.Tifrunner.gnm1.ann1.PSFB2E) |
| 11 | Nucleic acid sequence for AhEF1Ap |
| | (arahy.Tifrunner.gnm1.ann1.08YRK9) |
| 12 | Nucleic acid sequence for AhEF1Ap |
| | (arahy.Tifrunner.gnm1.ann1.G03TES) |
| 13 | Nucleic acid sequence for CaEF1Ap (Ca_10593) |
| 14 | Nucleic acid sequence for CaEF1Ap (Ca_10594) |
| 15 | Nucleic acid sequence for CaEF1Ap (Ca_11433) |
| 16 | Nucleic acid sequence for CaEF1Ap (Ca_14697) |
| 17 | Nucleic acid sequence for CaEF1Ap (Ca_14698) |
| 18 | Nucleic acid sequence for CaEF1Ap (Ca_22296) |
| 19 | Nucleic acid sequence for LaEF1Ap (Lalb_Chr02g0141951) |
| 20 | Nucleic acid sequence for LaEF1Ap (Lalb_Chr02g0158111) |
| 21 | Nucleic acid sequence for LaEF1Ap (Lalb_Chr06g0174461) |
| 22 | Nucleic acid sequence for LaEF1Ap (Lalb_Chr14g0362911) |
| 23 | Nucleic acid sequence for LaEF1Ap (Lalb_Chr19g0129561) |
| 24 | Nucleic acid sequence for LaEF1Ap (Lalb_Chr21g0317711) |
| 25 | Nucleic acid sequence for LaEF1Ap (Lalb_Chr24g0403471) |
| 26 | Nucleic acid sequence for LjEF1Ap (Lj1g0010515) |
| 27 | Nucleic acid sequence for LjEF1Ap (Lj1g0026760) |
| 28 | Nucleic acid sequence for LjEF1Ap (Lj2g0002755) |
| 29 | Nucleic acid sequence for LjEF1Ap (Lj2g0008477) |
| 30 | Nucleic acid sequence for LjEF1Ap (Lj5g0021169) |
| 31 | Nucleic acid sequence for MtEF1Ap (Medtr4g014810) |
| 32 | Nucleic acid sequence for MtEF1Ap (Medtr6g021800) |
| 33 | Nucleic acid sequence for MtEF1Ap (Medtr6g021805) |
| 34 | Nucleic acid sequence for MtEF1Ap (Medtr1g013680) |
| 35 | Nucleic acid sequence for PvEF1A-4Ap (Phvul.004G060000) |
| 36 | Nucleic acid sequence for PvEF1A-4Bp (Phvul.004G075100) |
| 37 | Nucleic acid sequence for PvEF1Ap (Phvul.007G092500) |
| 38 | Nucleic acid sequence for TpEF1Ap |
| | (Tp57577_TGAC_v2_gene5210) |
| 39 | Nucleic acid sequence for TpEF1Ap |
| | (Tp57577_TGAC_v2_gene21632) |
| 40 | Nucleic acid sequence for TpEF1Ap |
| | (Tp57577_TGAC_v2_gene1766) |
| 41 | Nucleic acid sequence for TpEF1Ap |
| | (Tp57577_TGAC_v2_gene21641) |
| 42 | Nucleic acid sequence for TpEF1Ap |
| | (Tp57577_TGAC_v2_gene24200) |
| 43 | Nucleic acid sequence for VuEF1Ap (Vigun04g088900) |
| 44 | Nucleic acid sequence for VuEF1Ap (Vigun04g096500) |
| 45 | Nucleic acid sequence for VuEF1Ap (Vigun07g204100) |
| 46 | Nucleic acid sequence for VuEF1Ap (Vigun07g204200) |
| 47 | Nucleic acid sequence for AtUBI11p |
| 48 | Nucleic acid sequence for PsUBI3p |
| 49 | Nucleic acid sequence for PsUBI3-SYN3p |
| 50 | Nucleic acid sequence for SlUBI7p |
| 51 | Nucleic acid sequence for GmScreamM4p |
| 52 | Nucleic acid sequence for 35S-ENp |
| 53 | Nucleic acid sequence for AtU6-26p |
| 54 | Nucleic acid sequence for SlUBI11p |
| 55 | Nucleic acid sequence for SlEF1A6p |
| 56 | Nucleic acid sequence for AtUBI10p |
| 57 | Nucleic acid sequence for GmUBIp |
| 58 | Nucleic acid sequence for GmYAO-1p |
| 59 | Nucleic acid sequence for AtRPS5Ap |
| 60 | Nucleic acid sequence for GmDMC1p |
| 61 | Nucleic acid sequence for GmLAT52Lp |
| 62 | Amino acid sequence for McCpf1 |
| 63 | Amino acid sequence for McCpf1 with nuclear localization signal |
| | (NLS) |

1. A nucleic acid molecule comprising a promoter sequence, wherein the promoter sequence comprises one or more deletions, substitutions, and/or insertions relative to the nucleic acid sequence of any one of SEQ ID NOs: 1-4, 9-39, and 43-46; shares at least 75% sequence identity with any one of SEQ ID NOs: 1-4 and 9-39, and 43-46; and retains transcription initiation function.

2. The nucleic acid molecule of claim 1:
wherein said one or more deletions, substitutions, and/or insertions are located in a 3′ region of said promoter sequence; or
wherein the promoter sequence shares at least 80% sequence identity with any one of SEQ ID NOs: 5-8, or comprises the nucleic acid sequence of any one of SEQ ID NOs: 5-8, and wherein the promoter sequence retains transcription initiation function; or
wherein the nucleic acid molecule further comprises a 5′ untranslated region (UTR) sequence, a 5′UTR intron sequence, an exon sequence from a coding region, and/or an intron sequence from a coding region of a plant genome.

3-4. (canceled)

5. A DNA construct comprising, in operable linkage:
a. the nucleic acid molecule of claim 1, or a promoter molecule comprising a nucleic acid sequence that shares at least 80% sequence identity with any one of SEQ ID NOs: 1-4, 9-39, and 43-46, and retains transcription initiation function or a promoter molecule comprising a nucleic acid sequence of any one of SEQ ID NOs: 1-4, 9-39, and 43-46; and
b. a polynucleotide of interest.

6. The DNA construct of claim 5:
wherein the DNA construct comprises, in operable linkage:
a. a promoter molecule comprising a nucleic acid sequence that shares at least 80% sequence identity with any one of SEQ ID NOs: 1-4, and retains transcription initiation function, or a promoter molecule comprising a nucleic acid sequence of any one of SEQ ID NOs: 1-4; and
b. a polynucleotide of interest, or
wherein the promoter molecule further comprises a 5′ untranslated region (UTR) sequence, a 5′UTR intron sequence, an exon sequence from a coding region, and/or an intron sequence from a coding region of a plant genome.

7. (canceled)

8. The DNA construct of claim 5, wherein the polynucleotide of interest encodes a guide RNA and/or a nuclease.

9. The DNA construct of claim 8:
(i) wherein the polynucleotide of interest encodes a guide RNA, and wherein the DNA construct further comprises, in operable linkage:
a. the nucleic acid molecule of any one of claims 1-4; a promoter molecule comprising a nucleic acid sequence that has at least 80% sequence identity to any one of SEQ ID NOs: 1-4, 9-39, 43-52, and 56-61, and retains transcription initiation function; or a promoter molecule comprising a nucleic acid sequence of any one of SEQ ID NOs: 1-4, 9-39, 43-52, and 56-61; and
b. a polynucleotide of interest encoding a nuclease; or
(ii) wherein the polynucleotide of interest encodes a nuclease, and wherein the DNA construct further comprises, in operable linkage:
c. the nucleic acid molecule of any one of claims 1-4; a promoter molecule comprising a nucleic acid sequence that has at least 80% sequence identity to any one of SEQ ID NOs: 1-4, 47-49, 51, and 53, and retains transcription initiation function; or a promoter molecule comprising a nucleic acid sequence of any one of SEQ ID NOs: 1-4, 47-49, 51, and 53; and
d. a polynucleotide of interest encoding a guide RNA.

10-13. (canceled)

14. The DNA construct of claim 8, wherein the nuclease is a clustered, regularly interspaced, short palindromic repeats (CRISPR)-associated Cas endonuclease, or the nuclease is further operably linked to one or more nuclear localization sequences (NLSs) and/or one or more epitope tags.

15. The DNA construct of claim 5, comprising a nucleic acid molecule encoding a selectable marker, morphogen gene, reporter gene, and/or a regulatory RNA, operably linked to a promoter molecule.

16. The DNA construct of claim 15, wherein the promoter molecule operably linked to the regulatory RNA comprises: a nucleic acid molecule comprising one or more deletions, substitutions, and/or insertions relative to the nucleic acid sequence of any one of SEQ ID NOs: 1-4, 9-39, and 43-46, shares at least 75% sequence identity with any one of SEQ ID NOs: 1-4 and 9-39, and 43-46, and retains transcription initiation function; a nucleic acid sequence that has at least 80% sequence identity to any one of SEQ ID NOs: 1-4, 47-49, 51, and 53, and retains transcription initiation function; or a nucleic acid sequence of any one of SEQ ID NOs: 1-4, 47-49, 51, and 53.

17. A cell comprising the nucleic acid molecule of claim 1 or a DNA construct comprising, in operable linkage, said nucleic acid molecule of claim 1 and a polynucleotide of interest.

18-19. (canceled)

20. A plant or plant part comprising, in operable linkage:
a. a nucleic acid molecule comprising a promoter sequence, wherein the promoter sequence comprises one or more deletions, substitutions, and/or insertions relative to the nucleic acid sequence of any one of SEQ ID NOs: 1-4, 9-39, and 43-46; shares at least 75% sequence identity with any one of SEQ ID NOs: 1-4, 9-39, and 43-46; and retains transcription initiation function;
a promoter molecule comprising a nucleic acid sequence that has at least 80% sequence identity to any one of SEQ ID NOs: 1-4, 9-39, and 43-46, and retains transcription initiation function; or
a promoter molecule comprising a nucleic acid sequence of any one of SEQ ID NOs: 1-4, 9-39, and 43-46; and
b. a polynucleotide of interest.

21. The plant or plant part of claim 20:
wherein said one or more deletions, substitutions, and/or insertions are located in a 3′ region of said promoter sequence; or
wherein the promoter sequence shares at least 80% sequence identity with any one of SEQ ID NOs: 5-8, or comprises the nucleic acid sequence of any one of SEQ ID NOs: 5-8, and retains transcription initiation function; or
wherein the nucleic acid molecule comprising a promoter sequence or the promoter molecule comprises a 5′ untranslated region (UTR) sequence, a 5′UTR intron sequence, an exon sequence from a coding region, and/or an intron sequence from a coding region of a plant genome; or
wherein the nucleic acid molecule, the DNA construct, or part thereof is stably inserted in the genome of said plant or plant part; or
wherein said plant or plant part is a legume.

22-26. (canceled)

27. A method of expressing a polynucleotide of interest in a plant or plant part comprising introducing a DNA construct into the plant or plant part, wherein the DNA construct comprises, in operable linkage:
a. a nucleic acid molecule comprising a promoter sequence, wherein the promoter sequence comprises one or more deletions, substitutions, and/or insertions relative to the nucleic acid sequence of any one of SEQ ID NOs: 1-4, 9-39, and 43-46; shares at least 75% sequence identity with any one of SEQ ID NOs: 1-4, 9-39, and 43-46; and retains transcription initiation function;
a promoter molecule comprising a nucleic acid sequence that has at least 80% sequence identity to any one of SEQ ID NOs: 1-4, 9-39, and 43-46, and retains transcription initiation function; or
a promoter molecule comprising a nucleic acid sequence of any one of SEQ ID NOs: 1-4, 9-39, and 43-46; and
b. a polynucleotide of interest.

28. The method of claim 27, comprising:
introducing the DNA construct into a plant cell; and
regenerating a transformed plant or plant part from said plant cell.

29. The method of claim 27:
wherein said one or more deletions, substitutions, and/or insertions are located in a 3′ region of said promoter sequence; or
wherein the promoter sequence shares at least 80% sequence identity with any one of SEQ ID NOs: 5-8, or comprises the nucleic acid sequence of any one of SEQ ID NOs: 5-8, and retains transcription initiation function; or
wherein the nucleic acid molecule comprising the promoter sequence or the promoter molecule further comprises a 5′ untranslated region (UTR) sequence, a 5′UTR intron sequence, an exon sequence from a coding region, and/or an intron sequence from a coding region of a plant genome.

30-32. (canceled)

33. The method of claim 27, wherein the polynucleotide of interest encodes a guide RNA and/or a nuclease.

34. The method of claim 27:
(i) wherein the polynucleotide of interest encodes a guide RNA, and wherein the DNA construct further comprises, in operable linkage:
a. the nucleic acid molecule of any one of claims 1-4; a promoter molecule comprising a nucleic acid sequence that has at least 80% sequence identity to any one of SEQ ID NOs: 1-4, 9-39, 43-52, and 56-61, and retains transcription initiation function; or a promoter molecule comprising a nucleic acid sequence of any one of SEQ ID NOs: 1-4, 9-39, 43-52, and 56-61; and
b. a polynucleotide of interest encoding a nuclease; or
(ii) wherein the polynucleotide of interest encodes a nuclease, and the DNA construct further comprises, in operable linkage:
a. the nucleic acid molecule of any one of claims 1-4; a promoter molecule comprising a nucleic acid sequence that has at least 80% sequence identity to any one of SEQ ID NOs: 1-4, 47-49, 51, and 53, and retains transcription initiation function; or a promoter molecule comprising a nucleic acid sequence of any one of SEQ ID NOs: 1-4, 47-49, 51, and 53; and
b. a polynucleotide of interest encoding a guide RNA; or
(iii) wherein the polynucleotide of interest encodes a nuclease, and the nuclease is further operably linked to one or more nuclear localization sequences (NLSs) and/or one or more epitope tags; or
(iv) wherein the DNA construct further comprises a nucleic acid molecule encoding a selectable marker and/or a regulatory RNA, operably linked to a promoter molecule; or
(v) wherein the nucleic acid molecule, the DNA construct, or part thereof is stably inserted in the genome of said plant or plant part.

35-42. (canceled)

43. The method of claim 27:
wherein expression or function of one or more molecules encoded by the polynucleotide(s) of interest is increased in the plant or plant part relative to a control plant or plant part comprising the polynucleotide(s) of interest operably linked to a control promoter that does not comprise the promoter molecule of claim 27, or
wherein the polynucleotide(s) of interest encodes a guide RNA and/or a nuclease, and wherein an efficiency of introducing a mutation to a genome of the plant or plant part is increased relative to the control plant or plant part; or
wherein the polynucleotide(s) of interest encodes a guide RNA and/or a nuclease, and wherein an efficiency of introducing a heritable mutation to a genome of a stably transformed and regenerated plant or plant part consistently across plant tissue is increased relative to the control plant or plant part.

44-46. (canceled)

47. The method of claim 27, wherein said plant or plant part is a legume.

48. A plant or plant part produced by the method of claim 27, wherein said plant or plant part comprises said DNA construct or part thereof.


SEQUENCE LISTING (63 sequences)

SEQ ID NO: 1 - DNA, 2435 bp, Pisum sativum
  source 1..2435: mol_type = genomic DNA
agtgtctgtgaacacaaacaattacaacattatggttttaaggttgcattttatggttttatggtttcaatgtcaacacggttcaatgtaacaagattatatttgtaatcatgttaatgcaaccaactttgtttacttcgatattattctcatgacaaataccaactttgtatcatatttggtctgataaatgacttaccacaatacatgaagaatttactccatgtcaccaaacataacatgtctaataaataatataattgattcctttagttactttaatgttttaatttagtcccttatttattaaacggaacattttgagtccctaaactaataagttttgtttcaaaacgatttattaattgcaatttttctcaaaatagcacataacacaaattatgctatagtagagtaaaacattaacacatattttatggatttcaaacaaacctaaaatttatataacaaacacatgaattacatatatttcaaccaacgcatatgatcaactaaccctatttgaaatacattgttaaaaacactccattcaaccctaaactaaaaatgctaaatacatgacattttcaacattcctcttgtgtggacaacctcagtctttaacttgaaaaatttattcttttgcctttcaatcttcaaattccatccatcaacaatttcatcacataaccacttgaaaaaattacattccttttccatttgatttatgtaattctacaactctataacttttttctaacattcacaatgttcatatatgtaactataagaagaacacacccctcgtggcaaaaatactccctcttcttgttttttttagcacaaatccaaatatcccataattaccgaaacaatgtaacatttgaacataaattttgaaattatgaaaaaaaatagtagaatattgatataagatatttgttgagttaggaatttaaattttgccttggagtcatgctacatcatcctacattaatatatttttttcaaaatttaaacaaaaaaaattaaattggctaacaaaattaagattaacttaaaattaacttaatacttcgccactaattaattcttagtaaattcaattcaaaattttgtagttgcttaatagatatggaactcgaaatctttcatatgaaaaaactcactaaagcatattctcacgcgtacagacggcgtgtgacttcaaactccaagaaggacctagaagaaaatccacgagagaaatacagtgtacacacgtatttccttatgagggtgaaaaaacgcaacgttcaaataaacaaaccgagggcatttcaggaaattcataagaaaaccaaggataatattggaatataaaaaatatttttctcactataaaaaaacttgaaccctagttatttcattaccactctttacttgttcttaaatagctcagtcactctgcggctgaggattccgaacgagttttcttcgatttcatttctgctgaggtaaaattcttcgattcatttctctttgattcgatctattgtgtttatttgctgtagtatcatatgtatgattttgttaggttagtttgattttggttaattttgtttagtgtttgagaaatttgtattttgaacgagtcgatttgtgttcgtaatctatgatttaatttagctaattgagtcgattggttttctgtatgtatagatctgatgaattttggttcaaatcaagtaatttgattttgaaagttatgaactttttgtgttatggttgtttaattatggatctgttaaagattagactctgattcaattgatgattgtttaattgatgaaaatgatattttttttgatggagatttgattgtctctttctgatactttaagcagtggcatattttaccttcaaaattttttattttctattactcgtttttatatgctataacacgagttgttgatatgtttcgaaatttttaaagttgtttattttgagttaatttttctgattaattggttagtttggaggtaagactcttatttaatcagggtttgttttggtttgtttatgatgcaatgttttgatttaactgttttattagatcttatttgtgtatctggttttgaatttgttgctgttgatttctagcttattagttttgaacttcataacatgttcaattgctgttctttagttaatatgatttattagttatgaccatgttgaattgctgttctttttgcttattagttctgacttttgaatttctttttgtgaattgcagttaagacatcatgggaaaggaaaaggttcatatcaacattgtcgtcattggacatgtcgactcaggaaaatcaaccaccactggtcacttgatctacaagctaggaggtattgacaagcgtgtgattgagagatttgagaaggaagcagctgag

SEQ ID NO: 2 - DNA, 2331 bp, Pisum sativum
  source 1..2331: mol_type = genomic DNA
tggaagcccgtgttcgatggtgcctcaaatgcaatagggcatgaaattgttgtcgtcttgatcaaaagaatatgcttcttaaaattgaaacaagatgtttatagcttaagactaatgatttaaagcttcaaaaagaatatgtttcaaaatctaagtaagatcatcattccattccttaggtacatgcttgacttggatccaatctagcaccttgcaccagataaacctcaattcaagtacatccaaaaacacaagtgattgatgtctcaatctcaggacatgaccaacatttgtctgtcagacattccaaacacctgtagtctagcctttatgcttgccatgacacacaagccataagatcatctgcgatctaagcctaactttgttgccatagaataatttcctccaattgactttaggttcctcatctctcaaggccaagtacatgactttcgtggtgaattttctcctatgcatcatttgctcccaacactgcaaattttcaatactctccctcaactgcagaatgactttcattatccaaaagtaactagttttggtcttcacatccatcaaggtgttatgtttaatgtaataggtggaaattcatttcatccataagttatcggattttccatagaggttccaaagcagcttcaacatagtagcctccgttccatttctccaactcaatgattctcatcccaccttggatcttaggtctgcacacattcttccacgcaacagagctcttcctgctaatcttaacacttccaatctacatgaaagacatgcatatactctcaattttatggatcacatgtttagggaaaagaaagcattacatctagtaattagagatgacaaaagtcacactattgattagctggattctctatgcatatctcaacaacttagtactccaatgtttgattcttcctacaatcttatcaacaagattcatgtaatgatgaatgaccaactttttgctagtcaaaggcacccataaatatccgaaaggtaacaaaagtagtcaaatttttgatgcactactttatgatgttatctactccaccaaaataaattttacatttatttggattcatcttaaggtcagagacagaaaacggatgaacgtattcatcataaattcaatagatctaagatccactaacaaacttaccgcaaattatttctattgttatttctatcggatcactaacataataaagtatatttatttaccaagacaagatggaattaaacaaattttagatagcaagtatattagatatttttgggcatttaatttgggccttggtcgaacaagaacccaaactatgctgtcaatttagcaaacctcaacaaaacgacgtcgttccaatcttcaatttcatccttacctaaaacctaactataaaaatactgaaaccctaacatttatcactctctcaaaacttgttctcgattgctcagtctctctctgcggctgaggattccgaacgagtgacatacttcaatttcgtttctgctaaggtaacatccttcaacttctgtctctctgattcgatctatgctctaagattcttatttgctgtcgtatactatgactctgttagatgattttgttcggttgttttattttatttcattgaatttgtaaactttcttgtgtatgttcggtgatgctttaatcagttgcatgctttacttgtgaaaagttttgatctgagtcctgtttatgatttatcatagttggatctagttgatatgttttaatctcttaaacattgtagactgaatgttgtttcttatgtatttattagtttctctatttaattgataattggggttttctgataattgaatttgttttgttgctctgctaaggattttggttcatctgttagaatggatcttatttggttaagctgattttgaacatgatgctgtttgctgttaactgttaattatcaaagttgggtttttatgatctgaatggtgcttttagctgtttatctgatcttgagcttgttgttgtttactgtttgtctgattttgatagtgatttagcttgttgtttttctgtctttattaatcattgctttgttgttaacatgaattatgagaacttacttttggaacttttttgattttgctgttcagttattattttatacttgtttttgatgttcatctgattttgaacttgatctattgttattttttctgggactaactattattgttattttgtttatgctgttcagtcattattattttatattagatctgacatgtgaagcttctgtgaattgcagttaagacatc

SEQ ID NO: 3 - DNA, 1897 bp, Pisum sativum
  source 1..1897: mol_type = genomic DNA
gggatgttataagtagcatcatgcttgaaggacatatgtgttatatatgatttatttatttatttgtcgagtcgggaagtagggtgttacaggtagcatgcacacctgtaccttttgaccattacaatgacatccaccgtggaatttcattaaaactaacatggtacacacctctgtcaccataactcccgccatcttctttatgaagatggttgacaagactctatattctaacacaaacaccattgtcagaggtttcgttggtggagataactctagcttcccgaagaaaacacacaaagcaggtgcttgcaataaatgtaatatctcttggtttcccaagcggcgcttgagggaaaatagggggttatcatcaccttttccaaggaggacaccctcatagtcacaactcgttatcaaacgagtattgatagatcctggtaactcaactgatatcctattctaggacgcctctcaaaagttgcagatcaatcatgacgtcaaaatgttcaacgactctttaacaggattgtcatgcgaacaagtataagtaatgactcatatgaccctggagaccacatgtggcgagggtgcaaacgctaacgcgattacggtcagctactttattaaaaaataatttatcatttaaaaattagacatattaattttaaatacaaaagtataagaggagatcaaaactattaaatttaaaatagaagaattactttcgttaataaaattaaataataggcaaaactgtaattaaacttaaataaaaattaaataaataaaaaataagaaaaaaatggaataaatgatgattataaaagattactattgcaaattaccgtcctattatatataaaagataaataaaacgtaaaataaatgatgatttatgagataaattagtgctgcaaattaattttttatacataaaatattttttattcgatatcaatttttgtggaattgtataatgcatttacactatttatcattattaaataattaataaacattttttttataaataataatgataataataatagtaaaaaatatttaattaattaatattttgatttgaataaagtaaagtgtattgtaaaagatttgatttttctgtagaacataaataaataataaatatttaaaaattatatttgattcggtaattcacgaaaactatgctcccgtcctttataaaccctccaacccttcttttgttaacactacactcttttctcattctcctccctgcggctgtgcattagggcatttttctccttcaaggtaaatcattctccgatttcgtttgttaatcgtgtatcagtatcatcgttaccatcgcgattcatagggttttctcacattttagtgtatatgtatgtgattacgtaggatcatttgatattagcatgttattgattattgttttgctttccgatttgttattcgattccgaaatctgcctagggtttacgtgcctgtttaactgttattctgaatccgaatagcgtgttaatcgattatgcttttgaatcggttgttttttttgtaaaactacatatggctattgatattttccagagatttttctgttgtgattcttgatgcatatcatcataatagacggaaatctatggatttatataatgctaaatatgattgtgtttgttttctgatcatgttgtgttcttgatctgctgagatatgttgtggtgtattgtctaatttatagtgtaattgttgacatggattttgcatatttgatttgtcagaatgttgttgatgctgttttatttaattttcttttttaatgctggtgtttattttacgtgtgttgttttctgcttttgatttgtagattttgtggttttaagctttagttcaa

SEQ ID NO: 4 - DNA, 2036 bp, Pisum sativum
  source 1..2036: mol_type = genomic DNA
aggtttataatcgtcctttacataacctagaataacatgggcttcatgctaaataattcgcagatccaaatcaaattgctgcgcaatcttctgcagaattaaattaaatcaaatcaaatcagatcaaatcaaatctgatgtgtccttaaatcttttaacatcctttcttaggaaataaagcaaagcttaaactgacctttgcttctaaaacaaacgcttattggaccgcataatgcttaaaaataaatattccaagaatgtaaaaccacaaaacaccacgatgtcaagacatcttgttcaacatcttgctatttcacttgttttaacaaaatactgtcaatcacaatacaaacaacctaacaatattgacgtgaagcaaacaaagacttcaacctcaaagagcgggcaacaagtaaaggtttaatcactcgatataactaaatgatcattcctttaaaagcaatcaaccaatagttctcttttagtagaactacgtagccttgagttctccatagcacctagagatacgtatgagtgcgattgagaaatcttatcagacacattaatgttaaaaaacacccatttaataaaaatacatcttcctttctttttctcgctcagtaagtagaatgtaacatttaacattagctaacactctattacaaatctaactaagtggttctcattgagtacaacggatgtgaaaggtgttgataccttccccttgcataaccgaccctcgaatccgatttggttgcgatagtcatttcttttttgtttttatgggttttatcaatattttctctttccctctttgtgaataaataaaattcggtggcaactctattcaaacatctttttcgcgagcgtgcgagcgcctcgcggatgattgcattttcaagatgcgacaagttgatgatgatatatcatggttgcgtatggtataagaacctgtcattaaagacctggtgtgttattcggactccaactctaatactcctatcaaacttatctcaaagtttactaatttttaacatcactatctcacgattcttcaaaacatcaaagagcttctcaccagagaatgacaattttatatatttcataatcttagagagaagaatgcgtgtgctgactatttagcaaaacatgaggctgataatgatacaacatatcaatcttttgcggagcctcctctaaaaatcaccactcttctactagctgatgctaatgagactttactttttagataaattcttattttttttctttttctttttccgcttaaaaaaaacattttttataagatttaaaatcaaataattttttataatttaatataattttattttaaaaaaaaaggcatcttcttttaatgaaattgacaaccaagaaaaatcaaaatattattgcatctattaaatgattttgttatcataaataaaatttattggatgtcttttggtcaactcacttccttaacctagtctagacataaccctaaaaacaaggcttccttctaaatttgttggccgccaaacgaataaggtttagataggaaaacagagggcaattccgtccaaataaataaacacagctcatactactatatataaaccctaatactgtaactcttctctactctcatttccttctttgcggctagctttttccttcaaggtaaaatctcttctatttcgtttctaattcatgtcttgttcttatcatttactttagatttgctggttcgttattatatgattgataatcatgctgagatttatgtccctgtttaatatttattctgaatctaaatagtgttataatcgtttatgtttttgaatctgtagttttctttggtaccaaggtattagatgtaggtgttatgcaaatttggtttgttagaatagttttgtggctagtatttttatttatgtgttgtgtcatgtgttgaaacggtagatcttctgtgtggtaatggtggttttacgtgtttttttttggcagatcttgtgtaattttaagcttaagcacag

SEQ ID NO: 5 - DNA, 1862 bp, synthetic construct
  source 1..1862: mol_type = other DNA
agtgtctgtgaacacaaacaattacaacattatggttttaaggttgcattttatggttttatggtttcaatgtcaacacggttcaatgtaacaagattatatttgtaatcatgttaatgcaaccaactttgtttacttcgatattattctcatgacaaataccaactttgtatcatatttggtctgataaatgacttaccacaatacatgaagaatttactccatgtcaccaaacataacatgtctaataaataatataattgattcctttagttactttaatgttttaatttagtcccttatttattaaacggaacattttgagtccctaaactaataagttttgtttcaaaacgatttattaattgcaatttttctcaaaatagcacataacacaaattatgctatagtagagtaaaacattaacacatattttatggatttcaaacaaacctaaaatttatataacaaacacatgaattacatatatttcaaccaacgcatatgatcaactaaccctatttgaaatacattgttaaaaacactccattcaaccctaaactaaaaatgctaaatacatgacattttcaacattcctcttgtgtggacaacctcagtctttaacttgaaaaatttattcttttgcctttcaatcttcaaattccatccatcaacaatttcatcacataaccacttgaaaaaattacattccttttccatttgatttatgtaattctacaactctataacttttttctaacattcacaatgttcatatatgtaactataagaagaacacacccctcgtggcaaaaatactccctcttcttgttttttttagcacaaatccaaatatcccataattaccgaaacaatgtaacatttgaacataaattttgaaattatgaaaaaaaatagtagaatattgatataagatatttgttgagttaggaatttaaattttgccttggagtcatgctacatcatcctacattaatatatttttttcaaaatttaaacaaaaaaaattaaattggctaacaaaattaagattaacttaaaattaacttaatacttcgccactaattaattcttagtaaattcaattcaaaattttgtagttgcttaatagatatggaactcgaaatctttcatatgaaaaaactcactaaagcatattctcacgcgtacagacggcgtgtgacttcaaactccaagaaggacctagaagaaaatccacgagagaaatacagtgtacacacgtatttccttatgagggtgaaaaaacgcaacgttcaaataaacaaaccgagggcatttcaggaaattcataagaaaaccaaggataatattggaatataaaaaatatttttctcactataaaaaaacttgaaccctagttatttcattaccactctttacttgttcttaaatagctcagtcactctgcggctgaggattccgaacgagttttcttcgatttcatttctgctgaggtaaaattcttcgattcatttctctttgattcgattctgattcaattgatgattgtttaattgatgaaaatgatattttttttgatggagatttgattgtctctttctgatactttaagcagtggcatattttaccttcaaaattttttattttctattactcgtttttatatgctataacacgagttgttgatatgtttcgaaatttttaaagttgtttattttgagttaatttttctgattaattggttagtttggaggtaagactcttatttaatcagggtttgttttggtttgtttatgatctacaagctaggaggtattgacaagcgtgtgattgagagatttgagaaggaagcagctgag

SEQ ID NO: 6 - DNA, 2435 bp, synthetic construct
  source 1..2435: mol_type = other DNA
agtgtctgtgaacacaaacaattacaacattatggttttaaggttgcattttatggttttatggtttcaatgtcaacacggttcaatgtaacaagattatatttgtaatcatgttaatgcaaccaactttgtttacttcgatattattctcatgacaaataccaactttgtatcatatttggtctgataaatgacttaccacaatacatgaagaatttactccatgtcaccaaacataacatgtctaataaataatataattgattcctttagttactttaatgttttaatttagtcccttatttattaaacggaacattttgagtccctaaactaataagttttgtttcaaaacgatttattaattgcaatttttctcaaaatagcacataacacaaattatgctatagtagagtaaaacattaacacatattttatggatttcaaacaaacctaaaatttatataacaaacacatgaattacatatatttcaaccaacgcatatgatcaactaaccctatttgaaatacattgttaaaaacactccattcaaccctaaactaaaaatgctaaatacatgacattttcaacattcctcttgtgtggacaacctcagtctttaacttgaaaaatttattcttttgcctttcaatcttcaaattccatccatcaacaatttcatcacataaccacttgaaaaaattacattccttttccatttgatttatgtaattctacaactctataacttttttctaacattcacaatgttcatatatgtaactataagaagaacacacccctcgtggcaaaaatactccctcttcttgttttttttagcacaaatccaaatatcccataattaccgaaacaatgtaacatttgaacataaattttgaaattatgaaaaaaaatagtagaatattgatataagatatttgttgagttaggaatttaaattttgccttggagtcatgctacatcatcctacattaatatatttttttcaaaatttaaacaaaaaaaattaaattggctaacaaaattaagattaacttaaaattaacttaatacttcgccactaattaattcttagtaaattcaattcaaaattttgtagttgcttaatagatatggaactcgaaatctttcatatgaaaaaactcactaaagcatattctcacgcgtacagacggcgtgtgacttcaaactccaagaaggacctagaagaaaatccacgagagaaatacagtggtgacacgtggcagcttatgagggtgaaaaagtgacacgtggcagtaaacaaaccgagggcatttcaggaaattcataagaaaaccaaggataatattggaatataaaaaatatttttctcactataaaaaaacttgaaccctagttatttcattaccactctttacttgttcttaaatagctcagtcactctgcggctgaggattccgaacgagttttcttcgatttcatttctgctgaggtaaaattcttcgattcatttctctttgattcgatctattgtgtttatttgctgtagtatcatatgtatgattttgttaggttagtttgattttggttaattttgtttagtgtttgagaaatttgtattttgaacgagtcgatttgtgttcgtaatctatgatttaatttagctaattgagtcgattggttttctgtatgtatagatctgatgaattttggttcaaatcaagtaatttgattttgaaagttatgaactttttgtgttatggttgtttaattatggatctgttaaagattagactctgattcaattgatgattgtttaattgatgaaaatgatattttttttgatggagatttgattgtctctttctgatactttaagcagtggcatattttaccttcaaaattttttattttctattactcgtttttatatgctataacacgagttgttgatatgtttcgaaatttttaaagttgtttattttgagttaatttttctgattaattggttagtttggaggtaagactcttatttaatcagggtttgttttggtttgtttatgatgcaatgttttgatttaactgttttattagatcttatttgtgtatctggttttgaatttgttgctgttgatttctagcttattagttttgaacttcataacatgttcaattgctgttctttagttaatatgatttattagttatgaccatgttgaattgctgttctttttgcttattagttctgacttttgaatttctttttgtgaattgcagttaagacatcatgggaaaggaaaaggttcatatcaacattgtcgtcattggacatgtcgactcaggaaaatcaaccaccactggtcacttgatctacaagctaggaggtattgacaagcgtgtgattgagagatttgagaaggaagcagctgag

SEQ ID NO: 7 - DNA, 1830 bp, synthetic construct
  source 1..1830: mol_type = other DNA
tggaagcccgtgttcgatggtgcctcaaatgcaatagggcatgaaattgttgtcgtcttgatcaaaagaatatgcttcttaaaattgaaacaagatgtttatagcttaagactaatgatttaaagcttcaaaaagaatatgtttcaaaatctaagtaagatcatcattccattccttaggtacatgcttgacttggatccaatctagcaccttgcaccagataaacctcaattcaagtacatccaaaaacacaagtgattgatgtctcaatctcaggacatgaccaacatttgtctgtcagacattccaaacacctgtagtctagcctttatgcttgccatgacacacaagccataagatcatctgcgatctaagcctaactttgttgccatagaataatttcctccaattgactttaggttcctcatctctcaaggccaagtacatgactttcgtggtgaattttctcctatgcatcatttgctcccaacactgcaaattttcaatactctccctcaactgcagaatgactttcattatccaaaagtaactagttttggtcttcacatccatcaaggtgttatgtttaatgtaataggtggaaattcatttcatccataagttatcggattttccatagaggttccaaagcagcttcaacatagtagcctccgttccatttctccaactcaatgattctcatcccaccttggatcttaggtctgcacacattcttccacgcaacagagctcttgctgctaatcttaacacttccaatctacatgaaagacatgcatatactctcaattttatggatcacatgtttagggaaaagaaagcattacatctagtaattagagatgacaaaagtcacactattgattagctggattctctatgcatatctcaacaacttagtactccaatgtttgattcttcctacaatcttatcaacaagattcatgtaatgatgaatgaccaactttttgctagtcaaaggcacccataaatatccgaaaggtaacaaaagtagtcaaatttttgatgcactactttatgatgttatctactccaccaaaataaattttacatttatttggattcatcttaaggtcagagacagaaaacggatgaacgtattcatcataaattcaatagatctaagatccactaacaaacttaccgcaaattatttctattgttatttctatcggatcactaacataataaagtatatttatttaccaagacaagatggaattaaacaaattttagatagcaagtatattagatatttttgggcatttaatttgggccttggtcgaacaagaacccaaactatgctgtcaatttagcaaacctcaacaaaacgacgtcgttccaatcttcaatttcatccttacctaaaacctaactataaaaatactgaaaccctaacatttatcactctctcaaaacttgttctcgattgctcagtctctctctgcggctgaggattccgaacgagtgacatacttcaatttcgtttctgctaaggtaacatccttttaattgataattggggttttctgataattgaatttgttttgttgctctgctaaggattttggttcatctgttagaatggatcttatttggttaagctgattttgaacatgatgctgtttgctgttaactgttaattatcaaagttgggtttttatgatctgaatggtgcttttagctgtttatctgatcttgagcttgttgttgtttactgtttgtctcagtcattattattttatattagatctgacatgtgaagcttctgtgaattgcagttaagacatc

SEQ ID NO: 8 - DNA, 2331 bp, synthetic construct
  source 1..2331: mol_type = other DNA
tggaagcccgtgttcgatggtgcctcaaatgcaatagggcatgaaattgttgtcgtcttgatcaaaagaatatgcttcttaaaattgaaacaagatgtttatagcttaagactaatgatttaaagcttcaaaaagaatatgtttcaaaatctaagtaagatcatcattccattccttaggtacatgcttgacttggatccaatctagcaccttgcaccagataaacctcaattcaagtacatccaaaaacacaagtgattgatgtctcaatctcaggacatgaccaacatttgtctgtcagacattccaaacacctgtagtctagcctttatgcttgccatgacacacaagccataagatcatctgcgatctaagcctaactttgttgccatagaataatttcctccaattgactttaggttcctcatctctcaaggccaagtacatgactttcgtggtgaattttctcctatgcatcatttgctcccaacactgcaaattttcaatactctccctcaactgcagaatgactttcattatccaaaagtaactagttttggtcttcacatccatcaaggtgttatgtttaatgtaataggtggaaattcatttcatccataagttatcggattttccatagaggttccaaagcagcttcaacatagtagcctccgttccatttctccaactcaatgattctcatcccaccttggatcttaggtctgcacacattcttccacgcaacagagctcttcctgctaatcttaacacttccaatctacatgaaagacatgcatatactctcaattttatggatcacatgtttagggaaaagaaagcattacatctagtaattagagatgacaaaagtcacactattgattagctggattctctatgcatatctcaacaacttagtactccaatgtttgattcttcctacaatcttatcaacaagattcatgtaatgatgaatgaccaactttttgctagtcaaaggcacccataaatatccgaaaggtaacaaaagtagtcaaatttttgatgcactactttatgatgttatctactccaccaaaataaattttacatttatttggattcatcttaaggtcagagacagaaaacggtgacacgtggcagtcataaattcaatagatctaagatccactaacaaacttaccgcaaattatttctattgttatttctatcggatcactaacataataaagtatatttatttaccaagacaagatggaattaaacaaattttagatagcaagtatattagatatttttgggcatttaatttgggccttggtcgaacaagaacccaaactatgctgtcaatttagcaaacctcaacagtgacacgtggcagcaatcttcaatttcatccttacctaaaacctaactataaaaatactgaaaccctaacatttatcactctctcaaaacttgttctcgattgctcagtctctctctgcggctgaggattccgaacgagtgacatacttcaatttcgtttctgctaaggtaacatccttcaacttctgtctctctgattcgatctatgctctaagattcttatttgctgtcgtatactatgactctgttagatgattttgttcggttgttttattttatttcattgaatttgtaaactttcttgtgtatgttcggtgatgctttaatcagttgcatgctttacttgtgaaaagttttgatctgagtcctgtttatgatttatcatagttggatctagttgatatgttttaatctcttaaacattgtagactgaatgttgtttcttatgtatttattagtttctctatttaattgataattggggttttctgataattgaatttgttttgttgctctgctaaggattttggttcatctgttagaatggatcttatttggttaagctgattttgaacatgatgctgtttgctgttaactgttaattatcaaagttgggtttttatgatctgaatggtgcttttagctgtttatctgatcttgagcttgttgttgtttactgtttgtctgattttgatagtgatttagcttgttgtttttctgtctttattaatcattgctttgttgttaacatgaattatgagaacttacttttggaacttttttgattttgctgttcagttattattttatacttgtttttgatgttcatctgattttgaacttgatctattgttattttttctgggactaactattattgttattttgtttatgctgttcagtcattattattttatattagatctgacatgtgaagcttctgtgaattgcagttaagacatc

SEQ ID NO: 9 - DNA, 2168 bp, Arachis hypogaea
  source 1..2168: mol_type = genomic DNA
gcttggtacatttccatgaaaccagattatgatcatagtagatgtaataatctattgtagacctcctattatcaaggttcgaagcctaacatggatcatttggattattattaaaagaagactttaatttttttttaataaatatataacaaaattaaagtttaattttgatgcactaacaatataaaatactttacattattatctaaaagtatttatttttgctaatgtagtattacgtaattagatatatataaaagtgtaaaactgttttatactaatagaaaatcaaaattaaatccaacaaagatgttaaaaacttaaactttatatttgttagtgtaaactattatttttaacgacaaaagatttaattgttgtcaatttaaggatataaactaacttgatacaaattaaatatttagttagtttggcaaaactattcaaaatcactaatttgttaggtaattttgtcaaatattttgatagtttgtaatattattctaatttttaataagcattaagttaatttaaatcttttatgattagaattgtctacattcaatttttttcataataaaaaaaataatttactgtattttttatgaaaactgtttcaattaataaattaatacatctaaatagctaatttgggttagtcgagtggttaactcactcgtttgcttaaggcttaatttgataaatttttttgaaaaacataaacacctaagaagctttttaaaattggcttgtgtttatcaaaattaaaaagtctaatataacctcatctattaatcaatatccaaatttagtttttatattaatgtgtattataatatttttaaattttaaaaattattttatcaaacgtattgttattacttgtacatgttgaaattcatttttaatttaatttatcaaacatagatactataatttttaaaaaactatcattctatttttttcttgtcagaaaaaaactaactttcaaaaactagtttttataaattatttttaaaaagtaaaaattttattaaactaagtctaaacatgtatctaaaatttaaattccaccttatatatacaaaaatttatgcatctgaagttttttttttttggtataggaaatgcatctatattcttttttttgggggtcttgaaatgcatctaaattctaaacccttaatatatacctactaatttaaacgaattcagcaaccctaaaacaggcccataaaggaatcgggtcgaacctttaacccgagaggtctcaaacttgtgccgccgctttgtaacttaaccaacgagatcttggcttatcgttggggttcaaatctgcgccagataagcacaacaacggggacacaaacggaatctaattcacaaatttgagggcaattacgtcaaagtaccaaattaggttctgctatatatatccctctaacccttatcactcattctcactttgctcccatacactcttctctcttacttgcggctagggtttttcgcgcatctagctcttccaaggttcgatctctcgttctctcttatttaccttctttgatttgcgctattcttgtttcagtgatgtgatgtgattctgcatgcgatagggaattatcggttttcatatttcatgttcgtatctcacaaatcatgatttttcaattgatttatgtttcgctattttttagtgttatttttgtattgttcagctcttcagttattttgttttaacggattttgatttctgtcttatttatattgaggctttaaatgaattggcttagatttatgaccttgtttaattgaaaacggtagcgaaacacctttgaaaaatcatttatattttacgtttcgattttttcctggttatctctgtatagatctgttgcgtcttctttgaataacatgtttctatcttttaaatattgtatagagaattaagtatgagatcttattttattcgcccgatctgctgaaactttctgtgtctgctttatttctattattgtgtagatatcgtggtcttaaatttgtttgaagttatttcaaagagcataattaggatttgtttaggttttggtttgtttatcatgtgttgaagattgttccgaatacacatctcacggatctattttatttgcagatctttgttcggaatc

SEQ ID NO: 10 - DNA, 2019 bp, Arachis hypogaea
  source 1..2019: mol_type = genomic DNA
gtggcctgcaacggtttatgatcaatctttttcacacgtaaaccgtgccaggtagtaatggtttacatataattttgaaaccgtggttagctccacgaattacataataataaaaaaggacatgcacgtaaccagtttgcaaatgttttaatcaagtaaatctttctctgttttattttatataagtacattgccctaagtttaattcttatattaatgtctattatagtatttttaaattttaaaaactattttatcagacgtatttgttgtgtatttgtacttgttgaaaattatttttatttaatttatcaaatcatagatactataatttttaaacaaaattatcttttaaaattaacttttataaactactttaaaaaagtaaaatttttactaaactaagaatttgtttgggtgagcttttaagaaaatatcttttttcgagttatctttttttaaaagatcttatgaaaaaagtaaaagtaattttatgtttggatatctcatacaaaaagatccttttatctatcaattatgtttgggtataacaatataaaagtacttttatgtttatttattatatgaaaaacatatttttttaagagaaaaatatcttttaaaaaaagatgtaaattacaacttctcaaaaaagatgtttcttttttaatttttctagtacttttatttttacgactagaaatttaccaaatacgctaaaaaataaaaaaaatattttttcattgaaaaaagatcttttttttatcaaaataataacgccagaacaaacgctaaatttaaatatgtgtccagagtttgtatccattttatatatacaaaaacttattgatcatcggtacatcaaataaaattttgatctgcaacaaattattggttgaactatcgaatttaggaatactagaataaaaaattaatgcatctaaactttttctttttttaggtataggaaatgcatctaattttttttttttttaatttggtcttgaaatgcatctaacttgtaaacccctaatatataccgactaatttaaacgaattcagcaaccctaaaacaggcccataaggagtcgggtcgaccctttaaaccgagaggtctcaaacttgtgccgccgcttcgtaacttaactaacgagatcttggctcatcgttggggttcaaatctgcgccagataagcacaacaacggggacacaaacggaatctacttcacaaattcgagggcaattacgtcaaagtaccaaattaggttctactatatatatccctctaacccttatcactcattctcactttgctcccatacactcttctctcttacttgcggctagggtttttcgcgcatctagcccttccaaggttcgatctctcgttctctcttatttaccttctttgatttgcgctattcttgtttcagtgatgtgatgtgattctgcatgcgataggcaattctcggttttcatgtttcatgttttttctcacaaatcatgatttttcaattgatttatatgtttcgctatttttttttgttatttttgtattgtttagcttcagttattttgttttaacggattttgatttctgtcttacttatattgaggctttaaatgaattggcttagatttatgaccttgtttaattgaaaacggtagcgaaacacctttgaaaaatcatttatattttacgtttcgattttttcctggttatctctctatagatctgttgcgttttctttgaataacatgtttctatcttttaaatctatagagaactaattatgagatcttattttattcgcccgatctgatgaaactttctgtgtctgctttatttctattattgtgtagatatcgtggtcttaaatttgtttgaagttatttcaaagagcatattaggatttgtttaggttttttctggttttggtttgtttatcatgtgttgaagattgttccgaatacacatctcacggatctattttatttgcagatctttgttcggaatc

SEQ ID NO: 11 - DNA, 2387 bp, Arachis hypogaea
  source 1..2387: mol_type = genomic DNA
tatatatatatatatatatatatatatatatatatatatatatatatatatatatatatatatatatagaaatatagtgcttaatttttcttagagagaaagataaacaggtccttaaccttttgttccgtaagtatttttgtccttggctattgaaatatatttttaaattcctaatcaaaacttggacggatcagtccctccattcaaatgcctccgtcagacctaacagaaaaatctgacatggctctcgtaatacttgtcacgcgtacacgtgtacacgtgggtcacgtatacgcgtcgccggacagacttccctctcacgcgtacgcgacgcatcgccataaattcaacaaattcttatttttttcatgaattctccattttacatgcttttttctatttctttcaaaccatttttgccttcagggtctatctcatttacactgtaaataaaatacatttacactgtaaatgagataaccacgatgcagctgatatgtcatatctcgtttaaactgtaaacgagatatgattgaaaacgcaaaacgataaatatttttaaaattatttatttccgtaattaatgcttttttgtatttatttaaataaaaaatcttagtttacagatgagttttgatttttttttccctaatgttcctagaaaaaataggattttaagaggttaaagatgagcatagaaggataatttaaaaatttaaagtgtttttaatagaattaataaaaatctacaatgagatatttttatcttttataggtatactattagtttagtttttttatggttaattttaaaacatttaaatctttgatgagtagaaactataatttactcctttgtaaaaataacaaaaatcagttgagtgtttactcttttattttacacaaataaattatatatctataaattaattattgaattaaggtaacaattcaggtgcagtcgattttacgtgaagttgatattgagagccattagataatttaactgattttgactaaatttttatctaaggctctcagatatcaatttcacgtaaagtctacttcacttgaattttcaccttaaattaattattatatatttatatatatttatacatattaatttatatatattattttgttaaataattaactgtgagaataatcaaatctctaataaccaaataatcaaaattatttacagattataatttttttaaaattgcaaatacatttttatgcatgatgactaagtagtggttattttttcttgtatatatatataacgtgacaattttataatactactgtttatatatcttatattttttaaaataaataataactaaaaaaactaaaaaattagtacttttcgtaaaaactaaagaattagctaatatttactcaaatataaaaaatattagtcatgcacataaatgaaaatattaaatggatccaacaaccctaaaaagcagcccaatcggatttgaatcaacccgctaacccgaaaatccgaaatagctaaaaacttgtgttgtgccgccgcattataatgcaacaaacgagacgagttgttggcgcaacgctggggttcgaatcttcgcggataagcacatcaaacggacacaaatggaacgtggtcaataatctgagggcaattccgtcaaagtaaaatatcgaattcagctatataaacactcttaaccctatcaaactcgcacactctgctcccgtacactctccacgctcacttgcggctagggtttttagctcagctccttcttccaggttcgttcctttgtttttctttctctctttccgttcctttgattcgtcgttgttgtgttgtgataatccatgcaatagtttatttttcgatctatgttttcagcaaattatagttttccgatatttcttggagtacttttcttcagtataaatgttttcttcgattattctggttttcttgatttttatgttgaatatttgaattgatttggttcttgtttcgatttatgagcatgtttaattgaaaaatagatccagaattttgaaaaatcgcatgtaattttgaatctggatattctcctttttatctatgtatagatctgggggatttggtttgaatatggttcacaatgatttgctaaaaagggtaattattggttcgtggtaatattcgttcgatctgttgtgacgtatctctttgcttttgtgtcttgaacatttcaaaagcgttaatctctgcatattttgatcaaaacttattatttgatttgcgagtatgtattgtaattttgtatgcgagtaagattgttctgaatgcgaatctcacgggtctattttatttggcagatctttgtttaaggttttaagaaatc

SEQ ID NO: 12 - DNA, 3029 bp, Arachis hypogaea
  source 1..3029: mol_type = genomic DNA
gagagagagagagagagagagagagagagagagagagagagagagagagagagagagagagagagagagagagagagagagagagaaacgaatgaattgtgattaatagttaaaggtgggagtctatatatataattttctccaagatatttagggtgcggagacacctacaccgtaatatacgtatctcgagtactgtgacatttattcgtatccacacgtatttcggatactgtgatcacaattcctattttgaccatatatcgggtgttgtgacccgcatcagtttttgaacgcattttagatgttcagtacccatttttccacttgcatgtattccgactgcactctggatacatcagatttttcatgcgtcatatccatggtttcaatacatgagatacacccatatttgcatttacctccgagatgtggtacattgtttatttaaataattattctgtatttcaataaattatataattatttaatttaaataaaaaatcttaaattattaagaagagtagaaattatccaaattacgaagaatccaatccaacctttaaccaaagcactcacatgtcaaatctttacactttaattaaaggagtgagctcagcagtcagtgatactaaagggagagtgggttgagtgtcaaagtagagccagaatgcaaggagcatggagaaaataagtcaataaggagcgtgggcatgtgacggcgcaatagggtttgatcctgacgcctccgaaattgcaaaaaggcactggatctgtccgacgaactctggtgaagaggcgaagagggaaaggcagttgcgacgtctttcactatcataaccgttttattctttctaatagcagtggtgtttcctgtgggtactaatggcgatgattatttcattggattagagtgtcttaaagtggttccttttaaatttgttatgcaggtttttgctaagtttaacaatagtggattcaacgatgattttagtgtctaatgctgtttttaaatatgtcaatttacttgacttgtcttttgatgagtttgattgatttttttctatcaactcataactccaaagttgagtacatttttctacattttctaattaagagtaacattacatttttttttgtctaactttgaaaaataaattaataatggaattaaaaacaacgcacggcatgagaatgctttttttaactgctttgtttgttcttagcttttttttttttttttttttaccgaaaataggaagactcgaatcacaactttttaaatgagtatgccatttaaattataacttattaatttgttataactcatttgttcttagcttcttgccaacttaaagaatagagtcttgatgaatataaatttttataagaaaagacagttttattttaatatagatagttcacagatgacttttgattttttttttcctaatgttcttagaaaaaataggattttaagaggttaaagatgaccattgaaagataatttaaaaatttaaattgttttttaatagaattaataaaaatctgacaatgaaatatttttatattttgtaggtatactattaatttagttttttatgattaattttaaaacatttaaatctttaatggataaaaactgtaatttattcttttgtaaaaataacaagaatcagttgaatatttactcttttattttacacaaatatattatatatctaaaaattaattattaaattaatcactatatatttatatatattcttttgttaaataattaaccgtcagaataatcaaacctctaataaccaaatattcaaaattatttacatattataatttttttttaaattgcaaatacatttttatacttgatgactaattggtggttattttttttgtatatatatataacgtgacaattttataatactactgtttatatatcttatattttttaaaatgaataataattaaaaaaaactaaaagatcagtacttttagtaaaaactaaagaattagctaatatttactcaaatataaaaaaattagtcatgcacattttattttaaagtaataaataaaaatattaaatggatccaacaaccctaaaaagcagcccaatcggatttgaatcaacccgctaacccgaaaatccgaaatagctaaaaacttgtgttgtgccgccgcattataatgcaacaaacgagacgagttgttggcgcaacgctggggttcgaatcttcgcggataagcacatcaaacggacacaaatggaacgtggtcaatagtctgagggcaattccgtcaatgtaaaatatcgaattcagctatataaacactcttaaccctatcaaactcgcacactctgctcccgtacactctccacgcacacttgcggctagggtttttagctcagctccttcttccaggttcgttcctttgtttttctttctctctttccgttcctttgattcgtcgttgttgtgttgtgataatccatgcaatagtttatttttcgatctattttttcagcaaattatagtttttcgatttttcttggagtactttctttagtataaatgttctcttcgattattctggttttcttgatttttatgttgaatatttgaattgatttggttcttgtttcgatttatgagcatgtttaattgaaacaatagatccagaattttgaaaaatcgtatgtacttttgaatctggatattcgcctttttatctatgtatagatctgggggatttggtttgaatatggttcacaatgatttgctaaaaagggtaattattggttcgtggtgatattcgttcgatctgttgtgacgcatctctttgcttttgtgtcttgaacatttcaaaagcgttaatctctacatattttgatcaaaacttattatttgattttgcgagtatgtattgtaattttatatgtgagtaagattgttctgaatgcgaatctcacgggtctattttatttggcagatctttgtttaaggttttaagaaatc

SEQ ID NO: 13 - DNA, 2141 bp, Cicer arietinum
  source 1..2141: mol_type = genomic DNA
ggatgagtgaggagtaccagggaattagaaatattatttattgcagtcaaaaaaataaataaaatatattttataaaagaaaaatataaaataaaggctcaaactttttttaattaaaaagaacatgaatatttaattggtcttgaaaatttataaaaataataactaataatattataattttttataattaagtgagtgaaaataatctataatattttcacataaaaaacgtttgatctaacgttaaaaaaaatgtagagtatgtaaaaatatatcagaaaaatataacaaaagaaaaaaagagaaatggtggtggaagtaacgtctattatttattgcggaatataaaatatttaaaagtatatttcattaacagaaattctatcaataaaaaaagtttgtggagaaaaaacccctttacctccggaatgaatacacctttgcagtaaaagtgaattcatataacgacaaaatatcttgatggtattattattagttattagttttctatttataaatattttttgaaataaattagttataaattcttcattattaaattttatcgagtctttataaaatttaaaagttatagaataaaaatataaatcatgacctacccaagtcactaatgctgggatgttagaactaaaaaaataatttttaataatgaaaaaaatcttctactgaaataatttatgatattattagtttttcataaaaagaaacctaccatgagaatattttttacttttaaatagagaataaattgtattaaagaaagtattaaaagatacgaaaataatttaatttaatatagagtcaaaatagtaatatgtaaaatcaaagaaggtattaaatcaacttataaagatttgagtttcaataaaaaatcataaaaagattttttttttttttgtcaaaaaaaaaaaagattttatttgaaattagtaattgagccctttatataaaccttgcaaacccttcattctcatcacagtacacacattctcttctcggctagttttaaggctccttcgaggtaaaatcctctctaattttctatctttttgatattcttgttatcgattcataacttttttagcaatgtttagtttaatcatggttttttctatattcttataagattcatggtgtattctatattgatggcttgaatcgttcctttatttagtcttccttgtatcgatcgtggtgtttttttatcttttattttgtattaaactaatgtactctgatacatatttgacatatagttcaaatccttgttattttttgtttttttccccaaaaatcactttttttctaaaaaagaaatcctaaaaaccctaatttgaaaaaaatataccaatgaattactttcagtctatgttctgcattttaaacgaatattttatgtctaatgtatgcttgatcttctggaacatggcttgtgttgctcttttaatattgtgttgtattgttcacatggattgtgctttttgtattgttcacatggattgtgttttttgtattgttcacatggattatgctttttgattttcaatattttaagaatgtgagtatgtcatagaccaaagaaaagaatgtggacatgttaaaatatttgaacctgctgcaatgtttgaataaattctgttaaattctgccgaatgttattgaaaactctacttaattactacaatttcttttgtattttttactaattttatattatcaattaatacaaaaaattggaaaaaaatattcgtgtagaaggtcttcaatgagaccaagtactagccccaaatatgttttgctaacctagtgaaaattaaaaaaagtaagacattttggtatattatattttaagtgaactattttgtatttttatagaaaaagggtaggaaaaaattctgttaatgttcaatcttatgtatgtatttgttgtgtatgcttgatcttgtggaacatggcctgtgttgctcttttaatattgtattgtattgttcacatggaatgtgctttttgattttcataatgttgatttaagacttatatttaatttactaatgtattgtttccttactgtttgtgttcaaattgcagaaattttgtgattttaagctgtagcagta

SEQ ID NO: 14 - DNA, 2177 bp, Cicer arietinum
  source 1..2177: mol_type = genomic DNA
cgttacctgaaattaagttaaaggaccacagatgtaatttagcctatattttataaaagaaaaatataaaataaatgatcaaaacttttttaattaaaaagaacatgaatatttaattggtcttgaaaatttaaaaaaaaattaataatattataattttttataattaagtgagtgaaaataatttataatattttcacataaaaaacgtttgatctaacgttaaaaaaatttagagtgtataaaaatatatgagaaaaatataacaaaagaaaaaaaataatagagaaatggtggtggaagcaagtctattatttattgcggaatataaaatatttaaaagtatattaaattaaaaaaaaattctatcaataaaaaaagtttatggagaaaaaaactcttacttcaggaatgaatacaccttttaggagaaggagtaaaagtgaattcatataatgacaaaatatcttgatggtattactattagttattagttttctatttataaatattttttgaaataaattagttataaattcttcataattaaattttatggagtctttataaaatttaaaagttctagaataaaaatataaatcatgacctgcccaagtcactaatgctgggatgttagaattaaaaaaataatttttaataatgaaaaaaatcttctagtaaaataatttataatactattagtttttcagaaaaagaaacctaccacaagaatatttttgactttaaatagagagtaaattgtacaaaaaaagtattaaaagatacgaaaataatttaatttaatatggaatcaaaatagtgatatgtaaaatcaaagaaggtattaaatcaactaataagatttgagtttcaataaaaaattaaaaatcataaaaagatttttaatttttttttgtaaaaaaaaaagaagattttaattttttttgtcaaaaaaaagattttatttgaaattagtaattgaggcctttatataaaccttgcaatacacactctcttctcgtcacaataaacacattcgcttctcttctttgcggctagttttaaggctccttcaaggtaaaatcctctgatttttttatctttttgatattcttcttattgattcatagtttttttagcaatgtttagtttaatcatggttttttcgatattcttatgacgattcatagtgtattctatattgatggctttaatcattcctttatttagtcttcctcgtggtgtttttttatcttttctttgtattaaaccaatgtactctgatacatatttgacatatagtgcaaatccttatattagacatgttattttttgtttttttccccaaaaatcactttttttctaaaaaagaaatcctaaaaccctaatttgcaacaaatataccaatgcattactttgagtctatgttctgcattttaaacgtatattttatgtctaatgtacgttaatgtatcttagtttttttctctctaaaaaaagtagaatcttaatatatacctaagaaaataatgtggacgtgttaaaatatttgaacgtgctacaatgtttaaacaactcgtatgtatgttaaatttatatttcaataaataaatttgactgcaaaatcggataggtgattgcaaattaggtggaagttgttaatttatttgcaaaatagtaaaataggatggagttgttaaattataggccgaatgttattgaaaactctacttaattactacaatttcttttgtattttttactaattttatattatcaattaatacaaaaaattggaaaaaaatattcgtgtagaaggtcttcaatgagaccaagtactagccccaaatatgttttgctaacctagtgaaaattaaaaaaagtaagacattttggtatattatattttaagtgaactattttgtatttttatagaaaaagggtaggaaaaaattctgttaatgttcaatcttatgtatgtatttgttgtgtatgcttgatcttgtggaacatggcctgtgttgctcttttaatattgtattgtattgttcacatggaatgtgctttttgattttcataatgttgatttaagacttatatttaatttactaatgtattgtttccttactgtttgtgttcaaattgcagaaattttgtgattttaagctgtagcagta

SEQ ID NO: 15 - DNA, 2283 bp, Cicer arietinum
  source 1..2283: mol_type = genomic DNA
ggaaaatggacccacgaccaaaagcaataaaatctaggcatgcaaaaaagtgagcagaagacgttaaaaagtaagttagaaaagacacaacaaaatactattggtcttacttagatcttggaggattattatttcttcacggaataaaaagtcctaatggtttatttgatcaatatataattgatcatgttatcataactttgttatttatcaaaagtccactttacaaatttctcgtgtattaaaaaatatatgcagttgtattaaaaaaaaggtatgtatttaatttttctattttatgcataaatattaaataattgtttaatataattaaatgagtttaattttaatttttcacattttgattttgagacacttagagtgtattaaattagaggggagcaaaatctttttaaaatactttttactacttttcaattttgagaagatttgaaagagagaaacgctagcggactagaaatattatttattacaataaaaaaacaagtataatattttttgtgtatgaatatttaattggtcttagaaataaaaattaaataatttataatattttaagtttttataataaagtgggtgaaaataatttataatatttttacataaaaatcgtttgacgtctaacataaaataaaaaaagtagagtgcataaaaatataagagagaaatataataaaaacaaaaattaaagaaaaatagagaaataatgcgaaagcaacatatattttcttttattatgaaatataaaatatttaaaagtgtattaaattaaaaataaatctaccaataaaaaaagcttgtggggaaagagccccgtcacctcatgaattaatccatctttttggagaagaattgaaagtgaattcatatgattaaaaaagagatgacttataattttaatgttttaagatcttaagtgaaatatggtatcaaaatattttgatagtattaattattagttttttgtttataaatattttttaaaataaactaattataaagtctttattattaaattttatcgagtctttataaaatttaaaaatttaagaataaaattaaaaatcatgacatatccaagccactaatgctgagatattatacctaaagaaataattttttaataaaaaaaagttattacgagaatatttttgattcaaatagagtaaatgataccaaataaagtattaaaagatatgaaaataattcaatttaatatggaatcaaaatagtaatatgtaaaatcaaagaagatattaattcaacttataagatttgagtttcaataaaaaattaaaagtcataaaagatttgatttttttcaaaaatgataaaaagattttatttgaaattaggaattgaggcctttatataaaccctcgcaaacccttcattctgatcacagtacacacattctcttctcttctttgcggctaagtttaacgcagcattctctccttcaaggtataactctctctgatttgatatgttttcaatattctcattatcgattcataggtttttttagctatgtttagtttaatcatgtttttttggatattctcatatcggttcatagtgtattctatatcgatgacttgaatcgttcctttatttagtcttcgttgtattgatctgtattgtttttatattttggattaaacttatgtattttgatgctattaattttttagtatcaattttatcacttagagttttctgtagcgatatacttgatacatattagacacatagcgatatacttttagtatccatgtatgtagtataaagatgttttttttttattttttctccaaaaattactattttctaaaaaagaaatcctaaataccctaatttgaaaaaaatataccaatgaattactatgagtctatgttctgcattttaaacgcattttactatgagtcatagaccaaagaaaagaatgtggacatgttaaaatatttgaacgtgctgcaatgtttaaacaacgtgtattttggatttttttacagaaaaagggttatataggaaaaaattctgttaattttcaatctaatgcttgtatttgttgtgtatgcttgatcttgtggaacatgccctgtgttcctcttttaatattgtattgtatagttgacatggattgtgcattttgattgtcataatgttgatttaagactgatatttaatttacttatgtattgtttccttactgtttgtgttcaaattgctgaaattttgtgattttaagctttagcagta

SEQ ID NO: 16 - DNA, 2228 bp, Cicer arietinum
  source 1..2228: mol_type = genomic DNA
gtgagagtgtgaaatgaagtgtgagttactgagagtgttaagtgagatatgagttattttataaaataacaaatatgaaagagttcgttaaaaaactattagtaaatattattgattattaatgtaattgacgcgtgtgatgcacaaattaatattcattcataaaatatatattataattcacaaagaaaagagaaataacaattttacaaagttgttctaattaaatattagtatatttcaattaccataaacataaagaagtgggataataagagtggtgaacatattattaataagaagatatattagaggatataatttgaaaaaaagttgtattgaaaacttgaagtgtcattcttttttagataagatatttttgcaaatgttacgattattgtaggaatagaggagtatccttctatacaataatttagaactttatattttgtcatataagtataatatatatatatatatatatatatatatatatatattattgaagtattatatttatttatcagttatgtttaatatttatttatttatttataacaattttgatgtaatatctttcaacaaaattttgatataatattgaaaaaaaataccaatgttcaaataatatttatttttcctccgcctctgcattttatcaaaattccaataatacctatttatgtttaaatacgttccataaatcgctaatgttaagtttgaccatatttcataaaactcaatttttaagttttattatacttaatttcttaaattatattatataaaactccaatgaagattttataaaataaatatatattatttactattccaaatttcaaaatttgaatttcacactgtatatatattttaaaaataaataccacaaaacttaaatttattatataatggaagtatagtattaaaaaattataaaaattaaaaaaaaggggttatttttattaatttaataattttaatgtgggaggattagatgtggggatataaaaaaaaaacatccttccttattttattttcttgggcattttgaatttgggccacgatcaagagcccaaaacgatattgcaatattgcaaacctcaatgaaacgatgtcgtatgaaactctaaactaacaactttcttatttaatcatttattttacctaaaaaaacttctacaagcgagaatcccacgcgtgcacctcaattccaagaaaaaccaatagaaaagccacgagagtaactacgacgcacttatcataagtgtgaaacaaaaacgttcaaataagaaagcgagggtatattcggaaattaaaaataaaacgaaggatactttgggaatatgaaaaaatctttatgactataaaaaagttgtaaccctagttttttcattacaactccttacttgttctttcattcttcagtctctctctgcggctgaggattccgaacgagtcttagatttcatttcttctttggtaacaaactttcttatcttctcttttttattcgatctatgttcatttctgatgattttgttcgtttcgtttcatttaaatttgcatgatttgttttgtttttgatttgatgattttgttaggaaaattttgtgagctctttatactctgttacaacatgagatcttgttaataatttttttttgtatgtaattggatctattagaacatgtttttaaattcttttaaatttgattatttgaagttgtttatattcagcgcttttttgtattttttttatgatatgaaatcatgtgtattgttaatgttatgaaattatgtttttttttttaatgttttaatttcttttggttagatctgatttgtttaaactgttttggaatttggtgttgttaacaattatagagcttaggtgtattatttttttgacagttgtttattcaaagttttttcttttacctctttgagtatatatttgtcgagtccatcgtcgatatgattctcatgtaataaaatttttgtataatgttaacaatttgatttatctgtgttttgattactctttgataaatattttttttataaatttgctgttgtaaacaattgatgactatgttagaggctgtttgttattttgtctgctcaattatcatgttgttaaattgctaattaacgtgatttaatttgagcatattttttctgacttttgaaactcttctgaatttctgaattgcagctaagatatc

SEQ ID NO: 17 - DNA, 2241 bp, Cicer arietinum
  source 1..2241: mol_type = genomic DNA
gctcgacaggttattcctccttctcaagatcgttaaatgaaacatccagatttacatacaatatcaatctgttataagaatatatactttaattgtttaaagatttaattgccgttttagtttccttattttcataaattcatgaaaattgatcctcctattttaaaattcgacggttttgattcatcaatttgattttgtaactagtagtgatgatataatatattttaaatgatatgacatatgatacaatgatgtataatgactaatatccatgaaattacaataaaaccctctaaaaatttcatttcaacttttgaaattataaatttttataatttaacggcatatgaataattaagacatctagatggttcaacatcatgagaccacatgttacgtcatttaaaatatgttacatcaatattttttagataaaaaatgtgagcgattgaccaaaagtgttgaatttttaaatgaagggactaaatatgtgaattgatgaaaatagtagatccaaaattgtaataatatatttttaaaattttatgaatataaaattcaattgaattatatatattccttccatacactattataaatcaaaataatatttttcacggttattaaaaaaattaactaagtgcaattaatttttcttaatttcatatatataagtgaaatttaaaaacttgtcattttttaaagtcactcatcaactattcagattaaatgtttttgaattaaatgaataatatattaaaaataataataataatattaataataataacaataataataataatattaattaatgattaaataatacttaatatttatctataattattttttttccgcttataagtataaatttaaagaagtacactcttaaacttaaattttatataattatttggacctatggtattaaaaaattaaaaatttaggggtattttgattaatttaattatctaaaggtgggaggatagatgtaataaacaaaaatatatccttccttcttgtttttttgggcatttggtttgggccacagtccattgagagtccaatatatatgtctatactttaaactaacaaccttcctatttcatccttttgcctaaaaaactcacgaaagcattttctacacgcgaatccacgctgcgtaacaacactatctagaaagatctaaaaaaaatcagggagagtaaatagcacgcaccgtcttctaagtgttgaaacaaaaagttcaaataagcaaagtgagggtatgttcggaaaactaaaagacattgaaggacattttgggaatatgaaaaagatatttgtgactataaaatgttgtaaccctagcttgtttgttgtcatcactctttacttattcattcatccttcagtctctctgcggctgaggattccgaacgcgtcttcgtttttcttaggtaacataatttcttatcttctctcttttattcgatctatgtttatttctgatgattttgttcggttcttatgatttcaatacgcatgttttgttttgatttttatttaatgattttcttaggaaactatactttcgattttattttgtaaactttgacatttttaagttttttttaactgattgttttatatatacatagatctgatgattttgtgtctgtatcatgttttttatctgatgattttgtgtctgcatcatgttttttgtgttgttcgttttgctttgatatatcacgagatcttgttaatatttatttttttattaacgggatctaacaaatttgttcttaatatcttaatatatttttatttttatttttttctgtgcttgtttgtatatctatgtatttgttatttatctatgtctgttaatgtttttattcacctgtttgattttaacttatttggttaacctgtttttgaattggatatgttaataacattcatagctgaatttgattctgtttttgataatatatctattccatgtttttctggcaattattgtgtaattgaatttagtttgacgttggcaatttgaatcacctgtttttttttttttaattttatttgattaagcttgcaatgaattataatatatgatgttaacaatttgatgagtttacttggatgttatttttatgttgtctctgttcatttattatattattttgttaattgacgttttctgacttttgaaacttttgtgaatttctgaatttcagctaagatatc

SEQ ID NO: 18 - DNA, 2177 bp, Cicer arietinum
  source 1..2177: mol_type = genomic DNA
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttattattattattattattattattattattattattattattattattattattattattattattattattattattattattattattattaaggtgtgtcataatccactaatgtggagagtcaataaaggtcgctcaaagagcaaatgcattataacagaaatggacactacagaaagaataccaagaaaatgcggattatgtcggctctctggtcatattatgaaacattgtacaaacgctgttggcacatctactcaaattgaatgtattttttaaaattttaatgtattttttttaaatttataatataatttaatcattttaattaaataattataattataataatttttaattataataatttttaattataattataataattatttattataatactaataattaaaatataaaatttatttataatttattaatgtattttattttaatttattattaatatttttattttttaaataattaaattaattttaatttattatcctaattaataacaaatgttaaaaaaaaaaggtcagagttagatcattccatggggaatcgacctcctattacgtcttcaaaattttcctggttgcttcctggagaagtaacatcgtactttaaaaataatagaacattcgcttaaataattttcaaaataggatattttgaaaattaaaaaaataaaagggatattattatcaaaaaagttcttaatacacgttatgtcagatataaaatatatttaatttttaagagactaataaatatgattaaatgtttttggaggagtaaaaaatttatagtttaaaagggcaaaaaaagaaaagaccccaaaaccaaaaaattcataaattttggtaagtttagaggccaacttcttcaacaacaaaaaacatcgaaaatttattttcaagaaatgtttttttgattttttaaagatttacaatttttatattttctctttgaaagttttttaatttaaaaatcaaaatcaaaattttcaagaaaaataattttttgattttgctcacaaaatatttcaagaaaaacataatattttagattttcttaaataaaattttcgatattaatattagaataagttataaatcataaaaagatttacttttattttttatcaatattaaaagatttgatttattttcttgtcaatattaaaatatttgatttgatattaacaatccagatctttatatataaacccttctaactctacgttctctacccactgcactctttctctttctggggttttaactcagccttctccttcaaggtaaacatgatcctattttctttcttatcgattttcttctcttcgattcttaggtttttagcattagttcagatatgcgatgtgatttcataggatccttagattttaggaattggtactgaatatgtagtttttatttgatatgaatgcaacaatatttgagtatctatcttttaattgttagcttataaacataatcgacataaacaagtgatattcggattatactgtgtttgcttgatattttgaaagacatggcgtcttcctctttcaatattatattgatgacttggattgtgtctttattttgttagcatgtttctgatattagtattattttgttttaaatttttgttctgggtcatacttgtgtattctgaagctatcaatttttgggtatcaatcttttcaccaagagttttatgttgtggtacttaatacacgtcagacataaaatatatttaatttttcagagactaataaatatgataaagatgttctttttgaattgtttacttgtatttattttgtgtctgcttgaaacatgccgtgtcttgcctttttgatgttgtattgtatagttgacatgggttgtgcatttttatttgtgagaatgctgttgttgctagtactttatttatctatttttaatttgaaaactggtgttttatttttcttatgtattgttttctcactttctgtgttgaaattgcagttattttgtgattttatgctcaaggacta

SEQ ID NO: 19 - DNA, 2242 bp, Lupinus albus
  source 1..2242: mol_type = genomic DNA
ggaggagatttagagagagggagagagaaatagtgatatgtccatgtcttctagatgaataggaataaaggagaaaagaaatataggtataggaaagaaggtacttaaattgtccatttgaatcatgagataataagtattcaaagaccaaagtgcttctagaatgcatgggacatacacaaacacttcaataattttattttatttaatggaagttgtcgaggaagtttccacatgtctttgctaatgtttaattaataattaggtagggtctaagatccaaataagaattatgtttgatattatgaaaaaaagtactaaatcaatatatagtgtaagtcacattatgttatatctaaggtgctaggatttttaaaattttaaatgttaattattcaatggttaccatgcccatataagatttattcatgacttttctttatcaccaaagtgcttcataaaattttatgtgttttataactagttattatgaactattttttcttcttaattagcacttgagttttatgatttaataaaattgattattttataaatataatttaaattactaataagccaacaggtaaatcgctttgataagactcacaacaatagtattgcaacacctagtcaaaattaatatttagagaaaattgttgattgagaagaaaatgataaataaatttatacttaaaagtgtgtataatattaatatatatgtatagaataaagtaataataatataatatttgcaaaaactaatttcactaactcttaaagtttaggaggcaaatacaaagaaatttttttttatggtgactactcataaatcgcataaattttagaggaacacatcctatgtccaaaaaagtttgtcacttcatatcttacttgttgtgattggtcagagaaatatttaattattaaattttgaaattaaatatacgaaacaatagacataatctcaaaattatgggcgtgcttatagagttttccatttattttattgtattatattatttttgaactatcaaaagtattcttcacattatgagtaatgaactaaccatttttagggtgcatgattgtccatcacaataatcgttactttgaggattcaatcctttaacattataggagtacattagacaagatcaatgacttattagtatatattatattatttaccattaattaatcgacaagatatcaggaatcaacaatttaattgagaatttaatatatatgaattgtgagcgcaaaatgtttaatatattattacatttatctagaatgaagtggataataattaatatataaatagtaaaaaaaaccttagtcctataatgaaagccttggtttcatttatttgtatattatttagtatttaaaaattgaaacgtggataagtgggtggagtgagccctaataataaaaagaacatgaatccaatgagtcgggtcgacctatttacccgtcccacgccctaacttgtgccgccataagacatagagcgaagtgccacgtaagcagaacaaggacacatctggaattaaaatgctaaaaatcaacttgatttttatgctatataaacgccactgtcctcgttttctcttcctttttcgccgctagggttttagcgcagcttcttattattcaaaggtattctttctctacatcccttttactattcttattatcttcttattccattttctctttaactcttcatacttgtatcatttaatttattaactcctttgtgtttaattcatccattacggtcgatgattattcacgttttatttcattttttcactttgttatttcttgttttaattaaattactgtatctaagtattttaaaaactgcatatcctatttggaatcatgagtttctgtaagtcattcacgtatggaattgctattagttttttggtttcttgttattttttaatatcgttggtgtagatgaaaatgtttgtatgagtgttgttatcataatgattttcttttatcttattcatacaacatgtggtatatgatgtgcttgtatgttttattgattaggatttggttttttgagttgtcaaccatgttattgaggttgttattttgatcatcttattttattctcttccataattggatttgatattttcttgcattttcttttgtttttgatagctttctcatggttcattttctgcaggtctatctgtttcgtttttaaagcaaa

SEQ ID NO: 20 - DNA, 2629 bp, Lupinus albus
  source 1..2629: mol_type = genomic DNA
cgtgctacctcctgtcccaatactttgatagaatctaaatcctctatccatttattttatcatcattttttgtcgtttctttggcacctattttcatcaccttctataggttatagtgtcttaattgaactacttccatgtattaaacaatttgggccaaggttatggatctgcatgggctgatatgacataaaaaatctgttgattagtccaaaatgttccagaatttttctaattaacatagggaactatccatgaccattatgatattaattaataagtattatattttgattttccacaaaacttaaatacatgtaattttaaattgtttgcacttgtatttgtatattagtatgttagatttaaaaaatctagtaggagacatatgattttttgttgttgcaatttttattattttgtaattgtatggtggatatccgagaaattcaccttatctatactaggcactagcgtgttgactggaattgtcatgcccgtcaatctactttcctatcgtttgtaacatgtgctttatatgtaataaatgaattgtgttaaagtgttagatatattataattgcagttgaatatattatgatttgcattgagttatatgatttttagtagaaattaaaatacaacgagagcaaaatagaaaatcaataattacataatggtaaataattattttagattttgactttattaatagtagatataaatttagagcgaacctaagcaataaatttattgttcactcccaaaccacataaataatagtataatggatggatatttgataccatattaaaaaaaaaaaatttcatttaaagtcttttataattatttatcatgtatgatttatcctttttacttccaatgccagttggacacgcacaacttccacttaggtcttaagcgataagcgaagtgggggtatatgagtaaaatccattgtaaacaaagggtattttaggaaacgaaaaaggtgcaacttcgctataaatataagtgaaccctaaaattttcatttcactctgcctctattatctctatttctcagtctttgtcttgcggctgaggattctcatccatcaagcgttcttcgattgttctttaggtataatttcttccttttcttctctcttagtttcgatctatgtttcttgtttcctttcatatctgattatctgtttacgttatcgatttccatgttttgaatctgagtttgtttgatctacgttttttttttacatttttatgcttagttttatgtttatgagcttgatttctcgagtttttgttcattccggcttcgattcttctggctgtatacacagatctaatgttttgaaagtttaaaagttggaaaagtttgaggatgaagatgtttatgatattttctgtgtttgattatggatctgtgttgttataatcgataccgtgtgtttgatttagagttttatttttttaatgttaatttataatttctcttgtttactttaaaaaaattctgccgtgtgttgatatataacttatcgaaatatttcttttcaacattttgtttagaatttttaatttgatttcaaacttttttatttagttcttacctgttttctcttttccttctgtgataatatgtgcctgtgttaagatggaatagcttttgctcttatagatttgatttatctgcttgataacctaaatttgtttttatttatagttgttgaatgctgttactgtaaatttgcctagttttaatcctttttgttttattcctgcctgaataatattgtttcaaattatttatttcaaatccgttgttgaagattaaagatttgattttgtaatgaaatggttttgattttgtagtttaatgaaacatatttgattttgatttgtagtttaatgaaacatgtttgattttgtaatgaaagatttgattttgaaagaaacatatttgattttgtaatgaagcatatttgattttgtaatgaagcagatttgattttgtaatgaatctgatttgtttttgtaatgaagcagatttgtttttgtagtgaaacagatttgtttttgtaatgaatctgatttgtttttgtagtgaaacagatttgtttttgtagtgaaacagatttgtttttgtagtgaaacagatttgtttttgtagtgaaacagatttgtttttgtagtgaaacagatttgtttttgtagtgaaacagatttgattttgtagtgaaacatatttgattttgtagtgaaacatatttgattttgtagtgaaacatatttgattttgtagtgaaacatatttgattttgtaatgaaacatatttgattttgtaatgaaacatatttgattttgtaatgaaacagatttgattttgtagtttaatgaaacaatgtggttatagatttttgcatgaaataattttaattttgtggatttgatttatctgccttgttaaacaaattgtcttaaactattttgtcgttttctgttcttgtaaaattttgttcacaggattctctgattttccattttggtattgtatatatgattgttctataatttaatgtgtctttggtcctgctgatctttttcttcatttcagcttaagatatc

SEQ ID NO: 21 - DNA, 2111 bp, Lupinus albus
  source 1..2111: mol_type = genomic DNA
gggtacctgcaggacgtgtatcatgatacatattcccacccatgagctatttttgcggatacctattgtgtgtattttgacgagtacccacaagtcccgatctttttaccatccctagtccatctataagcctcccatgcatgcttctaatgtctatctcccatatcttgtcctagcattttgtatggataaaatatggacccctaatttaattatacaaaatttgaatagcaaataatttaattaagtcatatttttacaatatgacttaattaaatatggaaacacagtatacttttttgggattcaactattagttaatcaaagtgtcacttatgtggggccaaaatgaaatttaagcttgagccatatgggtgtaatttactccaaataaaacttatttatttaaaataactatagataagggatgcaataaattttattagaatttaaaaaataaaatattgtttcctattttatattttaaacttcattcttatttgtcaatagttgttttctatattttatttggcataactataaatattctatgcactataaataaggaactaatgttaattattttagatatgtatgattgtatatcctaataagtgttattatagaaaatattattgataatctactaaaacattatatcactctattaatatattgttatttgtcattgctttccaatataagaatattattgaaaaataaacatttaataatatatttaaaatttattaataaataatatttttaaacataattttattcctatagaaagaaattgaagatgttagtctattcatttttttccagaaaaacttaagtcgttaataattattttaaatgaaagaaattataatatataataagatgatacattgattatcctcatggtagaataaggtattagcgaagagggggtatataaggaaatccaaacaaaaaacaagggtatttccggaaacacaaaagtagttccttcagtataaaaagggttgaaccctaagttttcaattccactctgtttcttatctcaaaacctcagtctttgtcttgcggctgaagattctgaaccttcttcgattcttcttctttaggtatcactttttcatttttctctttctattagcttcgatttatgtctcgctttattttcatatctgattttgtatctttgatattctatgtttttcaattttttagtttgtttagtaggtgttttatcttgtttattattagatttttgtttagatgttttgattcctgattttttaatattttcagtttggaatatctctcttgtaatcgtagatctacggtttttgtgttgaaaattgaaatatttaatcatagtgtttgtggagttatgtctttatgatgttttaaatgtttaatttttgatctttaaggttagaaatttgttatttaactttcgagtcattatttatataattaagctttttcttttgttgtgtttatgttatgtgttctttgatatttgtttcattagtgagatatgtcagatgttttggtgtcacgttaagtttgttggaagaatattgttcttagcaaatttgttttaaatatttcatgaagattttttaaatttaatactaatttatctagttcatgattattactttttttgctgcaataccattttgtgcctatgttgggatgttattatttttatgttagagatttattttctgcttgtttagcaaaatttgtatgccttaattgatttttgttttgtattgttgtgtttctctgtcaatttgactagctgggtctatgtttttttgttgtctggttgaaaatattgttttcaatcctttttaagattttaaaattcagtttatgcttcagtgcttaatcgttaaatattttgcttaatttaaaataatatgggtgtatgttattatggtaatacatttgactttataataagattttattacatgcctgttaaataatatttaaaatgttctgttactgtagatttgattagtagggaactaagatcatgtgtttttaattattacatgtgaattttctaactcattgatctttaatcaataccagcctaagacatc

SEQ ID NO: 22 - DNA, 2470 bp, Lupinus albus
  source 1..2470: mol_type = genomic DNA
gtggagtatttcacgcactcccttaatattaaaagttttctgaaagatacaaattttattttaaattacagttttcttattaatattcaaaaatttagaagtaaatatagagtgaaaattaaaataatagagcgagttactttgcatttaattacaccaaataatagtaaagaataaaaatattagatgaatgaatataaccatctaaatattacaaatcaaaattgtatgacgtctattaatataaaatcaacaatctatttaatttatacatagataatagacgtaacttgtaattctattttagagtatttgaagcactctcttattaaagtttatttaaaattgtatttttgttaagacttttccaaaaaaattagaagtcaaaaacttattcttatattaaactttaaaatatgccaacaattctgccctttaaaatactagcatgttttattttaaaatttaaataggtttattgaagacctacaattaaattaaaattttgtatgtgttcctttatattcaaatttcacatacaataattttagttacaattaaaataatataatataaggtattattaatatatataaaaagatcatgatagaggcttatatataatctatttctacctccaactaattacctaatttgttataatttccaaaacgtacagaaaatcttgattttttgttatcgtcagcgcgtgtgaaacaaattcaccgaaagatcaacaacgtgaaacaaacgctacatctcacgcgagtgggacagtaaacaacaacaaaataaagagttatctgaatttgaataaaattatacaaactatccctccgttttttacacctacaaaaaaaactttctgatttacatacatacattacataaactacccctgcatcattgcatgtgaaataagtgtgatttttatataaaaatggatgataaatgtaatgaagttaaacaaaatatgatatattgtagagatatattaaaatagtatacaagacaatacatttttattattttttcattacaaaatgcataaataattcttaatagatattatttaaatcactcttatattatattataacgaacaaaaaataatgaaaatatgttgactcatataccatttggacatgtttccacaatatatatattttgtttaacttaattacatttatcaatttttcttgtttaaaagatcacatttattgctcatgcaagagtggtctgtgtaatgtatgtttataaattcggggttttttataggtattaaaattggagggatttgtataatttcattaaaactcagggaaatttagtgcaatccgcagatagaggatatgcgagggaatctaatataataagaagggtattttcgtaacaaaaaaaagttgatacctctatataaaaacgtcgaaaccctaaaacgtttagtttcttctatcactcagtctctgtatttgcggctgaggattccgaacttcaattctctattgtatgtataatcctcaattcgattatatctcttttttgatttcagattcatcgtttatttaactgtttaattgtcatgtgtgattgaatttgatcgattttttttattttattttggattttgttttaatttattagtcggaaatgatttctagttcatcgtttatttaagtgtttgattattgtgtatgattgaatttgatcgatttgtttgactgtggattcttttttatttttgattgattagttggaagcgattttggatttcattgtcgtttttggctttaatttttgttgctgaatgcatatatctgatgcttttatgttgaaattatagccttgtatgtttaaatttatggatatgttaaaactattatggagtatatgctgttcaatgattttagtattttttagttaattcagttatatttttttattatcagattgtcatattatttgcctgaatttggtgctttgttgatataattcggagtatttttgtcttttaagtttgataatctgctttgtatatattatgatcattgaattttattttgtgttgatgtagagtatatgtacgtgtcttctcttgcatagattttgatcatttgctataactctgttccatttcttatttttgtttgtttgatgctttataatctgagacaaacatgtataaaaaatttcacacttttagggaagtatttaaaattgaatttggtactacgtttccttaatgctgattttgtgtttcttcttgtttattaaagacttttactgaaactcaacgtgttataattgaatctttttaacattgagggattttattttaacattggtgtccctgtgtaatttaatttaaatatgctacattggtttctatgtgcttttatccttgttttttaaaaaatgtaatgttttctgacttgcaaactttttgaattttgatccagtttaagaaatc

SEQ ID NO: 23 - DNA, 2174 bp, Lupinus albus
  source 1..2174: mol_type = genomic DNA
ggttgcagcggacatcactactccattgttgttattggtcatatcattcttaattttaagaacatgaatagtttatacttacttcaaatttttatttattttggtaatacaataaattgaaattgaaatcaaattgtttcattttctacaatttttctggtatcatatttcgatttcttattttaattttgttagaatattcaaatttgaaattatcatttattttcattatgtgtttgaatatgctaatacacaagtaatacttgtgttattaacaatgtgaatgtacgggtaacatgtatgttttaactaagctaatgcatatgtaacatgtgtgctcttagcaatgagaatgtacggataacatttgagttttaaaccatgtgaatgcatggttcacatgtgtgttcttagcgatacaaatgcacggagaccatgtattttttactatacgaatgtacatgtaacatatatttttttaacaatatgaatactcatataatattcgtgttcttaatcatatgataaaaaagtattctctcgtgcgtatacacgggtgagtgaagatcttctataccacaatttctctatctctctataccaagccaatgaaagcatgtcaactaatagtagttattgtttcttgatagatgataaaaaaattattaacataattttattagttgatatgctttcattgatttgatatagaaagatagagaaataatagcataaatgcacagataacttgttaattttaataatgttgaagattcttttcaaagataacaatgatgaagaatagaaaattcttatccgtttctttggtggaaaaaaacgttatagcttatttttttaacttgtcacacaagtaagcgtgttatacaagtaaaacagactagagtcaacaactgaaaatacatttaaaatttagggtttgtttggcgagaatggatttacttactacaacttatcatcttatctatcaattttacataaatctaatgatttagatatatttactaattcaaattattaaatttctgactaaaataagatgtagagtaaattttttaaagatgtcgtccactaaccaataggaaattaataatgaaatattcttctaatctataggttattttttaataatgttatatccttctagtaatactcattgtcacaaaatgatatttattaatacatttaacatatgtcatgattagtacgagaatttagtaggacaatatatcattttcctacttttacataaacccttataaataaaacacaataaccctattgttctctattcacttctttttcactatcactatcgcggtgagggtttagcgcagcttcttcttctaggttcgttcctccattttcttcttctctgatgcgatgttttatgcttcgattgttctattttccaatgaaaataactcatttctatgatgcgatgttttatgcttcattgctctgttgtttttaattcataatatcatttgtcatgatcatcattgttctcttatttgtgacttttctcttttctttcttggtgttattttctttattcataatctattgttttttcaattgatctcatgttattttagcaatgttcagcactgttcactttattttgtggtttcgtaggtgtttacttttgttgatgttaatttagttttgatttttgtaacagtatgagttaattttggtttggcgttgggattctattaattaagcattgttatattaatccacgttgtaggttggaatctacttgtgaaaaatcgtgcatgtttgattttaggatttgttattgtgtgcactgtttgttaaaccgatagaaaatatggatgaacatatatgtaagttgtgatgaagagtgcttgtgctgaatatatgattttctatggacatctctgtctgtgatgtgttttgacttttgatggagtagtttgattagtgaatccggttcaaatgtttattatttcaataatctgaatttttctctgattctgaaagtttgatgttgatagtgattggttaatgcagtgtaaattgtgtgtttggtttttgttatgtatttgtgtgctgtgttaaaggtggtagctattgatagaatttgtgtggttaattttggcagatccttgttttgaaccttagtcggg

SEQ ID NO: 24 - DNA, 2120 bp, Lupinus albus
  source 1..2120: mol_type = genomic DNA
cctggtcgccgattatgatgtacttgaagagatagtcgtaagacatggtggcgaaacaaacgacgtcgtgtgaagaggaaacacaaaagtgagtgtggtgagttcaagaatgaagacttaacggaaaagaaagaaaggaaatatgttgtcatataaaagatttagatttatttagagtttaaattattagattttcttataatgattgaataaaatatgaataaatttttatcaattattcttgtagtcaattggctaataaatataacatttgaaattctattttggttcacttttgttttataaataaatccaaaatccaactcaatctgagttgttgaataaaaaaaacaaatcaaactgatataaaatagcaatggaaaattggaaactgattcaaaatacttctaaaatgtatagttgttatctcttcaaactaataagattatgatgtgttttattttttaaaaatatttttataaaaaataattaaataaaatatatgtggtgattttattggataaatgaaataacttattttctaatataagaaaatactttatttagttagatataggtattggactctacactcaattacaatttacaaacaatgataatataaatccaattgtgtcataagatattatttgagaggaatatatatatatgtcaaccgcatataaattttatatactataattatattatctataattcatatttaattttgatttaccttttaaaaataataattaaaataaatgatggataatataataattatgtataaatatttatgttgttattatggagtatatgagtattattatacttattattttaaacatgtgtctatacaattttttaaaccacatgtgttaattgagtgccacggggacacaacttggttcccttaagcgataagcgaactcggaaatccaaagggtattttcggaaacaaaaaaagtatcctcttcagtataaatatcacttaaccctaacaatttttatttcactttgcttttcttcattcgtattctttagtctgtcttgcggctaagaattctcaacgtgcttttcaatcttcgattcttctttaggtataaaacatctttcgttttcttcgatctctgtttttttttgttttcttttcatttttgtttcttctttactctgtttattattcgcattctattaatctttgattttgttctgtgtttttttttctttttttatgattaggtttgtttataatcatggattctcgtgttttttgttgtttcgggtttgaatatgcgttttgtattcgcggatctaatggtgttgtgttgaaaattgaatcagtttgaggtgttatttgtttatgatgttttgaatgtttgaatttggaactttgtttttataaacgatgctctgttaagattctgagttttttccaatggaaattttgttgaggttgtcttgtttactttgaaggttttttgtttgtgttgatttatgctattatttggtatactaatggattgtttttatttcttagaagctttaggttgtttttcatccatgatatatgatgttctgatattatgtaacgtttgcttaatctcatttattggatctataaattacgaaaatatggaatcgattgtgttgaacattttttatttcaattcaacacttgtttattgagtgcttaattcttttcttgtttgattatttgaaaacaatttgggttgatactgtatttttggatgtgataacttttgatgttatagattttatttatctgttctgaacattttttaatttcaatttgacatttgtttactcttttacatttctgttttattcactgtaaatttgaatattttgaatctcttattttttattttagtgctttgtgagtaaagtcgtatccaaaggatttgtttagtgcttagttatttatgctttcacttaattaaaaaccatatggccctatgtttggaatattccgttattgtcaattaagttagtaagaatatctctttttattgtggtcctgcatttcattatttttgttatttaatgtgagttctgatttgctgatcttttaatctattccagcttaagacatc

SEQ ID NO: 25 - DNA, 2205 bp, Lupinus albus
  source 1..2205: mol_type = genomic DNA
ggtgatgatacgacaccacacctttacttatttacgcaaatattgttgatatgacacctttaacatcgtttatttgagcaagtgctgttaaatatgttgtatcactatttttgacataacttttgaatcactaatcagaattggacatattatatatgtatttggggtagaaaatttcattttatccaaattttctcattgcattagaaagatacaccctgaaattcaaaaaaaaaaacttttttactatttctttcttattttataagttttatagtgaatgtgttttgataatgttagtgtcactgaaaagtcacacatttaacagcggtttttagagagcattgttaaaggtcatacatttttgttttagtacgcgtgaatctttgtagatttaaaaacaaaacctcactcacagtaatacatatatcattcatctcttacttttcttctcttagttcctctcttcaagtaagttgttagcttttgttctctcttactccatgatattatgtttcaagaaagtcatgcattagatgcacaactcctgtgacctattgaatctcgtgcagtacatgcacgattcctaatagggagtcacattaccaatccacgacaatgacaaactcatggacctcatccacgagatttctaaaaactacaccaaaatcgtaaataaagtttccactacagtaaagttggcggtaatttttccggtaaaaaattgagggtgatctatgtaatttcattaaaactcagaggtgtcagtctaattaactctaaaatttattacattttattatatttaaatagtataatataaaatttaatttataaaaaaaactagtgttcgtagggaaatatatctggtgtcccacaaaaatttattactatatgtgtcattatttgttattggttgataaaaatgtatttaaattaaaaattaaataatgaaatatttttatgatttatatatgaaatgatacattttttgggacaaaaatattcactataaactgctgagagaatatcatatacacgctaatccaaggcgcgtgtgaaaaaattggcctagagcagtacaaaggtcaagaaagataatgatacactacatcgcatgcgagtgggatagtttatctaattatgaaagaaatctgagggcagtttcggaactaaaaagttaatgcttcgatataaatttcttaaaccctaaacattctttctcttttcactatccctcaatctctgcattgcggcttagtgctccaaacttcattcctcttcgaggtataatcctctctttgttctatcttttgctttttattcactttctaagttatttcctgattcattttgtgttgaaaatgatgaaagattcatccttttttattgtttaattattgttgtggttgattttgattggttagttctttctggtgtttagatttgcatttgtgatattgggttcttttggttttgtaatttattctccggaatgggtttttgattgttttgttatttgttttggttttggtttttgttgttgcatgtatagatctgatcattttttgtttagattttagcttatatattatgaatttaattctggatctgttgttattatggattctatattgttcaatgattttaattttgtatttatgtatttgtggtttggttaatagattgttataatctggtaatctggattatttttcttgttagagattttgatttgatgatatggttttcttttaatgttttttcttttattttttaatgttttatggttttctgaaactgaatttgtgaaccaatttattctgtgcttgtcaaacaatgtgtttggctcccttgtttagtgattctattttatttaatttcaacatgctacatcgttttttacgcactttttccatgtttgaagtttaacttgatagtaagttttccttaatccgtgtttctcgtgtatatactcttttggtggaattcaacatgttttagtgtgatcttcttaatgtttactaatttattttaagtgcttgtcattcataatgtgttttactcccttgttttgtgattttatttaattttaacttcaagatgctgcattcattcgttgtactttttcccctgtttgattaacttttattattacttttggacttttgcagacttttgtattttgatccagtttaagctatc

SEQ ID NO: 26 - DNA, 2034 bp, Lotus japonicus
  source 1..2034: mol_type = genomic DNA
ggagagcacaaacgtcccagttttttttaagcgcagaaaatgcgagcacctcacgtgcgaaattataaaaggcccctttaagaaaaaatctgctagcggaggattacctcccaaggataaaatacttagtgctaaaaagagagagccaaaaaaggatattcgcttggttaataagaaagataataaagaaataataaatagaaaaaatacatagatcctagccttgaacattgatctttattgcttgagcttctatgattcatgtattgttgcttttgcgtcagttgtcttatttctctaacaaaaataaaggccatttttgtttctcttcaacaaataagtgcttgagctattgtcattctgtttctacttgccgatcatacgtatagaactaacttccataattaggatacattttgggaaaataaaacttctcataataaaatctcatcgataaagaaaaattgcagtctcaatttcactacaagatttgcgaaagtttcattttgtaaagcaatgaaaaaagctattgagttaactgaacatgtgaaatgcctggggagatgaaatgttctcatttttaagggtattttgggaaacaaatccagtggagacggggtgagggacaataggtcgtgggtataagaaactcccccgattctcccgtataaccaggtcttgtgttcggcgaacaatcatgtgctgcgaaaagtcggacttgatgcaacaattttttttttaccatttcgagtgatatcaagatgacattgtgcaaatttattaatttttaataatgtattttatttttctttgacaaataagaaagtaggggaggtcctgcggaaaaatagctcgacgcaaagaaagaggtgaaacagcagtagccacaaatacgacaattcgattttatatacacctagtttggtcggtaaaaaaaagcccacaggcatgaaatcaccaacaaaaaaaaaacaaagggtattcttggaactcaaatgttgttatatggtataaaatgaccaaaccctacatctaactctcactctgctcttaattttgctatcgctcagtttctgcggctgagaattccgaacatcgtttgaatctgaggtataatcttctctttgattcgtgtctattcatttctatgctttctgattctgttcatcgtatgctgattgatgatgtttttttatctgtttaatttgttttttttttcccatcttttgctgtgtctattcgaatccgattccacgttgttggtggatgcttgtgtatggattcggtttttgtgattcattcttagatgtgggatctcgattacttttcttttcggttttgaatcttgttgctgcatgaacagatctacttgtgaaccgttgaaatttcatccctattggctgatgtatgttttgactggaaaattctgcatctgtgagattttgaaatattatttgctgtttgagttttaattttgttgtatatctggttcaatcaaaccaatttcttgtctattatgttgggattgcagttagacacgcctggaattttgttcttgttgttgtccattgatgttgtatgggaatcttctgcctcgatgagtatcgataattgtatattttataatctaactatgaatggtttgtggggacatcaggacatgtgttttttctgtagagattttgaccctgtcttattctttggtaacatgattgtgttatgttagatctgtgattataatcttgtatgaagtatatgtttatatttttgatgcacagaaatagttaagtactgaggtatgttattgcttgtggataacaacaccctgggtgaaactcatgttttatcaagtcctgtttaatgttaactgagttgatttatctgtttggaaaataatgtgtcttgctcctcttgttatggattttgttttattttgagcctgctagattttcattctatttaactgagccattttttttgttgttttttctgacgtgaaaaccttttggaatatgttctgcagttcaattagtc

SEQ ID NO: 27 - DNA, 2184 bp, Lotus japonicus
  source 1..2184: mol_type = genomic DNA
gtgccaagagtaccgtgagcttgaaaaacaaggatttaacatgaagatcaatgtgtatctcatattaatatcatcttttttcttgaggataattaactaatgtttggaagatagatagatactaataattaatatcatcttttttttgttgaacttaattgattaataaatagggtggcatggaaatgttgagcaacagacaaggggagtagccatgacttcaaccgctccaacagtggaacagggcatgacccgttgaaagacttttttacatggctagtgcaattttcaaaacaaattaccaaactagtgcaacttttgttttaattacaaaaatagtgtaaatcgcctctttattttttattttttttatgaaatcgccactagcaatggcgacactataatttttttttgactttgtttaatagtgacggaaaagtgtccgtcacaaattatgtcgctaaaattttgccgcagtagtattcacgcttcaatttccgtcgctaaattctttttcaatattcactaggatttcaagatgaaatttacttcgtcgcaaacagaattccgtcgctagacattttgcgacggatgctatctctcttcacattcggtcactgataccttttatattatatattaggattttgtgacagatacaattcgttcactaacaaagaaagtcaagaaaaaaaaattatagggtgtatttcataaaaaacaaaagaaaaaaaggtgatatcgccaatgagattggcgatttacactaaacatgtaaataaaaaaaaagttgctccattttggtaataaacctttcaaattacaccatctaggtaaaaaagtctcgttgaaatgaggatctgtgtgcatgcatgtgatgaagttcttaattcatacaagtttgtggtgttagatctaaagcatgacaatatatacaaataaataaagtagtttctgtcttttctttttcttttcatttcatttcgtatattcatgtcatgtgttcttatggtgaatatgacattacgatcatatgtacctagaattctcaaaatgtgatgctagttctaacttcaacaattctatatatatatgagaattctgtaacgagtaaaagcagactccttatcatgaattacagtataataatttaaggaataaggatataagttgtttgaaaaaaagatataagtttatttagaaatatatgtatgatctatttaagtatttcaagtattcatttcccgatgtaccaaaaaaaattatctcaagtacattaggttgcttttttcttcataattttgtgtccaacttatctaaatttacactactttaatttctagttctttgaacttattatctttatataggtttctaagcactcaagattatgattctgatacccttgaaaattcaacatataatttatcttatttctatctctaccagcaaaaacacacacttcatcaaaatttcagaaaggcattaggtgcaaggttaaggaagcttgatcatcagtggaagcttgaaaaggtacaaaattgctacaagattaaaaagggcaccccatcgggacagagattaccctctccaaaatcacctgtgccataacaaaaggatttacggccacaacaatccccaacctctatttttgtgacatgtgatgagataataaaggatgtcgctgatactgcagcctacagagttgctgacgccgagaacaaatcatatttggccgctgaggcaatcagggaggtggagagaatctcaaggctggctgaagatactgattcaacgttaaagcttgtgaaagagatttatgaaaaatgttcacatggtgaaattgtccttttggcttaagtcggtgatcaatgtgagcagatattagatcctgcatataaattcatcaatccggtgaatattatttgagtagctctctatttttacacctctccaactgtattgtactggttagtctttcttgtgaagtcatgtttctttgaggctggtacaccttccttggtctctagatattatccatggaagctgtctttcatttgagattgccagtttgccacaaagaaaaatgtgttctttgatgtatacttccatatggctgctgatattagaatttttattggtataggtaagtaggagcaagattccctctgttgctgtaataag

SEQ ID NO: 28 - DNA, 2182 bp, Lotus japonicus
  source 1..2182: mol_type = genomic DNA
cttcttcggttccctcatgggttatttctaaggtatcccacatttctttgactaatttgcgatttgacactctaaagaactcatccatacctagggctaaagttagaatgtttttagcctttaaatcatattgaacttttattttgtcatcatcattccaagagttcgtaggcttaggttgtttaacaccatacatcaacacaaggtatagaaggactttcttcaacagcattccaaatatttaagtcttgagattcaataaatattttcattcttatgttccaaaaatcatactgttcccggtgaaactaggttaatgttagaaccttcactaaaaaatggtttagagaaggccatttatgtgttttacaaggatctagaaataatttacctaaccaaactttggtaccaatttcgaacattgagatagcaacctaagaagggaacgtgaattaggtttgtaattaagtatccaaattaaaaacttacaaaccaaaaattagagtaacatgagcataaaccacaaatacaaaatgaaaaatagaaattgaaatgctaaaaagttaagagttaagagaaaagaagatcactaacagatgtatcctggtccaccttcaccaactgaaggctacgtccccctttctgattcggggatttttccattatataattaatgtccagtacaagtctcacaccaagactctcaagaacatctattaacctatagaagaacaccacttctttcttagaaatcactcaccaaagcagaaaatatcacccaaggcaaaagaaagctcactgtgaaaagttgtaagagaatgagacttttgttgctctaaggaggccgatgactcttatattctcttttattttgtgtgattgttgtagacttttatttttttgatgttgtttcactttgttttgtttgtcgttctagcaatttagttgcggactttctgaccagaacgcctctacctatgctaatttagtttgggtgaggaggttccattagccgcaactgatttggtttggtttctaacgatgtattgacttcaatgcctagttcttcttgattgaatgaagctcatttcaataaaaaaaaataagaactcatatcaatccaatacatgtcggtggtaaaaagtgattgttcaaactttagattgagagttcaattcttaagaaccaaatttttaccagatacttagtcataccataatcaattgacattacgaaagcaatttaattctatggaacacaatgaagttgaaataagcgagcggggcataactggaaatctgaaagagaaccaagcgtattctaggaaataaaagattatctccgtcaatataaaactgtatatcctaaccctagaagattttattatcactctgctagttagtttcctctttcgctcagtctctgtctctgcggctgaggattctgaacaagcaaccttcgatcctcctcaggtatcatctaccatccataattgtctcttccgttctatctacgtttgtgtcttcgttgattcagtgttgtgcttagtttgttttaaatgttcgaaaatgctttttcaatcggatttgtgtttggaattcttgtggattttgcaaactgatcattcttgctgtattcagagatctggtggttctgtgttgaaatcagccagctttgtttgacatgttagaagctggggatgttttgattgtttactgagtctgttatgcttagggaattttgttcatgtgcattgatttgatctgaaggttgttctttttgttagaggaatttttctgatatttgttggttgataaattgcttgaattttactccatgctgttcttcattagctaaatgatgttaagtcttcttaaaattttcatgatataaatatcaatatcatttctggatcaagtagacgcgtttttcttcagtgctcatgaattcttttttgtatctaattggaaacattatggatgggactctgattgctttgtggttgcagttttgatttatcttgttagatgaattgcacttggctttactggtttgaaagcttgttatcaattactaatctgctagttagattctgtcatgttattttgtttgggtttagttattttataattaacttcatttctgagtattgaacctcttgttaattgcagtttgagacgtc

SEQ ID NO: 29 - DNA, 2208 bp, Lotus japonicus
  source 1..2208: mol_type = genomic DNA
ccctcatacttcgggcgtagtgtcttgtgggcttgtggactaggcaagaccggcccatgatatgacctgcatagagttagagctcgtcgtgactataggacgactagacgaggtctccttagggcgaacaactagatcttcagtgagtatagggatacttgtctcttatagagagaataggccacacccatgagacccaagagcccaatcaagataacattaggattttggaccatctcactataaaataggagatcactcattgtaaaatggatccattcattattacatgatattattatttctcttttctaagtgtgttaaggtttggtatcaaggtctctattccatgtccttcttagatctagaacatatagagccaatcaaattgctttaatttaaatatttaaaaatcaaagtttaatatttttcgagaggatttaaaatataaaaaattgatttattgtaatttaataattgtgtaaaatttacactatacgtgtatagtaattttagttaaaaaatttgataaaaatatttttagttaaaaaattggcataatagattaaaaaataaaaaaatattaaaaaggaaaagatgaatcgggtgacccgtttaataaaaaattgtcaaactaccacgtccttgtctgcagcatcagaacggcgttcccaaatcccaaccctagcgtcacgacattctcgtaccctaataaaaacagattttcacgctctgataagcgcgtgaatgctactgcaatctcttttgtgatccttgaaatacacgcgcctatcacgagaacgcttgtttactagccgacaaacaagcagaaaaggattcaaatggaaacacagtaaaatatcaagggtatttatgtaaacaagaaaaaagagctgcagcgtataaatagctcctattagcctcgtttatatcacttctcttcacgcttcactctttttactctccttcgttttctattctccaatttcggctagggtttaacctcagctactaggtttgatcattttatcaatgctggttctttttttcttcaattcgtgtgtgttctgttgaattattctgcattgtatgtatagtttttgttttcatctctcatcatccgcaatttgtatgctcttttattgatcatttaatgatttttcttgtgtgtgcatgctatttgcttcaacggttatgatatatgtgttgttaaatttgggatttctttttttgtgtctaatgtgtttgcgctttttttcattgtgtaactgatttgattttggttttgagggtttatgtgctttgttatttttttcgaatccttgttctagatcttttgtaatttttgtttttgatttagtgttgtttttgttaactgaagaacagatctgtttaaacattggaactgaccaattgcagttgaggattttactctgtgttatattggttagttccagattctggaattgatttaaggcccttttaactcagtggtagagtaacgccatgtaaggtgtaagtcatcggttcaaatccgataaggggctttttaatttttgattttataaaacccggcaggggagttgtatttcaattttgttacatgctgtatttttttaatctgctggaatttgcaatcatgttatgtttgtattttgatcatattataaatcattggtacaaataggggcttttgatttttttatttcataacatgctgtttttttaaatctgctggaatatgcaatcatgttttgtttgtattttgttcagccttcagagtatgtttatttgtcagcttgataatttgttgggtatttaggtcatcagatatttttaggctcttaattttatattaatggtggagtccttaatctttgttgaacttgctgtattttttaatctgctggaatatgcaatcatgttatgtttgtattttgttcagagtatgtgtatttatatcaatggaggagtccttaatctttgttgaacttgctgtattttttattctgctgtaatatgttatttttgtattttgttcagagtatgtttatttcaacttgataatatgttgggtatttatgttatcagataattttaggttcttaatttttgttaatggtgaagtccttgttgcttgttgaatttgtttttatcagtatatctggataattggatagccttctcactgtttcttttgtagcagtg

SEQ ID NO: 30 - DNA, 2312 bp, Lotus japonicus
  source 1..2312: mol_type = genomic DNA
gggtggtgatgatggatggtactattttgtgttgtcctacaattttgaggatgatgtgtttggtgaggttatgctgccggagacattcgcttgtgccagctccgatatgcctaatgttacggttattggaggcggcgatgggaaagctctcactgtgtaccatgtggatggtgtcatgcagtgctcttgtgatatctgggtcatgaaggagtatggtgttgtggggtcttggaataaggtttttagtttcaatttggtgggttttgatatggaactaacctcttttgggatacttgtgaccggcatgacagctccgctgacgcctttgtgcgtgaggaatggtggagaagtattggtgctcatggatgaggcggggagtggatgtctgtattcggtaaatcttgaaaggaatgaaattacagactgtcaaattggaggagaggagttcacatggtatctttattctggttactatgctgagagcttggttttgctagacaaagcacgcggtttggtctcttattgaagcagaggtaactgtgacctatcctgaaatatgttcacaatagctcaattggatgctcaatttgttttaggcaaatgctagtgccctaaaggtactcattaagtactaaatgttgtatctcaattgtgagcaggacctttctgttgtatatttattgtacaatggtacaatttacttcacctggttgctttctgaatgggaggcatacctctccttaggttgtttgagtgcataaacaaatggatggtatgcgtatatgtagtgtaattgaagtctttgtctctgtttcttgcttatgatgttgtttatgttttgaattccaaataccaactagttttcttgctaatacaatagctgtattctgtctgcttcaagtttctagtaagaacttctgtaacaaattattaattattagagcttgtgtggcgccaaaacttcttcctgtatttttgagatgttccctaaaagaatacatcattctgtgcaccctgcttatgatcgtagtttttgcttccatagtctgacagaaagctttaaccagattctgttggaagtttgttgtcccgagtaatatgatattctctactgaaacattgttatcctatttctagtttttatctgcctggtctcttgttaagattttcatttctaatataaatcaaacttctgggaggttaaggaaataggtatcctgataacagatgactcaatctgattgcaatcaaagcacaattttcaggagatatttatagattgtcttcccagaccactagtaaaatgaaagtcgctcatctctgtttcaagcttgaaattttgaaaccttggaatgtggaacagacatgtatgtatgtacttgtgtgtttattaagtgagatggagggttctttaggtaagtatggatttgtttatgcaaccatgaggctggtcttaagtaagctaaccaatttgatggggaggacgcatggtgtctttgggcaccataatggctttcagcgtaagagcttagttttactaaagaaatcaagtggtttggtatcttattgaagaggaggtatacttaggatggatcatatattaggatgctcatgtgtgtgtgtgggatttgctatgatactccgactagattttgtgaaggagtagcattcaacctcttttttgttatgacaaaaacaccatctctttgttcgtgattaatttattcatgtaatttcattttaattaatattttgaaatcaataatttctctcaactcgttacccactctctttcacctaatctatcaccttcttcctcatctccgaatctttaatgctaaaatgactgtcttgtagtgtaaactctagactgaacccgaggaatgttacatacataaataaggttggaatttcttgatttgcacttggaattagatgtattgaacttcaggtggaaacaacggaaactaggactaggaggatatttattggcaatatttgaatatttattaattgttggaagaagattaattattggagctaaattcttggactccaaggcagagactagtatgaaccagcttctttgaaactgtcatgttgaattcttgcactccaaggcagagactagtatgaagtgaacctcttcgaaaatgtcataccgttggagttgaattcttgaaaatatgtcatattataaaataactcctactacgaactcttctccacactcattctctctcaatcagatatatatatatatatatatatatatatatatatcttttttgttttaagactcaggcagagag

SEQ ID NO: 31 - DNA, 2621 bp, Medicago truncatula
  source 1..2621: mol_type = genomic DNA
gccttcatacgtttgaccaacaccgttaatttcatataatttgattactcaattattttaattactctccatcaatttcatataatttgaatttgaatgtaattcctctagcattcaatgtgatattattctaccaaaaatataaaatgtaggagtaatattgtgaatagaaatatttatccgaggtgtcttatatgtttaacctaaaggtatcctttgattagagcaaacacttgaattaacatatttagtctagcattaatgatgattgagaaaataatgatactgatatataaaccacctgtattatatagaaagagattcaaatttttatattgtagattagtaccataataaagttgaccaaacaatacttcgatattgcaaattaaatattaattttattatccccgtgagcttagctcagttgatagggatattgcatattatatgcaggagccggggttcgaacctcggacactccacttttccacaattaaattgtgtgagctctagccattagactacttgaccgaaaaaaaaaatatgaattttattttatagtaatttcaattgaatttaaatttgagaaataaaaattatgtttgtatttgattgatatatgcatatgacaccattaattttaattaatgagtgagatactacaaatttataaattttacccaacaaacccaaaacaatcaatctatcaattttctttcctacaattgctagtgtagcaaattctttataaacacgacacaaagtggagcagaattgtgttatgcatttgcactttgatagcattcctcaaatgaaagatgagtttattaaaccatgcgacatttgtttttcgaaaaacaaaacaaaattggtttgagagttatgggtttggaactgtgatggtgatataaataaataatggaaagagaattctatgaaaatgaattgcaatgaaatgaggaatatacatgaggttatgttttgaaatgacttcattgttggtttaatattatttgatttgattttttctttgatttttttgtacacaattcatttccttttgtttgtaggtgatttaatgaaattaaaaagtacataattgtctgatttaatgtgatattaaaagttgtaattaggcctaaaaacatgtcatgtgtactattttatagagaatgtttgaattgtttatattgtagatgtagattatagatacacatatattccttaaaaaaaaaaaatacacatatattgaagaatattgtatcttccattaaagcactctcttgatcattttttgaggtcaaatccacttggttaagtaacctaaagtatagacagaaccctaaaacgctccttttctacaattgtcgtgccgcccaaggaaataacgctagtccacatgattcattggaattaaattaggtcaaaagagggcaattccgtcaaaatcgaaaaaaccgcactaatatataaacccttcactttgtaaccctgtttactcttactccactctcattctcttccttgctgctagggttttagcgcatccttcaaaggtaattcaatatctcctttttcgtttctaattcacatatccatttcgccgattcatttctgattacgcagaatcgttaaaaattagtttctttttgttttcatttctgattatggtatgataatcatgatgaaatttatgtgcctgtttaattgttattctaaattttgataacgtgttaatcgtttatgattttggtattcgattatgctgtgataatctggtgaaatttatgtgcttgtttaattgttattctgaatttagatattgtgatgatcgtttatgattttgaatcggtagttttttatttgatatgaaaatttttgggatcgttattttgacctaaagttttctgttgtgctgattgatacatatatgaatatacatgtatataatgttattttagcatcgtttatgcttgttactcttattttattttaattttgttgttgaattctgttttctatttagtctccttcaaacttgaacgttagaaattttgttgttgctcttattttgtactcatgtatggtcttcatgtgttgatttggctcatcgtttgtgattttaacttgaaggaccaagggttttgtattttcaaacgtgggtttcttttatatgttgttattttgtttgattattctgctgaaatcttcattgctttgattttggtatgaatgttatctgttgtgatgaagatgtatacagaaatgcgtgtgtgatgaagatgttctttttgaattgctttatgaatgtttgtgtttagaatattatcttactgaatgtattatgctctttaaacttgaattgttgacctggatatcatgaagtatgtatcagaatgttgctgttgttgctcttatttaataaattaatttattgtttgaaactctgaatggctgatcatttgtgattttaagacacatgtctttatccttgtgtcttgatcaattgatagtttaccgttaatttccttactttttctttcttttgaagtatatacttatgttgttttctttgttgaatttgcagaactttttgtggttttaaacttttaaggaca

SEQ ID NO: 32 - DNA, 2456 bp, Medicago truncatula
  source 1..2456: mol_type = genomic DNA
cgtcctaccagtggcggagttaggattattataaagtctgggcaaaaaaaaaaattacaacacattttttttctctgcaatttttgagatgtaggtctttttcactctctgggtttgggataaattgcatttgcagcaaataatcaacattcagagggttttacttaagaaattgcaagcaaatgataaaaaaatctaaaaaaattgtccaacaaaaaatatacataagaaatttgtaggagtttatgtaagaaattaagaggaatttacataaaattttgcataagctatgggctagagcacgagcaactgcctaggctaggtgggccttggctccacccatgtcatacacatcccttaagtcaagactggtataacacaatcgctctaagagttgtgagtacatgtcacacaactcttccctgaacacgatgtattgattacaaggtaccatacccctatactttctctcggaattttccaaaatttgtcccccctctggcaaatgaaccataatttgatgaaccctatcttcaaaacatgaagttatacacataaggtgtgtttatcgcaaccttcgtgccacatcgagtattcaacagacacataagctttttgtttttctaacaaaatatggttaaaaaaacaacaatgtaaatgtttacaaacttaaatgactaaattgttattaaaaaaaaacttggggaaatgttaaaaaacgcatttaggacacttgctaaagacttattttttaaaaaacctgtcataaaaagtggtgtattcaatcttactcaactttttgaaagttcgaaatttgttatttccaataccaaattttcatttttccttttcttaactaacatccttggatctcactcgttagcgtgacttagactcttacaacattatcttgtcatacacctctagctttaatgacatcacaattagcatgacctaaaaacttaccggactaacttttttcgaaatgaattgacattgtatgtcggacattatgacctatttgacattatatgtctattaattaataattttttgaagaagaaaccttcatgtttggttttgcccaatatttcacttgggccacactcaaagaaaagcccaaaaaaacaatgtcacagacattacaaacctaaatgaaacgatgtcgtcacaataccttaacctaaccttctttcttattccatccttacctaaaaaactcacaaaagcatattccatccgccactctccaacgttcacattagggtattttcggaaatccaaaattcagcaaggacacttttggaacataaaaatatctgagaccctataaaaacattgtaaccctaggttttttcattctcactcatttctttcactctcaaatatcactccgtctctctctgcggctgagagttcccaaccctagatttcgattcagctgaggtaacaacaatctccgattcttgtttcattgattcgatctatgtttctttgttgttcaattacatgaattaaatgaatctgtttcataattttgttcggttaatttgcatgttttgattttgtttattgtttgagaaatttaatcatgaatgaatcgatttatgattgtaatattggatattgaatttgtttgagctaaattttgtatgagaaattttaattttgaatttaatctgattctgtttattgtttgagaaattttaattatgaatcgatatgattttgtaatatttgattaattctagctaatttaaccgattgttaatctgaaattatagatctgtgtattttgttgttgaaatgatttaattttgtttgaaaagttatgagtttttgatgtttttattgtttaattatggatctgttaatacagtactgtgattttaatttggtttcgtttatttaattaaaatggtcttcaaatttgagctgtgtttgtttgttgatgtgaaaatgtaatttttttgtatctaataacatgagtctggtttatcatgtttcataattggatctagattatgtatgttgaaagtctgttctgaattttgattaacaagtacttttttgttattctgatttattgattacttttggggtatgactcttgtgttgttggatttggtttgatgagatcttgtgtggttaagttgattttaaatttcatgttaatattgataagctgagttggattattgcttgtctgtttcacttattatcatttagcttattacacaatgaattagttgtttggttaagcatgatttgtttattacaatgaaataatttacgaattagcatgataatgacttttgaaactgtcaaatgcggttcaattattttagtgaacatgattcagaagttttgaaactttgcatggattttcatttgttatggttttttactgttgatttctgacttttgtagtttttatgaattgcagttaagacaac

SEQ ID NO: 33 - DNA, 2462 bp, Medicago truncatula
  source 1..2462: mol_type = genomic DNA
tgaggggtctttgagtaatccaatgagaatattgaataaccattatgctaattggccaacctgtagatccgacctaatttgacaggtttgggtacaactttcagtatgttgttccccacctctcaaaattttgtgtgcgagttttacaactttgagcatgtagttccccacctctcaacattgtgtgtgtacaagttttacaactaaactctctcatttggtcaaaaagttcaatgattaaaattttactaaatgaactatgctcatgggaacaacaactaaagtctttccaaaagtaaatgaaaggatatttttatgtagatgcaggatatgcatgagtacccctcttctcacaatattttttgctcattcccaacacaaaatagcctagcatgagttaactcacgcatgagaatactcctgcgtgagttaattcacactaggctattttgttggaagtgagaaaaaaatgttgtgggaagaacagtactcgatatacattaccaagtgaattatgctcttcacccaacacttttcgctcgcaccctgcaacgttgcctaaattatccatgcgcaagttaagtcacgctgcgtgtcttaacttgcgcacgttgtatattaaaggaactttgttgaatgcttgaatgaatgatgaaaaatgtttgcttgaatgaatgtttgaaagaaggattgaaaaaatagggaaaatagaggtttctgcctcttctgtttgtatgcacagactgtgtgtgagttaactcacgcatgtgctaatggttgcgtgagttaactcacacagtgttttacacttgcgcgctgtaagttgcgcaacgttagttaactaacacataattttacacttgcgtgagttaactagcgcaagggtaaatttagaacggctgcagggcgcgagcaaaatgtactggatgaagagcataattcttgccaagtatagatgatgtatttcatttgggccacactctagggaaagcccaacccatgtgatcaatttcacaaacctaaaacaaatccaagggtactttcggaatatgaaactatctaggtcactataaaattcttacaaccctaatttcttcattctctcacacttcacttgttcctttatcactcagtctctctctgcggctgaggatccgaacgagtaacaaccttacatttcgtttcagctgaggtatcatcctttgattcctttctctttgattcgatctatctttatttgttgttcaataatatgaattaaatgatgctttttaatgattttgttcggttcttttgtttaattttcatgtttttattttgtttcaagtttgaatttttttttattgatgttcgtttatgtggttaaaagggtcttcgtttttattgttggtttgagtttttgttgtgattatttgtcaatccttcaattagtgtcatgttgttctttgatatgttgtaatatgatttattgtttttgtttcatgatttcatgatgttgttaattaattggatctaattgatatgatgttagtcttatatgtatgttttaacatgatttattgttttttgattaatgattttatgacgatttttgaaattggatctagttgatatgtttaataacaacgttgtatgttgtatttgttttgttgttaatgatttggttaaactgtttgattgtatctttatttgttaagctgattttgaacatgatgatttggttaatctgtttgattagatcttaactggtaaagctgatttttttttcttgaataaactggttaagctgattttgaacttgatgatttggttaagctgattttgaacttgatgatttggttaatctgtttgtttagatcttaattggttaagctgattttaagattgattttgtcaattattaacgtattgttgaactattgatgtgtatcaacatcaagttcaaaatcagcttaaccatgtaagatctaataaatcagtaccaaatatattggttatgatttgttgcttttctattagattggtttcatcgagcgtagggctttgttaactggatcttatttgttgaactgatttagttagggttttagcttgatgtgttaaacttctgagcttgatgttgttaggttttgagattgatatgttaaacttattttgggattgatgttgttagcttttgagcttgatgttgttaaccattgatgaattattctgctcaattaaggtctttgttttgattagatcttattttcttggatctgatttgttgtttgcttaatggtttgatcaattattattatttgggttattaacctgaattatttttttattaacatgagttctggcttttgaaacttttttccgatcgatgattagttagtttcttgctcattaacttatttatttttaaactgatatatgttctgacttgagtaattttgtggattgcagtttaagacatc

SEQ ID NO: 34 - DNA, 2136 bp, Medicago truncatula
  source 1..2136: mol_type = genomic DNA
cccgtgctcgcacagtatcctcgtgggtatccactatgcgggtatccgtgggtattgaggtatccatgggtattcatgggtacccatggttttttgaatttattttttttgtacaaataataattttttaattatcaaaaatcttcaacttttaaacatccatacataatccattccaaatttcacaaatcatacataatccatagaaaattcatgatttttacataattcaggatatgttttatgatgtgaataaagtggaaacgatgaacaaaataagatgaattaaaaaaattaaaattaaaaataggttattaatgatgaatcttaggtgagcggtaaaatttccttctataaggtaacaactttaagtaataagattccctaaaaaaactttaagtaataagataagataaaataagatatataaggatattttagtcatttatttaatttatcatcgggtatgggtactcgtgggtatggatacagtcaaatccgtgtccgttttcatataataatggatagtcaaatacccatttattatacccgtggatactcatttacctatctgtcccgtgtccgtaatggattttatccacgggtatccgtggccatgagtttttttgccacccctaatttgacaattcaataaattcattagtaattattatttttgactctaaaatcattactagtttctgatcagacaatttttaccttacgactgaatttcatattactagaatcatgaagtcctaaaactcaccgtagggaagggaagttaattattattttaatattaattaatattaacgaggtgaagtgaaaatcttagaaaattctgtctaaaaaaaaaaactcaacatattatttatttatttatttatttggagggatattatttatgagtaatgatacatatacatccttaggtgacaatatattcctggacacccacacaaaaatctgacacgtaatcatatggaccccatacaagcacactttctttttcatctcatcttctctcttccaaatatatagggtgtacaagggtgtatgttaataaaggatgtttatataacattttcctaatttatttattgttgaagacaattttaaaatgtttgttttgaaattaatttagtacaattccatgactttataaaccctctaaccctcccttgcaataaaactctaccgctttgcaaccaacagaaaaaccaaaaccctggcgttcctctctgctcttcttccttcaaggtaacatttcgatttcgtttcttatcttcgattcatagcttattcatatagttgattttacttctgttaatacattacataatttgatcatagttttttttttgttttgtttctggttttactttttgttttgaagctatatcctgtgtttatgtacacgttaaatcgcaattctgaatccaaaattatgtaatgatcgttttttgtttatgaatccgtaatttttgtttgttgtgatgacattcttttttttcatttagggttttgtatgttcatatttgatgcatatatacttaacatacatgaacatgtgaatatcaatattctttttggagtaaaccaccaaattcgtttcaaagtagtcgccgactctgtcaaattttagtctattagtctgaaaacgtgttagggactaaattgatagtaagtggttaaacatagagttttaattgataacagcttgttaaaatataggaacgtaactgatagtagatggttaaatatagggaccaacatgatagtttaatagagtcagggactaatttggtggtttattcttcgttttgaattttatactttgtgtttgttttcgtatcatgttgtgtttgatctatctgctgaaacactcggtgtctatgctactggatattgaattattgacttgaattttgtattttttttgtcctgcgttggttatagtattaatttttttttccttgggcacatacatggattgttttctttgttcgatcggtagaactttttgttgattgtagttttttttttttcttttttcctttgggatcatacttatgcattgttctctttgttgaattggcagatcttttcgtggttttaagcttttaaggacaa

SEQ ID NO: 35 - DNA, 1975 bp, Phaseolus vulgaris
  source 1..1975: mol_type = genomic DNA
cacaccctaaatctctgctatagcccttaaacaactattaatgataaattaggtcaattaatacggaaaatgtgtgaaatcttaataaatgtaaaggaaacttatatacatttattaatatcaaaatatatggaaagtcaaaacaataaataaagtgtcttgaaattgtattattatgaattattatattattcaagatattattcgttcaaacttgaaattatattactgagtagaattattgaaatatttaaggttatattgtgttatttaggaatatttaatttgttttgacctgatttatacactgtatattaaatataaagaaagaaatattgttagtttaaaacagtaataattttttttgctaaattgaaacttgattttaaaatacctaaaaagtgaaatctcaattgtaagatatataatataatatatataatatataatatatatatatatatatatatatataatataatataaaaatcaagtctaaataaaaaaatctctgttttgttgagtttttaattttaacatttacaatttattttatacatatttatttatgccatctcattataacgctaaatattttgaaataaaaaataaaaaatatttaattttaaaatataacaagtgataatgtttgtgaaaataaaatcaatgcaaaaataggataaaatatctaaaacgactacgtaagtagagaaagatttgatcaacgttgacaatgaggaatccaaaggttgaaacgatgtcgttcaaatgtcttaactaagtcacctagctgaaacgatgtcgttatagtaacgcagccttcctgtttggtccttgccagctggactatgcgaacgaacaaaaacccgatatccctgctaacgtaaagcgcgtgaaggttagccacgcaagatacgcgagaaagcagaatcgtcaattacacgcgccttctacgcgagtggtacacagtaactttcaaataaggaaacaggggcataatcggaaacctagtaaaaccctaagggtattatcggaaacgaaaagtagctcccacaatataaaacaattggaaccctagttcattatcactccttgcttcctcgctctatttttgcggctgaggattctgaaccagcgaccttcgtttctctctaaggtagcagcctctgctcttttgtctctctgattccgttcttctctgtttcttgtttacgatgatgtttcttcggatttttttaaaagaaattctttttttagtactaaatgtgcggttctttgtttgttgtttcttttatgtggattatcttcgcttttctaccgattaatcggaagaggctctcgtttcggtgttgaccctcgttgtgttcacagatctattttttgtgtgtcataattatgcggttttgtttattttttttcataggttaagaagctttgaatgatttattgttgttctgctaagactgcactattgtggctggtgttcgtttacatgcaaattttcgatgtcttggatgatttttatttatggtgtctgaagttttcatcccacgtaacaatacgaagcttttttttttcctatttgaatttatttggattctgcagatgattttttggttacccttttgtaaatcttttgtagcatgaagttaatttttttaattaattaaaagtgtttttgtttgtgttcaatagaaaacgtttggtatgaatacgtcgtctgggagggtttttttattacagatttgttatgttatgtaggattgtgctttgcttacctgtcttttcaattgattttcttatttcttatttttttattagctagagatgttcgagttgaagcatattcttaatgatttgtttttttttcctgcttggttgaagacgtttcaatgctgtgatgaatctgcttttgtagttgctggttgtgagttctgacttgtgtttctttgaattattttcagtttaactcgtc

SEQ ID NO: 36 - DNA, 3033 bp, Phaseolus vulgaris
  source 1..3033: mol_type = genomic DNA
tatatatatatatatatatatatatattttaaagaaaaaaacattaaattatttttaaagtgaagtccaaataaaaataaagattgatatttatgatatttttcaacatattattcaaaggaaataaaattattgttaatgaatatctttattaattcagttataataataataataatcaatttatataaaatatatcatttataattcttttaattttaaaaaaatatttattattttcttattcttatcaagattaaaaaaaaaatagattgttaagtattatgatttatttgattttcttaagaaaaataaaaattgaattacaacgtgaaataaataaaaagttaaaaaaatcacaatgaaaatggaagaagaaaaaacagaaagttgtttggtaattgttttggaaaaatagttttcaaaacgaaactaaactataatatcattcttaaaatttatttataaaaaggaaactataatatcattcttaaaatttatttataaaaaggaatcatattgatagatgagtataatatatttgtatttattaaaataaattataaatatacactcttaatataatatatatgaataataatattttatttaattttaaatattaatttttctacaatttgttgtttatttagattattgacaaatcacaagtcttatataagatctaaattttataaatttataattaaaaatcatgcaatttaactattcatattaaaaaaaccaaattttataaaaaaataaattatattaatgagatatttatagaattttacttaaatctctactacataaaataataaaaatataatatattgcatgatatttagtaactgtaaaaaaaaatataaatattacatgatatttaataactgtaaacaaataaaaatataatatattgcatgatatttagtaatagtaaactaaataataaaaaattatgacatggaaacttttataaaaaaagaaatactataatttttaagatagacaatgataggaaaaaaaattaaaaagtttcaacgactacacatttaaaatttaaaattaataattgctttctacaaaatgagaatatattagtttacaaaagtaaaatagttaccaaatttattgaaaaaacacattttttttattcaaaattatatttttctttttttagttttatattaataataacaaatcaaaattagtgatttatcattattgagaataaaaaagttgttttaatataattatatttattatgttataaatttaaatttgtttaatactttttttattataattaaatagaaaaaataaaaattaaattacattactttttaaagttcttttaccaactcgatgaaattattatatttgacaaaattatttttaatatttgatgcaaatttatttttttgcaaaattaattaattaaagaaaaatacattaaaataaagataatacataaatataaaaatgatatgatgtaaaactaatctattataccatacttaaaagatttgttttggaaactgcactaaactacgtcgttttggtgggcgcataattaaaccctaaccagggcttcatcacgacactctcggggcctcccgcacagcaagcgaaagcaaaaaggatccaaatggaactacttaaaataataagggtattttcgtcaacagaggaaaaaacttgtagcctataaataaaccctctaaccctcgtttcctcacttctcttcactctatcattctcctttcttctcttgcggctagggttttagcgcagcttcttctaggtacgttttcttatgccgtttcattagttttaattcttctattcatgcttatcctattggattgtgttgcctgctgaatatggttttctatatgttgtttatgtcttcaatatatctctgttttgttgatgaattttaattgtttcacatgaatttcatgtttttttcgaatttttgtctgagttattggttataatcggtaagatgtcttcaaaaaatgattttttttttcgtttggttttgtacctttgaaatactcagatttgatttgatttttgtggatttgtgtgttttttatattcaaatccatggatccagatctggttaaacttttgctttttgtttaagatttttctcaattctgatggatggatctataaccttttggttttaatcgattcaagtcgcattttttttttgagatggcatgataaatgtgtggtttaacccactggttttaattggttcaagttgcggattttacgggatgacatgataattgtttggtttaaccttttattttaatcgattcaagtcgctgattttacgagatgacatgataaatgtgtggtttaaccttttggttttaatcgattcaagttgctgactttacgagatagacatataaatgcgtgggtgtcttgaatacctttttcggatgtggtgtagtaacatatgtgtgggtgacttgaataccttgtttgcatgtgggtgtcttgaataccttgtttttgtggtgtagtaacttatgtgtgggtgtcttgaatacattgtttgcatgtggcgtagtaacttaattttgacgtgtcaatgcgtcaatatttttatttctgtatgtaatctgttttgtgttccctttgctctaatgaattatacctactaatagcttgttgtctgcgttgcttatagttgtatgcctgctttatatgaagtttgtgtgcaaggaatttggagattacccgatattttaatgttttaatggttatacatgtcttgtaatataaagcttgttgtgtgatttcttcttatatattgccgttacttttaattggtatgcataatgtaaactttgtttttagatattacccaatatattatttatgattatccatggtttgttgtatatgcgaacccatctcctttgtgtgatgtggctattttttggtttaagatgatgctaatgtatgcatttagttgttgctttttgcagatactcaaacgcagtt

SEQ ID NO: 37 - DNA, 2087 bp, Phaseolus vulgaris
  source 1..2087: mol_type = genomic DNA
catgcgactcctactccacctaaactaggctgatttttaccgcaatgattatttttgcatgcgccaccttcaccacctaatagactacccttcaccttcgcaggaaactgaaccgttttttctttgccttcctttggttcaaaccctccaccaagctccgagcagaaagagttaacagacccaactcctccaaattcgaccaagcttgccggaatcctcctcacgaaccactccctaatgatgatcatagtaacaaaacctcaatctcttcttcaaaagagacggtgcataaagtaccattaatttgcacttgcttcaccacctttgcatctcttcctaccgggattctaacgtgcagtcgagcgaactcaacaacctcccttgttatcgtggcgttgtcaacctcaactaacgaaccgacaagagacctaataaattcgaaacactggctactccacaacgccaacgggatacctctacagcgcactggccactccacaacgccaacgagatatctctacagcgaacccaaacaaatttttcacctaccacaaagctatcgtcccaagggacaagagactcgaatatcccatctaaccattctttatttttgacaactaacttctctaagaatcccacttcatcacaggataacaacacaaacttctcccctagagacctcaacttcacagaattaactccactcaaaataaaactttccttaatagactgcacattttttaactcaataagtctccctacaaagcaatcctctagccactttgtagaatccaccacgacttgaaagcagaccgtgctggcgtccaagaccttcttagaagcagactcgtgaccgtccctaaccacttgagcaaaggatctttgttgagccttctgcctccaagccttccttacatgttcatgtttccttacttcatccaactcgggtctgtgtggttccttcctgctaaacttaggtatatttacccgcagcttccaaagacctatccaattagcgtctagtttcctttataaaacgtgagcatccaacaccccttgaaatctcacgaacccaaagcgtcgtttaccagaatctaaccttcttgatatgaatacgtcgataacccttccccacctctaaaaaatcttccacatgtccctctccatgtactcatgaggaaagtttgtgaagaataacttcacttctgccattcttttggcaacgtctctctcttacaaccctaaaccccaaatattcagcatagaacagaatgctgagcatctgttagtttatgtctatgtaataggttgttttctttttataataagtttttaatatgtatttttttttctatctccttcttcttttcttcttttttgttgcacatttttttgctctgttttttatagcttatgattgcaaaaaagataagttcaataaaaccataatggatgacaaattatccatgcaaaattagatagaaaattagcaacaattttaacgcgtgtttgggtttaacgaacttgttttgaaaagttaatactccaaatttgttagtgtgttgacattaattgttttaataactattttgtgatgaaaaatgtggaaggaagacttctctacggtgttacttgccacccaaggctagatattgtttatgacttttttctaaaaaaagaatggtcttctattatgtacataacttgctacccaaggccagatattgtttataaacaaacccctcttaaacaaccacttaatatctctgctacacctacactctttctctttgaggctaaccactgttcagtgcagtttctttaaccaaggtttttatctcaattgtgtcttatgtatgaataattaacatattactgattcgtaaacttagtgtataaaattgactatgatggtgtgtaaccagttatcttttgtcttttgacttatgacttatgatttatgatggtgtgtaacttgatgtgacttatgacttatatgtgcctgctacaaactcggatcgtgagaaacaattgtttagattgtatgcaatagaaatgatgaatgttgattttaggttggagaagta

SEQ ID NO: 38 - DNA, 2306 bp, Trifolium pratense
  source 1..2306: mol_type = genomic DNA
ctcacggacttaacgggtctcattctattattattattgcctaccacaatttgtatgtgttatcctaagctactgaaccattttctctcttttattttcacaaccaatattcgtcccactagaccgcctaatttggttcggtgtcaattctggcatcaagtggttacaacttcccccccgattgcaattacgagatatcgaactgtggttatctctaccaatttcaacgtcaatcacttaaccaactaacaattggttacgaaaccaaattttaattgcataaaatttggtaacatatgcccggtcattgcagccattacaattctaaattgccaagttttaagcagatgataagctcctctcttttccaccaagaaacatcttaccactattgttaatcaaattaatatttattctgcttatatatatagaataatgctagcaatacactctttaacaaacacactccaacacactctcttttattggttgaaattcacatgggtcccataaaaaaatgtggacccatataaattttaaccaatagaaagagagtgtgttagagtgtgtttgttaaagagtgtgttgctagcactcctctatatatatatatatatatatatatcgaactcatgacctataaaatactatccaaatccctcaccactagatcaaacctagtgactttgcttaaatataattttaattcatgcaaaatatcaaaactttttaaataatctttgaacggctggctagatattttgctgacatgtcaaattcctgatgaattgaaagatgctaagtagactttgtcgtatcaatatagtcatttcaatacgccataactttttctttggtcaactaacctagtgactagaaatttcatcattaaaatggatgaatgagatgaccaaagttcgaatctcaaacctctgcatattgtaatacaatatcctgccaaccgagctaaactcaccgccctcaatacaccataacttaacatatggtcacaaatattaaaatgaagatgatgcaattatatgatcatgacttggggtacaagttcattgaatgactttatgtttatcaagtcaattcagttgcctgacctaatacaagtaaaccagagggcaattccgtcaaatgaaaaaaaaaaaagttttagtactatataaaccttagacctggaactcaatttatcatatctcactactactcttctctcaaaggttttagggcggcttatccttcaatgtaagtctcccctatttcattctaattcatatattcatctattcctatcatcgatcatcgattagtataagtatgtgattatataggattgttagaaattggtttgtttatgctttgacttatgcttttttttttatgatctgtttcaattttcgttttggtttgttatgataatcatgctgtgatttatgtgcctgtttatttgtgtttatgaatttagattgtgtgataatcgtttatgttttttgaatccgtagttttaatttgatgcatatatggcccgtttggatttagcttaattttgagcttttgagcttatgaaaataacttatgcaaatgaactttcatgttaattcataagtttagactaacaaaaaatgtattttataagttattttatggtaaactagcttaaaaatcttgtaataataaataaaagcttacttatttgcataagttgtttcacataaactcaaagataagccaatccaaacgggtatatgaatatgataaagatgttctgtgtctgtggttggaatatattgttgctgtttgatctggaacatgcggcttgacgtcctttaaacttgaatatttgacatagattgtgcttttttttatcagtaagaatgaccttgttgactgtattttatctattaattttattgttgaaatcagagttatatcttacttatgtattattggcagatcttttttcttttgttattttaagcttaaagacaagtgttttgtattttcatttccgatctaaaataacagacatgaacacgcgatcatataataaagttatcattctgagatatatacttagtgatttgttttttaatcagattttgtttgctcaaactgctgaaacacgtgtcttcctcccttgtgtcttcatcgcttgatattttattattgactgggcatttttatttgtctgctgcgtgatgttggcggttttaatattttatttctttcgaagcataattatgtattggtttatgtttggaatggcagatcttttgtgattttaagctcaaatccaa

SEQ ID NO: 39 - DNA, 2547 bp, Trifolium pratense
  source 1..2547: mol_type = genomic DNA
tataatataaaaaaatcatttttattcttgcaaacatatagattatcaatattaaaccaattgcattaaatcaaatgggataagaaatacaaaataattatctttgtatataacgtcaaaaatgaacctcctaatctaagtttaaattttatgcaacgattaaaatagactaaaactatatttacgagtatagataactaatctattgatactcatatgatattatatgaacattaacaatatataactattagtttagggcctaaaaattaatctattaatatagatctgatattttataggtataaataatatataagtaatattataggacctcaaaatnnnnnttttgaggcccgatgccgtcgcacaccttgcacatgtgtgcaagccgccactaatggtcctccaaacaagttcatatttgattccattttgtatcgtttttgcttttttacttgataaaataaaggaaaagctaatgtgtgtccttaggacacatgttagtgattcaaaaaatgaattgttttatcataattcatgtatttaattcactttttaaacttgatcacatattaattacacaaaatttaagagaatatttctatttttggattcttaatatcaagaccgatgaaataatactccttccgttccatattataagcacaaaaaatcactttttcatgtctcaaattataagcaaaaaaaatcaacttttatcattttcaaaataaatttttacttaatttactttctcttattttccccataatcaataaccaataaaaattctttttacatatttccataaaacttatttcaacaaaaacgcaaaaagtcattcttaaaattatcatattttactttttttaataagtgtgaatttttttttttatttatatcatgagacggagggagtataaccttggattattaattaattaattaattttctaaagaagaatccttcatccttttttattttgggattagccacagtcgatgaagagcccaaaccgatgttagtattcactttcagcaaacctcaataaacgacgtcgtcacatactctaaccttcatcttatgatagtattatcttatcttattattattatccttcttacttaagaaaactcactaaaagcatattccacacgcgaatcacccagcgcgtgacgacaaatctaagaaagacctagaagaaactcacgcgagtaattcacacgtatctttccttatcagtgcaaaacaagatcaacgttcaaataacaaagggtataacaggaaatccaaacaaatccaaggaccattttggaatatgaatattatctttctcactataaaaatgttcgaaccctagttttcttcattatcactcttcgttattccatagctcagtctctgtgcggctgagaattccgaacgagtacacatccttcgatttcatttctgctaaggtataatcctttgattcttatctttttctttcgatctgtgttgttgttctgttatatcatgatttttttttcggttcatttctgtttaatttccatgttttgattttgttttgtgtttgataaattttgattttgtgttaatttatcattttgttcggttcatttcttattaatttgcatgtgttgatagtggatgaatcaattttaaatttgaatttaactatgacttttttcatttcatttctgtttcatttttgagaaattttaattatggatgaatcaattagtgtatgtgatatttgatttgtttttagctttttgaaccgattgtttttcatgaatttatagatctgttgattttatgtttaaatcttataatatggtctagaaagttttgagcttttgatgttttggttgcttgattttggatctattaagataagtctttgactttaattcatgttgtttatttgatgaaaatgatgtttttattgtttagatttgattgtgtttgtttatgatagtttaaacagtgacattttttacatatgaaaagttattatctggttttactttttgtatggtgcaacatgaattcatgtttatctttcatggtgtttttcaaaattggattggatctagtttatatctattttgaaagttgtataaattacatgtttttgttatctatgtttgattagatcttatttggctaagcttgttttgaaaattttgatgtgagctgagtttgattgttatttggttaagctggttttgtatttgttgtttttgaacttttataagctgagttggattcttatttttgttaagcttgttttaattttatcttttgaactattgatgagctgagttgtatttacttaaggaagtgaattattgataagctgagtttgattattatttggttaagctggttttgaactattgataatcttagttgttttctggtttgttttctggttttaaactattgataatcaatagtggttttcaatttgatgaattatttttcaattggtgttagttctgaattgtgaaacttttgtgaattgcagttaagacatc

SEQ ID NO: 40 - DNA, 2498 bp, Trifolium pratense
  source 1..2498: mol_type = genomic DNA
atataaaattatagaaaaaataagaacaaaacaataaaaatattataaaaagttttgtcaaaaaaaaattatagaaaaaattatatgaatactagcgggtcaggcaggcgcgttatgtccaaaaaaatatatatatgtcttatgttaaggtgagtttgttaataaaagatttttgctgctaagaaaagatgtgtctgatattatggtaagtttgttaacaaaagattattgttgctactaaaaaaatcaaggatattgttgatacataaaaataggaatatggaaatagaannnnnnnnnnnnnnnnnnnnnnnnnnnnnnnnnnnnnnnnnnnnnnnnnnnnnctatcaaaaaaaaaaaaaaacataaacaatatttttgcaaaacaaaaaaatatctgttactagtatattaaaatcaattatcagcaaagttcctaacttatccttatggcttttaactacgttagaagttttatatttttcattttaatttcactaaaaaaaaatataaaaatatatataaaaaaagaatataagataaatacaaaaaacaaaaaaaaaatcctccgtatatagaaaaatataagataaatacaaaaaaaaaaatgatatgcataaaaatatgaacaaaacaaattatagatagaaacaaaacagaacaatctatacgcataaaaatagggggaaaaaaaacaataaaaacattatagaaagtttagtcaataaaaaataataatattatagaaagactatagaataaataagaagaatgcgtcaaaaaaaaatcgcatgaaaataagaatatagaaataattttaaaaaaacaatattatatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntactatatataaattatattttaaaagaaatataataattgaccacaatcttaaaagatttgattatggtcgaattaaaaaaaaaaagttaacttttttttttggtaaatggaaaaaaaaaatgtgatttgaatccttaattaataatcgttaatcttgagtaccacgttacgtagtatataaacctctctaagtctaacacttttcaaaaccctacttctctgctcttctttgcctccagagttttacagcaccttcctccttcacaaggtaacccttcgatttcgttttcatcgttttattcttattatcatcatcgattcatagtttattcacgttgtttcgattgcattacattataatattagatattgctttgttttatgtttctgatttttgttttcaatttgacctagtttgttagaacctgtgttcatgttaaatcccaaattctgaatacacattgtgtgataattgttttttgttcatgaatcagtacttttatttgttattaaagcatcaatcttttcactgagggttttgtataacatgaacatgtgattatactataataaagatgttattttttagttatatatttagggtttgttaatttgttttcaaatcgtaatgtgtatgcttaatctgttgaaacagtttgtgtatttacatcttgatattgaattattgacttgaattgtgtattctgttgttggtgtgttgttgatactatattttgtttttttttttagattgatacttatgtattgttttctttgttgaaacggccgatccgtttgtcatgcgctatttacagtagtatttttgtgatttctaactctttttcctttattctgtgggatcatacttatgtactgttttcattgtgaattggcagatctttttgttgttttaagcttttaagaacaa

SEQ ID NO: 41 - DNA, 2500 bp, Trifolium pratense
  source 1..2500: mol_type = genomic DNA
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttgagctggaggtattcatttatatatagttaagtcttgatttgattcatctgtttgattgaatcttatttggttaagctgattttcaacttgatgtttgattagatcttacttgttaaagctgattataaatttcatgttcgttgatgctgatttgtatgtttgattatatcttatttggttaagctgattttatactttatatttgattggttcttatttggttaagctggttttgaacttgatgttgttagctattactgagcagggttggattctagcttgttgatttgttgtgttgtgttcaatttttgttattttgcttattgaaattaattatttgtaaataatatgagttctgacttttgaaactatttgctagttgctgtttacataaattatgaattatttcaaatgaaataagttctgacttgaggaattttttgaattgcagttaagacatc

SEQ ID NO: 42 - DNA, 2500 bp, Trifolium pratense
  source 1..2500: mol_type = genomic DNA
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngttttgttttcaaaagaaaagtaattttgtgcttttcttcttcctgcgtgcatacttcttcttacttctttctttttcgtcagtagtttttatttctttgtggaaggaagagtgcaatagagatgcaagaaaatgtgttaatttcaaaaaattaggattatgtgttaatttcaaaatgtcagggtgtgcagttgcacaccctcaagtgtgtatagatccgcccctggttgaaggaactaacatgtgtttaaaggatgcaaccgcacattcaaacataggcttactatccattagatgtaaaataaagattttaatcgcatagaaaattatgaggtgcaagtttttcaaaactttctaaaacttaatccccactcttttccacctagatcacatgcacacgaggtttcaagacccataagaaatgcaaatagaggatcaaaagagtaacatatttatgcctattgttgatttttggattgttgtagtgacataaaatgactatatagagaatacaatagtaaaaacctcatgtaatgtgaggagtttttaatttttattttcgtccttttcttaattgaatcactaatttttttttattttattatttttaaaatttaaatattgaaaaactttattgttttgtcatatcaattgtagactttgtcttagacattagactgttttatgctattaactcggatgttatgagcatgtttgcagattcatccttattagtttttagcgaaattgaatttgtaataatttcgattgatgtattcctatgaatttgaatgaatgaatatctaaaaaaaactttattgtagaaattaaattaaaaaattatatatattttttaaaataatttagtgactaaattttttaagacgagtcaatggaaaattttgaatttgaattctaatttattgtaatgttcctaccaattgaagtatatttggaattccaacccttattttattacgggatattttttatttttgacaataattttatgggattttattatgtgattttgtttttggaaagattttattattctgtgatttaaattcttaaaatatgttttaccatttaattattttttggcacaaaaaaaaaaaaaagatttgaatccataattcagcactattctatataaaccctctcacccttccttatttcgaaaccctacctactctcattcattctcttcctaacgcagctttctccttcttcaacaaggtaacccttttttttttgtttcttatcactgattcatagctttattcgtcttttttacttcccaatatgaaatcattcatcttttcacccaaggttttgtactattttcatatatgatcataaagttgctctttttttaacataacggaaacagacatgaacatgcgattatatgataaacttgttctttttaataatatactttgttcctgtttcaattcatcattgtgaacttatataccgacataataagttttgtgagactgtttggaagaacttatgaaataagttttgtcagactgtttgggtgaacttatgaaactagtttatgacaatttttatgataagtgcttgtgctataaactgcagataagttgtttatccaaacaggccctttgtgtttgttttcgtatcatactgtgtttacttgatctgctgagtgtctttacctcgtgatattgaattattgacttgggttgtgtattttttcatttgtgttttgttgatagtgtagttttttttagggttttgtattttcatactagatgcttatatacatagacatgagcattcgattatatgataatttagtttacatctgggattacatgaaataaataattatatgttgttttgtttttgttttcaatttgttatttttgcacgtgcatgttaaatcacaaatctgaatcaagatagtttgataatcgtcttttgtctatgaatttgtagtttataaagacggttttgtatttccatactagactagatgcatacatatgagatgaatcgtttttttatagttgtaatttaagtgttttttgggatagtactaattatgtttgttttcttttgttgatttggtagatctttttgtggttttaagcttttaaggacaa

SEQ ID NO: 43 - DNA, 2154 bp, Vigna unguiculata
  source 1..2154: mol_type = genomic DNA
caaacagttcatccatctcccagaaaactttcattcattagagaaaaagacatacaaaagtacttacatatggtagaagaagaaaatatttaacaaaagaagaaaaacttgtacctttgttgtcttcttcaaacaattgatccatcgcccaaaaaatctttatttatcaaggaaacaaagtcgtacaaaagtatgtacatctagtagaagaagaaaagatttgacataagaaggaaagttcatacattttttgttgtcttcttcgaacacttcattgaaaacaacaaaagaagaataaacattgaagacaaagtaaagaaagtaaagatgcctttgtgtttatggttgacgaaggttgagacgaagcatgagaggaagataatgaaaaagattcagagtttaccaatgaatgatgaaaagtaggtttagcttgttagggttttggaaattgattattgattattgtctcacacaaacttttcattagaataacaaattaaactttcgagagtttcatctttcatcataaaggttcgttaaaatataagaaaataaaatctcaattgcaaaatatataattaagactaaatgtaaaaaatctttaattttaacatgtacaatttattttgtacatatttatttatctcactcattataacgcttaatatattttaaaatttagaacagaaaacaaaacataagtaaatttaaaatataaccggtgataatatttgtgaaattaaaatctacgaaaaaagaggataagatgtgctgctaagccatgatattaattatctgaaaagactattagtagcgagagattttattcaacgttgataatgaacaaatgcaaatgttgaaacgatgtcgttcatgtatcttaacctagtcacctagctgaaacgatgtcgtcatagtggcgcagctttcctgtttcgtccttgccaactggactatgtcgaacccacaaaccccgacatccgcgcaaacacaaagcgcgtgaaaatcaaccgcacaagactatcgagaaaccatatgcgtcaactccacgcggtcattccacgcgagtggcatacagtaacgttcaaataagcaaaccggggcataaacggaatccggataaaaccctaaggttattatcggaaacgaaaagtagctccaactatataaaactcttggaaccctagttttcattatcccttcctccgtgcttcctcgctctgtttctcagtctatctttgcggctgaggattccgaacaatcggccttcgtttctcgctaaggtagcagcctctgctctcttgtctcgctgattccgttcatctctgtttcttgtttacgatgatgattcttcgtatatatatattttttaatttggtttttaaattttatatttccggttctgtgtttgtggttcgttttttgtgtggattatctacgatttctaccgattaacggaagaggttctcgtttctgttttgaacctcgttgtgttcacagatctattgcttttgtgtcataattacgcgattttatctgttcttctttttagttaagatgttttcaatgatttattgtggatctcttattaccgaactaatatggctggttttcgctttcatgcaacagtgtcgatatttttaatgattttatttatgttgtctgaatctgaagcttgtataatattttaatttctatgcgaatgttgggatcctcggaagtttttgttgttgcccttttgtgaattttttgtaacatgattttttttttataaagttgtttttgtttctgctcaatagaaacttttcgggtatgagtgggtttgctgggagggtttttcgttatgttctttacgtttgtgcttcgcttaactgtttttttttttaatttattttcttatattagctggagatgtcgagttgaaacttgttcttaaggatttgtttatttttcttcttgattgaaaagatttttacgttgtgatgaatctgccttggtagatgcaagttgattcatatttcgcaactcttcttttaattaatacatgtgtgaagttgaactttaccttgtcatttgttttgcgtgataattcactttctagcgattgtgagttctgacttgtgtttctttgaattagtttcagtttaactcatc

SEQ ID NO: 44 - DNA, 2235 bp, Vigna unguiculata
  source 1..2235: mol_type = genomic DNA
cgtggttagtcactgaaatgaccaatttagatatcaatttatagaataacaaattattaattactaaaatagtcagtattatgaataagaaattactaaacattaactactaagatcttgactatccaaaaatttcataaattggtatataaattaatcaccattatttttaataatcaataattttttattttataatttgatatataattgatcaccataatattaatgatgactaacaaactatggtaattaagggaaagattttcttctagtgaaatatggtcattaattaattattacagataatttttttggtagtcaaaacattaacaatttatgtttaatgatttttttatttatagtgactattttagtaactaataattttttatttataataattattttaataactaataaacttttgatttataaattgatatataaattaatcattataaattgatatataaattgattattatattaataactaacaaactaaactatggtaattaaaattcatcattatatttatagaatttggatttcaaattattatgaaagaagatattttgttttataaatctaaataatttattaagttcgttacttaatatgttacttaatatataccgtttaaacaaaaattaaccaatcacacatatttcccgtttttttagttttagtttgttttatttaaacggtaagtcaacatcatcgattgttaagatttattatataaaatattttcttttaatggtaggagggatgattcaaattaactttatttttgtaaaaggaattaataaaagaaaaagaaattaaaatcatgttcatacataatacataaataaacataaaaaatatatgatgtaaaactaccctatatgtatgacttatatcctaattaaaagtaaaaagtaataaaacaaaatcttaactaatagttgccctagaaaggaaaaaatcactgaactgtactgtgcggcgtcgttttggtagccgcataattgaaaggttacctagggcttcgtcacgacattctcgggcccttccgcaccgccagcgaaagcaaaacggatacaactggaactaggtaaaatactgagggtattttcgtcaacacgggaaaaaacttgtagcctataaataaaccctctaaccctcaatccctcacttctcttcactctattgtttgcctttcttctcttgcggctagggttttagcgcagcttcttctaggtatggtctcttataccgatttaagaggtttattccttagattcatgagtatcatattggattgtgttgcctgcagcttatggtttcttgtatctattgctgatgtctttaatttatccaagttttgttgatgaagttttattctttcacatgaattttatgttctttttgatttttgtttgagttgatgattttaatcggttagatgttttcaaaaaaaggattttttttttcggatgcttttctagctttggaacaactggatttggtttgatttttgtggatttgtgtgcttggcatattcaaatccctggatccagatctggtaaacgttttgttgtttggtttgagatttttcttatttctgatgaatagatctgtgaactttttgttttaactgattaaagtcgacgattttatcaggtcttggtgagtatttatgggatgccatgttaaatgtgtggttgtcatgaattatgattgcatgtggtgttgtatcttattttaaacgtgtctataatttgatctgcgtgagtatttagcttgtgttgcgttcgatttgctttaattgattatatccgctagtagtttgcttttggatttgtttttaggtactatctgatatttttgtatgttatgcatgtttttgtttttaggtgtcatccaatttttttatggttatgcatgttttactatgtaaaattttgtgatcgggttttcgatattgtccgataatttgatggttatccatagttttattcttttacgggcattaccccttattttttttttgtggttatgcatgttttcatatgtggaacttatgtggttggtttttagatattacccgatattttctattgctatgcacggtttgttatgcaaatggcgaagctcaccttatttatgttagatgtggctatttattgatttgcattctaactcttgttttgacggttgtttttttgcagatactcaaaggcagtt

SEQ ID NO: 45 - DNA, 2200 bp, Vigna unguiculata
  source 1..2200: mol_type = genomic DNA
tcttagacttcaataatttattgatgcttctctgttgatatatatatatatatatatatatatatatatatatatatatatatatatatatatatatatatatatatatatatatatatatatattattgaactacgtttagttaactacttttaatgtcatttttcaatcatattcttcttagtcacataacttaaatttaatatatatatatatatatatatatatatatatatatatttaaatagatcgaaccaattcatttggaccgacttatattaaaatattataaaaaataattcagaaaaataactaaaatattttaattttttaagtgtttgacatttgaaacatatatttataaaacaaaataacaaaactttggtatttaaataaataaagtggttgaatgctttgatatatatatatatatatatatatatatatatatatatatatatatatatatatatataaaagatgttggttataatcttttacatatattcaataactaattatattttatacttattttaattatattttgagtgtttattcagggtttaaatatacacatccatatttaattttcttttcatcattccacgtaccattatattactttctaaatttgtacttatctgaacattgaaaatcatctgcaagtgaatatattttcagttttgattggtgctctctcattactagattgatagttcgtcctttgaggatctcatacttccatctcagaaactattcatcaatctaaacacacattctttattagtaatttagctaagtttaatataaaaagaaattgataagaaaaatccattgagatgatgtcactgtataatagaaaaagcttcgttagtcccattagaaataatatcacaaattccatgttgtgaaactcaattccagattgagtgtctccacattgtgcaaattttaatttttgtaaaaatcttgcagacagattcgaattttgcgtgagagagaattgagaagccatgacacattcatcttgttaatgtggtgtcattccacatatgaatatgtttgataaggttcagcaatggaaccattacaaaaacttgtttttgctaatatgagccatcttaatggcctttatccaaaaactaaaattctcagcatatcaaacacattactaactataaatgtctaatataaaataaaggttcctagtaggcatttaaggttaatgacagttttaacgggtctttgtgtttaacgaacatgttttgaaaagttaatactcactttgaataatttgttagtgggttgacatcgattttctttaatattatttttgtgatgagaaattttgaaggaagacttgtttctggtgttacttgcaggacaaggttagctattgtttctgactttttctgagaaaataatgattgggaaattattttttaacctttataattttaacttacaatttaaaactatttaaaaaataaaatttcaaattataaaaataattgttaagatttaatagtcaaataatattaacggctgagaaaataatgattgggaaattgttttttaacctttataattttaacttacaatttaaaactatttaaaaagtaaaatttcaaattataaaaataattattaagatttagttgtcaaataatattaacggcgttattatatataacttgctgcgcaaggctagatattttctataaataaacccctctaacccctctcaaaaaagcacttttctctgctacacgcacactctttctcttcttgaggctctccaggggtttatcgctttcaccaaggttatatctcaagtgtgtcttcttattcatggattattaatatattaccgattcgtagacttgttaactcagtttagtgtataaaatttgactatggtggtataatcagttctgtcttttaacgtttttgatctgggatttatgattttatgtgcctgctacaaaccctgaatgtgagaaaatcgtgtaaatttgattttcttttggttgttgttgatgttgttgtatatacatgtaggtgttgatttctttttacggggtagttgtaagtagtctttttatgcaataatttgtgtatttgtaatggttgtataaatcagtgttttagtgtatgcaatgcaaaatgataactttctttttaggttggagaagta

SEQ ID NO: 46 - DNA, 2100 bp, Vigna unguiculata
  source 1..2100: mol_type = genomic DNA
catattcaatattttgattttatttcaaaaataataaaatacgatgtgatatatatataaatatatatatatatatatatatatatattaaaaaatgtataaaaatttaaaaaatataataaatgtgattgttattatgtaaacccaatttaaaattatatttattaaatttatcaatataaataattataaattattgtaataaaaatactcatcttttatacattatatatttcgttgataaaaatcgataaaatattctaaaatactggtacaaacacatgttaaatacaaaatcgtgattcaaattggaatatcgattcatcgttgttttataataatgtttttcacacataaaatacaaatctattaaaaatttggattcaaatgtaaaacaatttaaagacccaaaatgattatatccaacttcataacttttcattcttcatccccaaatatacattattatatttttgggttaattatatcattaattatattttttgttttagtccctcattccatgtccctttcgttttacaactcatgaatttaatctgaatattctaatgagtttatttggtattagtataattgtaggactaatatttaaataggtattattattttgacatacaataaaattttgtacataggttacatttattattttactctttaattttttttcttcttttgcaaattaaaattattttttttaacgaataaaatattttaagcacttaatataaaaaaatcactcatttaaatatttaattagtaggatggaacctactttcattttgatatcttaaataacttacaaaattaagtattgatatttatattaaaatataatgataaaattcattaattaaattaacgactttattaatgttgtgtgaaatagatttaattaagatacttttttaaaaaataaaactaaattaacacattttttttagaaatagatatcaaactaaaatatcttaacaaaaataatacgatcttactaattaaatccttatttaaattagaaaaggttttctatcattagtaacaaccctaaatcttttttgtatcttgtgccgtcaaagaaaataaaccaactaggaattggcgtaaaaagtgacatgatcttctacgattttttgcaacacgtactgtacacataggaatatctaaaccattaattacactctctgtttctagattaacttattgacggctgagattcggctatatggtgtagacaaaagaattggccgcagaggatctggtttggcgagaggataggttttccatcgacgccacgtcaaaagaacagggatacaactggaataaagtgcagagagttaagggtagtaccgtcaacaaaaattagcaagttgcagtctataaattctcggtccctcttagtccaacaattttctttagtacactctcttctcgcggctagggtttaagagcagcttctttctcttcaaggttatttcttgctattcactttcctttcattgcttccttcatgtgttagcttttgtcttagttaattcctcgacgtttacggaaacgattgttattatccgatttgattttggttctgctttatctctctgctgaatggaaattaccaatccaaacagtgcgattctcgtgtcaattcgaattatttgttttcttggttcttcatctatatatatgttgctgtcgatcaatttttttacctgtgtttggtgtcttgcatggtggtgtgggtgcacatataatcctccatcttcatatcgtattgttattttagtttgagccattacatgcttcgtattacaatgaagtctatatctagtttttactcaatctttaataattgggggtctttttttctgagacatatcctaccgatttgaaaattattttttatttttgtcagtgccgtgaagtttttggtgtatgggtcatctcatttcattttatctttgaggattagggttaacgtttgggggggaagtatgtgtaatattttactgatttattctgttcttggatgattgattgttgtgttgattggccagatcttaattgtttcaatttagaggacta

SEQ ID NO: 47 - DNA, 1061 bp, Arabidopsis thaliana
  source 1..1061: mol_type = genomic DNA
ttgcaaacagaggagatatactaagcctttggaagagtgtatagatgagctagaactctacacacttggtcaatactagatttatctgcatcaaatgctttccaccagggtggattctctgggagaggtacttgaaacctacgtgcagcagcatacacgaccccacaagccagaacctcacttctgaaccttacacaaagggttgtacgcagactatcattagccaaattccaagcttcttgcctcaattccggaggtgtttctagtgttcaacatgacaaacaaaacccatctctttcagtatatgtctctcagttgtgcttaattcaaatttcaactcagagaacttcttggcatacttatccagattatctaatgatctcatctaatggtaattcaactttcagtataagtctcgcagcaaactatctttacatcaaatttttaacaactcaatgcacaaaatacttttcctcaacctaaaaataaggcaattagccaaaaacaactttgcgtgtgaacaacgcgtacacgtccctacacatacctgtcaatttataattggctattgcttccacgccttagctttctcgtgaccgaccgagtcgtcctcgtcttttttgcttctataaatcaaatacccaaagagctcttcttcttcacaattcagattccaattttctcaaactctaaaatcaatctctcaaatctctcaaccgtgatcaaggtagatttctgagttcttattgtatttcttcgatttgtttcgttcgatcgcaatttaggctctgttctttgattttgatctcgttaatctctgatcggaggcaaattacatagtttcatcgttagatctcttcttatttctcgattagggttcgtatttttcgcagatctgtttattttcttgttgtttccttgtatttgatccgatttgttgaaagaatttgtgtgttctcgattatttacgctttgatctgtgatttttatctagatttggtgttagtttcttgtttgtgcgatcgaatttgtcgattaatctcggtttatctgattaacag

SEQ ID NO: 48 - DNA, 2548 bp, Pisum sativum
  source 1..2548: mol_type = genomic DNA
gttgctgaatcagaagttgagtgaggtttggaggcaactgttgggacaacggcttagacaaacattttgatgctacaatttatccaatttttagagggttttcatttacgccctatatatttttcagaaattttatggaagttcaaaagttttcttgttgatgcgtctattacgttctttcatttgatctaatatgagtgtgttcgaaaattatctttaggagattattatgaaataaaatagaaagagtttgtttgaaagtaaactttaggacactgaaaatagaatctctgaaaaatatataggataaaatgtaacgagtaattctacatttttataaattttggaaccactcaattttgtcaacgagtttcagtcttctgctagagtttcggaaattattaccgctttcttcttgtcactgttctgtatttatataacatcatctgtgagagtttcgaaaattgttacagctttcttctgaccactgttctgtatttctataacagattaaatgaatctaaaaatcaaaatctacttataaaagagaatagagggtatatacacgtttattatttaatgaacccaaatgaattgtcttctatatatattagtttatttttagtcatatccaactttatgttgcaatcttaatagtggactaaaaaagatctaaaaatattaattcacaagctaatcaaagcatgactactatacctaatgttgttttaagaaaaaaatttcacaacataggtaaacatataactaaatttttatattgagttttactgtatacttataaaatatttgacaattttttttcaaagatacattgattagctgttctcaaatatccaatttcaaatacataagacaatacttttcaaacacaaattttatttgatgtcgggcttatcattttgagaaattgtctttttatagaagttgagcacacttaattttattgttcatttctttattataacttcacgatgtgtatgcaacattagggcagtttgttttaactttaagaaaataattttcttttaaatggattttacaaaaatgttttttaaaatattataagtttttctaaatttatttttgataaattaaaaatttgaattattcattctataatataaatatacattattaaagatcaaaacatagtcaaaatcacaattttttcaaaaaaattatatcttaaaaataatttttatgaaaagctatttgaaatagcttcaaaattaagtgattttttaaaattttgatatctaaattttttttcgataaaatgatgaaatacctaaaataacattttaagaataattattcaaaccaaatttttatttgaagtttttatgaaaagtttttttataaactttatttagattaaaatatgtaacactgtaacaatcatttaaaaaaaaaccaaaacaaatggatcctaaattaattaaatttttttttgagatttttctgatacattccaatttatattatcggtgctcatcccaaattcactatatcatttgtattcttttaaaaattaattttggcattttttagttatacgcactttccacgaaataagccaacagattcaagacgtgtttggatggcaaagctatcatcttatccgttgccgaagacgactgtgataccaaacattgccgtgtcaaaatcgtttatagctcagatttgtccacgtgtcgcgatgtgattggaagagctgccgttacttttacgcaacctcgaatcattttaagctataaattcgaaagcccaatttcacagctttcatatattcatatactttcgtttcgttgcagagaagagaaaccctagcacactgaatcaaggtatcttctacccttcatctttttctcacgaagatttcgataattaggtttctgtgttctctcgattgaataattattggatcaattttgatctttgtttggtttatcagacttcattattagggtttagtcttatttttaatcgattgttgttgattagggttttatcttttttttaatcgattgttgaatatgttagatatcattttgatctaattgcttatttcattttactctattcacaattgtatccttgggaaattttctttgattaattggcaaatttcgttcaatatttttatgatcgaaggacgtccatttctgtttttagggtttgtatactaaatttctcatagcagaggattatatagcttagttatagaaccttctttatttagattagtccaggggtacttgttgcctttatatatgcatcatgtctctagttttgtctgaatttttttcttctgtatatgtgtaatttagtattgctacccaaattaattattgattgattaggggaacttgaagtttataagatgtttcctgtaaatagaagtggttatattggttgattttatgcttcttttgtttgttacatgtggtttttctgtgttgtaatgatctttatagataatgtgaatacaaatttgtgattgatttgtttgtttctgataatatag

SEQ ID NO: 49 - DNA, 2604 bp, synthetic construct
  source 1..2604: mol_type = other DNA
gttgctgaatcagaagttgagtgaggtttggaggcaactgttgggacaacggcttagacaaacattttgatgctacaatttatccaatttttagagggttttcatttacgccctatatatttttcagaaattttatggaagttcaaaagttttcttgttgatgcgtctattacgttctttcatttgatctaatatgagtgtgttcgaaaattatctttaggagattattatgaaataaaatagaaagagtttgtttgaaagtaaactttaggacactgaaaatagaatctctgaaaaatatataggataaaatgtaacgagtaattctacatttttataaattttggaaccactcaattttgtcaacgagtttcagtcttctgctagagtttcggaaattattaccgctttcttcttgtcactgttctgtatttatataacatcatctgtgagagtttcgaaaattgttacagctttcttctgaccactgttctgtatttctataacagattaaatgaatctaaaaatcaaaatctacttataaaagagaatagagggtatatacacgtttattatttaatgaacccaaatgaattgtcttctatatatattagtttatttttagtcatatccaactttatgttgcaatcttaatagtggactaaaaaagatctaaaaatattaattcacaagctaatcaaagcatgactactatacctaatgttgttttaagaaaaaaatttcacaacataggtaaacatataactaaatttttatattgagttttactgtatacttataaaatatttgacaattttttttcaaagatacattgattagctgttctcaaatatccaatttcaaatacataagacaatacttttcaaacacaaattttatttgatgtcgggcttatcattttgagaaattgtctttttatagaagttgagcacacttaattttattgttcatttctttattataacttcacgatgtgtatgcaacattagggcagtttgttttaactttaagaaaataattttcttttaaatggattttacaaaaatgttttttaaaatattataagtttttctaaatttatttttgataaattaaaaatttgaattattcattctataatataaatatacattattaaagatcaaaacatagtcaaaatcacaattttttcaaaaaaattatatcttaaaaataatttttatgaaaagctatttgaaatagcttcaaaattaagtgattttttaaaattttgatatctaaattttttttcgataaaatgatgaaatacctaaaataacattttaagaataattattcaaaccaaatttttatttgaagtttttatgaaaagtttttttataaactttatttagattaaaatatgtaacactgtaacaatcatttaaaaaaaaaccaaaacaaatcgatcctaaattaattaaatttttttttgagatttttctgatacattccaatttatattatcggtgctcatcccaaattcactatatcatttgtattcttttaaaaattaattttggcattttttagttatacgcactttccacgaaataagccaacagattcaagacgtgtttggatggcaaagctatcatcttatccgttgccgaagacgactgtgataccaaacattgccgtgtcaaaatcgtttatagctcagatttgtccacgtgtcgcgatgtgattggaagagctgccgttacttttacgcaacctcgaatcattttaagctataaattcgaaagcccaatttcacagctttcatatattcatatactttcgtttcgttgcagagaagagaaaccctagcacactgaatcaaggtatcttctacccttcatctttttctcacgaagatttcgataattaggtttctgtgttctctcgattgaataattattggatcaattttgatctttgtttggtttatcagacttcattattagggtttagtcttatttttaatcgattgttgttgattagggttttatcttttttttaatcgattgttgaatatgttagatatcattttgatctaattgcttatttcattttactctattcacacgtaattcttcagatccgtttgttatgattaaattttcagatctgattatgtatcaattgtatccttgggaaattttctttgattaattggcaaatttcgttcaatatttttatgatcgaaggacgtccatttctgtttttagggtttgtatactaaatttctcatagcagaggattatatagcttagttatagaaccttctttatttagattagtccaggggtacttgttgcctttatatatgcatcatgtctctagttttgtctgaatttttttcttctgtatatgtgtaatttagtattgctacccaaattaattattgattgattaggggaacttgaagtttataagatgtttcctgtaaatagaagtggttatattggttgattttatgcttcttttgtttgttacatgtggtttttctgtgttgtaatgatctttatagataatgtgaatacaaatttgtgattgatttgtttgtttctgataatatag

SEQ ID NO: 50 - DNA, 2000 bp, Solanum lycopersicum
  source 1..2000: mol_type = genomic DNA
atttgaaaaggaaggacaaaaatcatacaattttggtccaactactcctctctttttttttttggctttataaaaaaggaaagtgattagtaataaataattaaataatgaaaaaaggaggaaataaaattttcgaattaaaatgtaaaagagaaaaaggagagggagtaatcattgtttaactttatctaaagtaccccaattcgattttacatgtatatcaaattatacaaatattttattaaaatatagatattgaataattttattattcttgaacatgtaaataaaaattatctattatttcaatttttatataaactattatttgaaatctcaattatgattttttaatatcactttctatccatgataatttcagcttaaaaagttttgtcaataattacattaattttgttgatgaggatgacaagatttcggtcatcaattacatatacacaaattgaaatagtaagcaacttgattttttttctcataatgataatgacaaagacacgaaaagacaattcaatattcacattgatttatttttatatgataataattacaataataatattcttataaagaaagagatcaattttgactgatccaaaaatttatttatttttactataccaacgtcactaattatatctaataatgtaaaacaattcaatcttacttaaatattaatttgaaataaactatttttataacgaaattactaaatttatccaataacaaaaaggtcttaagaagacataaattctttttttgtaatgctcaaataaatttgagtaaaaaagaatgaaattgagtgatttttttttaatcataagaaaataaataattaatttcaatataataaaacagtaatataatttcataaatggaattcaatacttacctcttagatataaaaaataaatataaaaataaagtgtttctaataaacccgcaatttaaataaaatatttaatattttcaatcaaatttaaataattatattaaaatatcgtagaaaaagagcaatatataatacaagaaagaagatttaagtacaattatcaactattattatactctaattttgttatatttaatttcttacggttaaggtcatgttcacgataaactcaaaatacgctgtatgaggacatattttaaattttaaccaataataaaactaagttatttttagtatatttttttgtttaacgtgacttaatttttcttttctagaggagcgtgtaagtgtcaacctcattctcctaattttcccaaccacataaaaaaaaaataaaggtagcttttgcgtgttgatttggtacactacacgtcattattacacgtgttttcgtatgattggttaatccatgaggcggtttcctctagagtcggccataccatctataaaataaagctttctgcagctcattttttcatcttctatctgatttctattataatttctctgaattgccttcaaatttctctttcaaggttagaatttttctctattttttggtttttgtttgtttagattctgagtttagttaatcaggtgctgttaaagccctaaattttgagtttttttcggttgttttgatggaaaatacctaacaattgagttttttcatgttgttttgtcggagaatgcctacaattggagttcctttcgttgttttgatgagaaagcccctaatttgagtgtttttccgtcgatttgattttaaaggtttatattcgagtttttttcgtcggtttaatgagaaggcctaaaataggagtttttctggttgatttgactaaaaaagccatggaattttgtgtttttgatgtcgctttggttctcaaggcctaagatctgagtttctccggttgttttgatgaaaaagccctaaaattggagtttttatcttgtgttttaggttgttttaatccttataatttgagttttttcgttgttctgattgttgtttttatgaattttgcag

SEQ ID NO: 51 - DNA, 1420 bp, synthetic construct
  source 1..1420: mol_type = other DNA
cctgaacttgtattattcagtagatcgaataaattataaaaagataaaatcataaaataatattttatcctatcaatcatattaaagcaatgaatatgtaaaattaatcttatctttattttaaaaaatcatataggtttagtatttttttaaaaataaagataggattagttttactattcactgcttattacttttaaaaaaatcataaaggtttagtatttttttaaaataaatataggaatagttttactattcactgctttaatagaaaaatagtttaaaatttaagatagttttaatcccagcatttgccacgtttgaacgtgagccgaaacgatgtcgttacattatcttaacctagctgaaacgatgtcgtcataatatcgccaaatgccaactggactacgtcgaacccacaaatcccacaaagcgcgtgaaatcaaatcgctcaaaccacaaaaaagaacaacgcgtttgttacacgctcaatcccacgcgagtagagcacagtaaccttcaaataagcgaatggggcataatcagaaatccgaaataaacctaggggcattatcggaaatgaaaagtagctcactcaatataaaaatctaggaaccctagttttcgttatcactctgtgctccctcgctctatttctcagtctctgtgtttgcggctgaggattccgaacgagtgaccttcttcgtttctcgcaaaggtaacagcctctgctcttgtctcttcgattcgatctatgcctgtctcttatttacgatgatgtttcttcggttatgtttttttatttatgctttatgctgttgatgttcggttgtttgtttcgctttgtttttgtggttcagttttttaggattcttttggtttttgaatcgattaatcggaagagattttcgagttatttggtgtgttggaggtgaatcttttttttgaggtcatagatctgttgtatttgtgttataaacatgcgactttgtatgattttttacgaggttatgatgttctggttgttttattatgaatctgttgagacagaaccatgatttttgttgatgttcgtttacactattaaaggtttgttttaacaggattaaaagttttttaagcatgttgaaggagtcttgtagatatgtaaccgtcgatagtttttttgtgggtttgttcacatgttatcaagcttaatcttttactatgtatgcgaccatatctggatccagcaaaggcgattttttaattccttgtgaaacttttgtaatatgaagttgaaattttgttattggtaaactataaatgtgtgaagttggagtatacctttaccttcttatttggctttgtgatagtttaatttatatgtattttgagttctgacttgtatttctttgaattgattctagtttaagtaatc

SEQ ID NO: 52 - DNA, 770 bp, synthetic construct
  source 1..770: mol_type = other DNA
cgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctct

SEQ ID NO: 53 - DNA, 387 bp, Arabidopsis thaliana
  source 1..387: mol_type = genomic DNA
ctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgatt

SEQ ID NO: 54 - DNA, 2000 bp, Solanum lycopersicum
  source 1..2000: mol_type = genomic DNA
acaatcaaagctcattttcctatcaaggtaaaccggtaaagcagtgggtaaaaaagggttggctcgtcgaaagcccgacctctttggtccaaaaggattcaatttctcacctatgttagttcgacgtgcttgttgctctgattaagattatcagaacatcctgaaagattctcttgaattgcttgacttgcaagtggagaataacgagtctcacccactttttgagctgaaaaattagctagtatgaaatttcttaaacatgcactcgtagtgcttaagatacaacagtcttgacctgatccaatgaaaaggaaagctaaaagagccccctcgataggtgacttttctttttgaaacatgtcgtataaaaaatcgtttagttaggaatatacataaaattttgacttattataaggatttatataactaataaaaataatataataggaataattttaaacaacagaaaaaaataaaaaagagatacatccaatttttcttcattcaaattatgtaaattttatttttagaaattacagtcaaataaaaatttaaaaaataattttttaaaattatacatgccatgaatttgaacaaggaaaaaaaaagaggagaagcatctaacaaggctctaataatttgcgtaaccgtgtcttctaaaatatgcgaagaaattgcgtaagcgctgagccataggcccatacgtttcctctctgtggcggcaaagcggttactataaatacagatcttcctcttctcgacctactccccaaatcattcttcttctctagcgcaacttctctcggaaaaaggcatctcctcctcctctcgttctctcgataatctccttacgcactagttcttcttctcaaggtaatggtctttttcttctatcgattcaatcgtttgttgaagagatttagatttatgcaggtttttgtatcataaatgtatgaacagtattatatgaacggaatttacctttgttttgttgtttatcgattagatctgcacggaattagtcgatttgagaagtttttaggatggattatgtatgtttttttctgttgatgatgttatggcgtttaatttcgtttgatttgctagagttttggtttccatttggtagaatttgttgaagttttgtggtatgattagtttgtgtcgtatctagggtttttttatcacaatttgaaatggtgatgcttgcctttctgttgatgatttatagccttgaatttcgtccatttgcttgattttggtcactgtttaatagaaattgttcaagtttccaggtttgattaatgggtcctgtctagggatatctatgaataatttgaaaaaaacttccttttgatgtataatatatgctcttttttcttttatgttgatgatgttatagtcttgaatattgtcgtgttgttccatttggtttctatttgattgaaattgttcaagtttcccggttttattgtttgtgtcctagggatgtttatgattgaattacagaactaaattgaatggatgatgcatacttttgctgatgatgatttatagccttgaattttgtagatttgcttcatttattgtctccatttaatgaaattgttgaagtttctagcctctaagtttgattgattgtgtcttgtctagtttttttttttgcgaacaaattgaactagatttcaaacaaattgggagtctccaattttttgtttgttaactcttattgatctgttttttaatgtttatttcttgtacgggcactttgttattctctcctgataattctacattttgaacttgatttgatctacaatatatccaatgggtttgcatccctctaaaatgattagtatagtttgcactaggtaggagggaaattgcagaacctttgttttactttattttctttttggatttgcaaagattgctagttttgccatctctgcctgatcttgaaatagaagttttgacagttgtgcttcaatatatttcag

SEQ ID NO: 55 - DNA, 1851 bp, Solanum lycopersicum
  source 1..1851: mol_type = genomic DNA
agttaacgaaaaatttaattatttaaagtaagtatatttttcaataacaatgccaaaacctctaatattcttaaaattaacaattggcaagtgatttttaattagtttgctattactattgttgttattcttactactagcattattactattaattaattattattatcatcactatcattattagtaaaaggtttttttatgtttgtatcttttaatttaatatttaaattaaatacatttacatttattttgcaatatttaaaaatgattagtttgtcaaatagtagagttcatttaaaattcttcagccatatagttctatttttaagctagtcgacttttttttcttactgaaaattaatatttttttctttttgaaatactaatacatctaaatttaacaattgccaaagtgatttttaattagcttgctggctaatcacaataaaaattactctcctttactatataagtaaatttttattgctatatttgttattattattattattattaatatttattttctacaaatttaataatattttattttatatcattttaaaaagataagtaatgaaatattaagaattcgtttataattcttttgcaggtgggtttctatttgtaagctaatctttttcagttatcctttttttaaaatctttattattattatagctatatcttttatcttttaaaattaacattatctattaaagataatttcaataaaagagtaaaaattaatttagagttctactgtcttcaaatttctattttaaaaaatacttttaaaacttgatgtattttttacgtggtttttcactatgacttaatttctgttttattataatatgtataaatataaaaatagattttccataacatattataaaaaatgtaaggggcatttacgtaaatagatagacttaaaagaggcaccgagtgaaccctaattctcatcgttgagactataaaatgcccattatcccattcgcacagtctcttcattacttttgctgttatttctcctcagctgtgccgcatatcgcctaatttttcttctctaaggtttcatcatcttcaccaatttctttaatctcgattcaattttttatgtttgatctgttattgttctgtcactacatgtgtttttcagttgttttactagatgattttcactgtcttcttgttagatcatacatatattgaaaatgttttggattgacttttttgtattgtgaatatctgttattgtttgattgttgttcagtatttacacacccgatctgtgttatgagcttggtcataactatttctctgtatgtaaatacagatctgttaatgtttgtaatcaatttttcatatgcactgttgatattgttctctctcctgtcctgttatatgttgatatgattcggtttttgtataacttgaactaaacactagtcctaaatgttttttttactatttaagatttatataatatggatagattttttgagttcctagtctctgaagaggttaagcttgctgtagttgtttaccagttgaggtgcaatactaaaaatcaattcaattactgatattttttgctgtttaggtttttgacaaagtactttaatttgctttattgaactaaaaacgtagtcctgaattcattgcaagtgtgaaagctatagttcattgtttttgttgcaattcttgaaaaattaattggtcaagctataatggattttactttttctgttttaatattgaatttgctgaatttatgaatgggttgcatggtttttgaaatatgttgttgtgtgttgtgtaaatgcagtttcttagtgtctcaag

SEQ ID NO: 56 - DNA, 1306 bp, Arabidopsis thaliana
  source 1..1306: mol_type = genomic DNA
atcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacag

SEQ ID NO: 57 - DNA, 912 bp, Glycine max
  source 1..912: mol_type = genomic DNA
caatataacaacgacgtcgtaacagataaagcgaagcttgaaggtgcatgtgactccgtcaagattacgaaaccgccaactaccacgcaaattgcaattctcaatttcctagaaggactctccgaaaatgcatccaataccaaatattacccgtgtcataggcaccaagtgacaccatacatgaacacgcgtcacaatatgactggagaagggttccacaccttatgctataaaacgccccacacccctcctccttccttcgcagttcaattccaatatattccattctctctgtgtatttccctacctctcccttcaaggttagtcgatttcttctgtttttcttcttcgttctttccatgaattgtgtatgttctttgatcaatacgatgttgatttgattgtgttttgtttggtttcatcgatcttcaattttcataatcagattcagcttttattatctttacaacaacgtccttaatttgatgattctttaatcgtagatttgctctaattagagctttttcatgtcagatccctttacaacaagccttaattgttgattcattaatcgtagattagggcttttttcattgattacttcagatccgttaaacgtaaccatagatcagggctttttcatgaattacttcagatccgttaaacaacagccttattttttatacttctgtggtttttcaagaaattgttcagatccgttgacaaaaagccttattcgttgattctatatcgtttttcgagagatattgctcagatctgttagcaactgccttgtttgttgattctattgccgtggattagggttttttttcacgagattgcttcagatccgtacttaagattacgtaatggattttgattctgatttatctgtgattgttgactcgacag

SEQ ID NO: 58 - DNA, 2262 bp, Glycine max
  source 1..2262: mol_type = genomic DNA
cttggaattatgggattggataaacgtttactaaacatggaaagcgtttattaaaaaaattaagcatttattattccatttttttatagaaattattccatattttattcatggtgtaattaatattcttttcccataacaacttttaattactcaaacttatctttaggggacattttggtagaagaaaagttgtttccatcctaattatattgcaaggtggctgcattcatgattttaagaatatatttttgtagttcactgtaaaattattgaaaatatttacaattttttttcggaattaaagattttcattttcaataaaatactaagtatatgagtatacttttgtactacattatttttcttttactattaattacttttgatcttttccattaatttacaggtttattgaattttcgtatgccaaccttttaaattttattttatagtcatttttctaaattttgcaaacaaactatagcaataaaaaaatagactagtttttaataaattacaaataaacttatttttgctacacatttttttataacatatttctttacacgtgtgctgttgttggaataaagaaaaaaaaatgcattaaccaaatactatgaagtagtttgattttagatcacttgactaagactaaaatacatgcgcagctaacaccaaaagtctacggggtttaggttgaatccaaactgaaagtttgagaaaatcggtatattaattgtcatacttcctccagacttaaataatataagttaaaaaaaatagacctaaataaaggtaaatattaactgagtattttatcttatttaataataatattcctattatgttgtttatgtaacctgtttctaattaattcattaataaaataaaaaaactaaaaagagactaattcatttaaattaataaaattaaacagggactaataaaaaaaaactaattaaacatgaactaatttattaaaattaataaaaactaattaaacaggaactaattcattaaaattaaaaaaataaagagagactaattcatttaaattaataaaattaaagagagactaattaaaattaataaaactaattaaaaagggactaattcattaaaattaacaaaacttattaaacatgggcctaaaattaaaaaaataaaatgagacttattcatttaaatcatttttttatgaactcttgtaattttacattttacatgtgtttttaaaaattcacataatccttttaaatcttaaaaatctataaaggtctttcaattcttatgatataaatctattaaaatttaaattatcataaaaattttataaaaaaaaattaataaatcattacattcttatttaatctttaaaattttaaaaaaaattatgctaaaataatttttttaaatcttaattcaatacatcttctaaaatttttgtttttacccgataaaataataagtataacaaataattgtagcaaaaccagatgataaaagataaagaaaaaaggaaaaaaaaagggaaattttaaattacatatttactctaactttcatttttataaactattaatgaattttataaatataatttaatattgtgtgagccaaattgttattaggataacaatagagaggttgatctcaatagtagtcttttatcatcaaaatataccttgaggtgtaattataattactttattaataaattttataagtatttataagtatttttactatacatttttcacatgattttttcatattatatataatatattaattttgtactatatatttaacggttagaaacattaaatttattaataatttatattttttaaaattatctttaaaattaacatgtgctagtactttttgcaatcttcatacaagaaaatgaatttgtcttattaaattttaccatttattattaaataactaaaggtattttgataagaaaaaatcattagggatggaggtaatatttattaggaaaacattgtaaaacactaaaacccatcaggatatcgaccgaaggaacattttattactaaaccttaaaaagaaatattgcaataatcttcaaaattaagccattttaatttttattcttattgaatgataaaaaaaattaataattaaataacatttctcaaacactcttaaaccctagttgtttcctctgtctcaaaacaaaaaccctacaaacaaaacacttcctgcttcgcaaacgtttgaatctgaatc

SEQ ID NO: 59 - DNA, 484 bp, Arabidopsis thaliana
  source 1..484: mol_type = genomic DNA
aaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagcc

SEQ ID NO: 60 - DNA, 2066 bp, Glycine max
  source 1..2066: mol_type = genomic DNA
ggtatcagaaaccaaaggagacaacatctagatcagcaggtggaggatctaaacagctaggtgaatgagatccatcagaatatttgtacaagcctaaggatcttgtcttgccgtccctcatttaaagaagagtgttacatagatatctaacatactctctgtatcatttgtgtgatatatcatacttcaattatttttgcaaatcaattatgccattgttggtttagaattatatattttttgagtcactagaactagcgtcacaactgatgtgttaaattctctaatggttttgccaatgctgcaaggctaaaaatccgggttgaataatttaaggtaacttatgcaactaccactttagatatataataaaataaaattgcctaacttgtttttaaaaataaagatttttgtaacattaatgataaaaaaaaattaatttgtatgtcatgtttgatattcatgatttaacttagaattttgtaaaatctaatataaaattatgagatgagactcggaatcttccataatttatgacatcttacaaaactaagataaaatcaaactaataattttttttttccaatggacagctaaaatagaattggaacagtaaatatgtttgttaggctgtctgcacgcaaaagccaattcctgccgtaaatctctgcagtgaggcattctgttggacagtgttctgcggcttggagctaaaattttcgattccctctactttaaaagcattaccacgtcaatgcacgtgcatgtcaaggtcaaagagtaaattggttccaattgaagaatatcttgtgtccagctctctgttgatacagcaaaatgaatttcatctatcttccgtgagtaaacaagttcaatttgtgcttaaatttgttttatgaagagtgcaacctgaagcattgaaaacaatgaaaggcatcttgaaataatgaaatcatatatcttagatgtagacctccatccccgttaaaaagaaagtacactattttgggtgatggactcaatgactttaaagagagaaatttacgtacgctgacttttccttattttttttataagtaaacaacaacttaaataatagtttatataagtgaatatcaacttaaacaaatcgctcgcaactcactttccttctttaaagtcattgaatccttgccaattattttgtgagtatttgtaaatttaactttgaattattttgattttataaaattaattttagtaaaaagtgagttgaaattgaaatcatttatgtttatatttttttctcaatttcgtattaaagtttaagataaaacttttaacagagtaagagttatatttattttcatttttattgaaatatatgtttttaggagtaattttaattttggttaaaaatcaacaaacaactgttttttttttcttttgcaaaatctcattacacacggtgtcgtagaaccaaacatataattactaatacggatattactcattttagaattatgctgataattaatcgattaattataactttgctctcatttatatttttttcatttctctttatattgccctcttcattttacatcaaagtatccaaaaagtgtgactttgattaaattatatataccaaccttttttttataaaaaaaacctttcaaattgaaagactacaaatcagtgtggatttttacgtgacagataatattataatttcggtaaaattataatttggtcatttacttttttttatttagtcatctgttttatttattaattttgataacatatttcttatttttatttcaatttgatcttatttgtcacaaatttatttaacacaaaatacacacggacagataaagcaaatgaaaatgcaataaaagaaaatagcgggatcgctaaccgtttgctttaacgagcatcatcttattctcgtttcccgctctatatatattgattccctcacttcttcagacctcatttataattcaaaaacctctctctctctctctctctctctctcaattcttcaggcaccttcaaaatttctctctagagtcacg

SEQ ID NO: 61 - DNA, 1779 bp, Glycine max
  source 1..1779: mol_type = genomic DNA
ctcgaacatgttaaggaataagttactgtttgatgtgtacagagtttgttgtgtgtatctagccaagctttcttgatgctgttttgaacacgcgagttctccacagtatatacaagggtcttaagcataaataaatgaagaaatatggaacttctcctcattgatgtaatatatgttgtgaaaataaaacagttgtattatgaaccacagaatgatgtatgatgttttctcaacttgcattctaattttgatgtgttgctctaacacaatcacaatttatactctcaaaatttaaatctacgttcccacaattgtcaactatctttgcaggagtactgttaaagtctcaattacacactagggtgtgggatagaatttcccattttttttactagtaaaccctctttcttgaagttggactacacctgacaatttatttatttttcaaatctggaacatagtagacatcactgaatcactgactagctgaattatgtcattgatttgaagtcgagtaaaaatggtagagaaccacaaacattgatttaatttatgtcaaacattgatttgtcaaacaagctttgtttggggactaccttcctctgcctctccatcaaccgcaaacagaacctttacctttatctatatcggcaccttcaaagcttgttcatctcttgtatcttttgacaattcattctatactcattctacttttgtagctggtctattgtcatccaagttgttggacttctcagttgagcaaacatggtcaaatcttttctatactaacaactgatgtcatctttacacaataaatcttcatcttgtttacaattgtaagtgtccaagcaaagcactcatccactttctctccttctctaatctgaagaatttcaaactcccttttcaatattcgtaattgagatctcctcactctgatattagggtaagatatttttgtttcattgagtctcaaacgctattgaaactttccctatcaagaatagtctccacaatgtttccgtctctggcccaaaacagaacgtttttcatcttttattattctattgccttcgtttgcaatatttataaatgtttgaagaagttaggattatataataaatataaaagttacattagttcctattaaaattaaagaatttttttgaactaaattattatatctctgtaattgtttaccatgtatgcatttctcagaaaataattctgtttttttttttctttttaaacatgtattgcttttttagagtgtactttatatggatggagggagggaaggaaatgtctgatttttcatgttgatgtcaaatctaaatataaatacctacgcaaatttgggcaagtatgtataatgggcaaatgcatcacttaattattagaaattaggactctttagacaatggtcaatgttgtgttatagaaatgagcaatggggtatggtggtcattcatccttttatttagctgttcttcatgactaattatatatctcacttttgtggttctgattgcctaattgacaatgcaatctcaaaatagcccgggagtatcaagcattaacaaagctcagaaagaaggagagaaaaaagcgggtcaaaacaacaatttcaaccatcaacaaataaagggatgaatgagaacctcacattaagcggacccatattttttataaaacgcaatcctctcttccttgaacacagtagtacattttcctctgaaccaaaccctcatccaaattaaccagcaagcggtattcaca

SEQ ID NO: 62 - protein, 1264 aa, synthetic construct
MLFQDFTHLYPLSKTMRFELKPIGKTLEHIHAKNFLSQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHRNRKNMYSDEDKHTAITYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGALYDELAKIPTLYNKVRDYLSQKPFSTEKYKLNFGNPTLLNGWDLNKEKDNFGIILQKDGCYYLALLDKAHKKVFDNAPNTGKNVYQKMIYKLLPGPNKMLPKVFFAKSNLDYYNPSAELLDKYAQGTHKKGNNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSKDNLANPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDEADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWKICSHGDKRYVYDKTANQNKGATKGINVNDELKSLFARHHINDKQPNLVMDICQNNDKEFHKSLIYLLKTLLALRYSNASSDEDFILSPVANDEGMFFNSALADDTQPQNADANGAYHIALKGLWVLEQIKNSDDLNKVKLAIDNQTWLNFAQNR

SEQ ID NO: 63 - protein, 1319 aa, synthetic construct
MLFQDFTHLYPLSKTMRFELKPIGKTLEHIHAKNFLSQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHRNRKNMYSDEDKHTAITYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGALYDELAKIPTLYNKVRDYLSQKPFSTEKYKLNFGNPTLLNGWDLNKEKDNFGIILQKDGCYYLALLDKAHKKVFDNAPNTGKNVYQKMIYKLLPGPNKMLPKVFFAKSNLDYYNPSAELLDKYAQGTHKKGNNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSKDNLANPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDEADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWKICSHGDKRYVYDKTANQNKGATKGINVNDELKSLFARHHINDKQPNLVMDICQNNDKEFHKSLIYLLKTLLALRYSNASSDEDFILSPVANDEGMFFNSALADDTQPQNADANGAYHIALKGLWVLEQIKNSDDLNKVKLAIDNQTWLNFAQNRPKKKRKVKRPAATKKAGQAKKKKGGSGGSGGSGGSGGSGGSGGSGGSLEHHHHHH